(19) United States
(12) Patent Application Publication
(10) Pub. No.: US 2005/0015821 A1
(43) Pub. Date: Jan. 20, 2005

(54) Sensin polypeptides, encoding nucleic acids, mutations, and methods of their identification and use

(75) Inventors: Richard J. Glynne, La Jolla, CA (US); Nancy Ai-Hua Hong, San Diego, CA (US); Keats A. Nelms, Turner (AU); Hua Wu, San Diego, CA (US)

Correspondence Address: FOLEY & LARDNER, P.O. BOX 80278, SAN DIEGO, CA, 92138-0278, US

(73) Assignee: Phenomix Corporation

(21) Appl. No.: 10/783,989

(22) Filed: Feb. 20, 2004

Related U.S. Application Data
(60) Provisional application No. 60/448,964, filed on Feb. 21, 2003.

(51) Int. Cl. A01K 67/027

(52) U.S. Cl. 800/18

(57) ABSTRACT

The present invention relates to the discovery, identification, and characterization of novel genes encoding proteins that share sequence similarity with animal zinc finger proteins. The invention encompasses the described polynucleotides, the encoded proteins, fusion proteins, polypeptides and peptides, genetically engineered animals that either under- or over-express the disclosed sequences, antibodies to the encoded proteins and peptides, host cell expression systems, antagonists and agonists of the proteins, and other compounds that modulate the expression or activity of the proteins encoded by the disclosed sequences that can be used for diagnosis, drug screening, clinical trial monitoring and the treatment of diseases and disorders.

### FIELD OF THE INVENTION

The present invention relates to the discovery, identification, and characterization of novel genes and proteins related to neuromuscular function.

### BACKGROUND OF THE INVENTION

The brain controls the movements of skeletal (voluntary) muscles via specialized nerves. The combination of the nervous system and muscles, working together to permit movement, is known as the neuromuscular system. If a person wants to move part of their body, a message is sent to upper motor neurons on the surface of the brain. These neurons have long axons that descend into and through the brain, and into the spinal cord. At the spinal cord, lower motor neurons ultimately branch into the muscle tissue itself.

Muscles may be serviced by anywhere between 50 and 200 (or more) lower motor neurons. Each lower motor neuron is subdivided into many tiny branches. The tip of each branch is called a presynaptic terminal. Signal from the brain travel down the nerves and prompt the release of the transmitter acetylcholine from the presynaptic terminals. This transmitter is bound by special receptors in the muscle tissue. If enough receptors are stimulated by acetylcholine, the result is muscular contraction.

There are many separate diseases that are classified as neuromuscular disorders. The symptoms of neuromuscular disease vary according to the condition and may be mild, moderate or life threatening. Some of these symptoms may include muscular weakness, muscle wastage, muscular cramps, muscle spasticity, loss of coordination, muscle pain, skin rash, breathing difficulties, swallowing difficulties, etc. The diseases affecting the neuromuscular system may be classified into the following broad groups:

Dystrophies—muscular dystrophy (muscle wastage) is usually caused by a genetic mutation that prevents the maintenance and repair of muscle tissue. Some of the different types include Becker muscular dystrophy, congenital muscular dystrophy, Duchenne muscular dystrophy and facioscapulohumeral muscular dystrophy.

Atrophies—the nerve system of the spinal cord is affected and the upper and lower motor neurons waste away. Some of the different types of motor neuron disease include infantile progressive spinal muscular atrophy (SMA1), intermediate spinal muscular atrophy (SMA2), juvenile spinal muscular atrophy (SMA3) and adult spinal muscular atrophy.

Neuropathies—the peripheral nervous system (nerves other than those within the spinal cord) are affected. Some of the different diseases of the peripheral nerve include Charcot-Marie-Tooth disease and Friedrich's ataxia.

Inflammatory myopathies—muscle diseases characterized by inflammation. Some of the different types of inflammatory myopathy include dermatomyositis and polymyositis.

Diseases of the neuromuscular junction—the acetylcholine receptors on the muscle tissue are targeted by inflammation. Some of the different diseases of the neuromuscular junction include Guillain-Barre syndrome, myasthenia gravis and polyneuropathy.

Acquired nerve diseases—polyneuropathies can be triggered by certain drugs (chemotherapeutic agents and medications for lowering blood cholesterol), dietary deficiencies (vitamin B12) and hormonal disorders (diabetes and hypothyroidism).

Compression neuropathies—nerves may become compressed by other structures and tissues (such as bones), leading to impairment of function and symptoms such as pain, numbness and tingling. Some of the different types of compression neuropathies include carpal tunnel syndrome and sciatica.

Metabolic diseases—the muscles cannot use certain crucial nutrients and this affects their functioning. Some of the metabolic diseases of muscle include phosphofructokinase deficiency, acid maltase deficiency and carnitine deficiency.

The causes of neuromuscular disease vary according to the condition. Some of the causes and risk factors may include nerve damage (head injury, stroke), viral infection, hormonal or metabolic disorders, and dietary deficiencies. In addition, the advent of modern genomics has lead to an understanding of various genetic disorders that cause neuromuscular disease. See, e.g., “Neuromuscular disorders: gene location,” Neuromuscul. Disord. 12: 790-800 (2002); “Congenital myasthenic syndromes: gene mutations,” Neuromuscul. Disord. 12: 807-11 (2002); “Mutations in GDAP1: autosomal recessive CMT with demyelination and axonopathy,” Neurology 59: 1865-72 (2002); “A novel form of autosomal recessive pure hereditary spastic paraplegia maps to chromosome 13q14,” Neurology 59: 1905-9 (2002); “SPG3A: An additional family carrying a new atlastin mutation,” Neurology 59: 2002-5 (2002).

Neuromuscular disorders are diagnosed using a range of tests, including blood tests, muscle biopsies and genetic testing. Treatment varies widely, according to the individual disorder, and some conditions are more easily treated than others. For example, compression neuropathies such as carpal tunnel syndrome can be eased with cortisone injections, fluid tablets (to reduce the swelling) and surgery. For other forms of neuromuscular disease, such as muscular dystrophies and motor neuron diseases, there is currently no cure or successful treatment.

### SUMMARY OF THE INVENTION

The present invention describes the discovery, identification, and characterization of polynucleotides that encode novel proteins, and the corresponding amino acid sequences of these proteins. The novel proteins described for the first time herein share sequence similarity with animal zinc finger proteins, and particularly RING finger proteins. The novel proteins have been designated “Sensin.” The analysis of mutations in Sensin and Sensin-like proteins described herein confirm that deficits in Sensin function are causative of adult-onset motor dysfunction in animals, including mammals.

The understanding of the key role played by Sensin and Sensin-like proteins in normal neuromuscular function indicates that Sensin itself provides a novel diagnostic and therapeutic target. In addition, the demonstration of homology with zinc finger and RING finger proteins indicates a likely means by which proteins and nucleic acids interact with Sensin and Sensin-like proteins in biological systems. These secondary proteins and nucleic acids together with Sensin provide a novel pathway by which normal neuromuscular function is maintained. Thus, any protein in this sensin-mediated pathway also provides a diagnostic and therapeutic target Genetically engineered animals that either under- or over-express the disclosed sequences, antibodies to the encoded proteins and peptides, host cell expression systems, antagonists and agonists of the proteins, and other compounds that modulate the expression or activity of the proteins encoded by the disclosed sequences that can be used for diagnosis, drug screening, clinical trial monitoring and the treatment of diseases and disorders are described herein.

The term “zinc finger” as used herein refers to a motif well known to those of skill in the art associated with DNA-binding and ubiquitination proteins. A classical zinc finger comprises about 12 amino acids containing two cysteine and two histidine residues (referred to as a “C2H2 zinc finger”) that directly coordinate a zinc atom. However, other patterns of amino acids are found within this family, including “CCCC” and “CCHC”. Zinc fingers typically mediate binding to either DNA or protein.

Zinc finger domain polypeptide can function in DNA binding or protein ubiquitination. These activities regulate gene transcription or protein degradation, respectively, and are therefore involved in numerous biological processes, including maintenance of neuronal viability. See, e.g., Imai (2000) J. Biol. Chem. 275(46):35661-35664.

The term “RING finger” as used herein refers to a specialized type of zinc finger of 40 to 60 residues that binds two atoms of zinc. There are at least two different variants, the C3HC4-type and a C3H2C3-type, which exhibit different cysteine/histidine patterns within the motif. RING finger proteins can contain intrinsic ubiquitin ligase activity within the RING finger motif. See, e.g., Joazeiro (1999) Science 286(5438):309-312.

In a first aspect, the present invention relates to nucleic acids encoding wild-type Sensin polypeptides, and polypeptides encoded by such nucleic acids. In a related aspect, the present invention relates to nucleic acids encoding mutated Sensin polypeptides, and polypeptides encoded by such nucleic acids. The nucleic acids of the present invention include genomic DNA sequences, RNA sequences, cDNA sequences, introns, exons, etc.

In preferred embodiments, the mutated Sensin nucleic acids encode polypeptides comprising one or more mutations in a zinc finger motif and/or one or more mutations in an intronic sequence that alters the length of an expressed protein relative to wild-type. In particularly preferred embodiments, the mutated Sensin nucleic acids produce an altered motor-related phenotype in an animal expressing the mutated Sensin nucleic acid.

The term “Sensin nucleic acid” as used herein refers to a nucleic acid encoding a Sensin polypeptide, including, but not limited to, a genomic nucleic acid, hnRNA, mature mRNA, cDNA; amplification products thereof; and the Watson-Crick complements thereof. Preferred Sensin nucleic acids are the mouse and human wild-type Sensin nucleic acids having the sequences of SEQ ID NO: 1 and 2. Preferably, Sensin nucleic acids comprise at least 15 consecutive nucleotides, preferably at least 20 consecutive nucleotides, more preferably at least 40 consecutive nucleotides, and most preferably at least 60 consecutive nucleotides of the sequences of SEQ ID NO: 1 and 2. A Sensin nucleic acid, protein, or mutated version thereof, of the present invention may be obtained from numerous species, including a humans and non-human animal species such as primate, caprine, bovine, ovine, porcine, and murine species. Most preferably, a Sensin nucleic acid, protein, or mutated version thereof, of the present invention is obtained from mouse.

The term “altered motor-related phenotype” as used herein refers to one or more detectable characteristics of movement in an animal that differ from normal movement that is typically characteristic in that animal's species.

The terms “expression” and “expressing” as used herein in reference to nucleic acid sequences refers to the translation of a nucleic acid sequence by cellular machinery to provide a polypeptide.

The term “Sensin genomic sequence” as used herein refers to a nucleic acid sequence encoding all or a portion of a Sensin polypeptide in which any intronic and extronic sequences present in the nucleic acid sequence are in the same linear organization as in the Sensin gene from which the Sensin genomic sequence arises. The Sensin gene organization from mouse is described in detail hereinafter.

The term “Sensin polypeptide” as used herein refers to a polypeptide of between 1500 to 1800 amino acids comprising at least one, and more preferably at least two N-terminal lysine-rich domains having greater than 25% lysine-containing residues in a total length of from 9-16 amino acids; and a C-terminal zinc finger domain that bears similarity to the RING finger subfamily (containing at least eight cysteine/histidine residues in a conserved spacing as well as other conserved residues within a 50-amino acid domain). Preferably, a Sensin Polypeptide is at least about 90%, preferably at least about 95%, and most preferably at least about 98% identical to an equal length segment of a wild-type Sensin polypeptide. The term “about” in this context refers to +/−1% of a given measurement. The terms “wild-type mouse Sensin polypeptide” and “wild-type human Sensin polypeptide” as used herein refers to the mouse and human wild-type Sensin polypeptides having the sequences of SEQ ID NO: 7 and 8.

The present invention also relates to fragments of such Sensin nucleic acids and polypeptides. Preferably, a Sensin nucleic acid fragment comprises at least 15 consecutive nucleotides, preferably at least 20 consecutive nucleotides, more preferably at least 40 consecutive nucleotides, and most preferably at least 60 consecutive nucleotides of a Sensin nucleic acid; and a Sensin polypeptide fragment comprises at least 5 consecutive amino acid residues, preferably at least 7 consecutive amino acid residues, more preferably at least 10 consecutive amino acid residues, even more preferably at least 15 consecutive amino acid residues, and most preferably at least 20 consecutive amino acid residues of a Sensin polypeptide.

The term “mutated Sensin nucleic acid” as used herein refers to a nucleic acid encoding a mutated Sensin polypeptide, including, but not limited to, a mutated Sensin genomic sequence, a mutated hnRNA, a mutated mature mRNA, a mutated cDNA; amplification products thereof; and the Watson-Crick complements thereof.

The term “mutated Sensin polypeptide” refers to a polypeptide that is at least about 90%, preferably at least about 95%, and most preferably at least about 98% identical to an equal length segment of a wild-type Sensin polypeptide obtained from the same species. The term “about” in this context refers to +/−1% of a given measurement. Protein identity is determined by aligning two sequences using BLAST (Altschul, et al., “Gapped BLAST and PSI-BLAST: a new generation of protein database search programs.” Nucleic Acids Res. 25:3389-3402(1997)) with default parameters, and determining the number of identical amino acids relative to the total length of the mutated Sensin polypeptide. Preferably, a mutated Sensin polypeptide produces an altered motor-related phenotype when expressed in an animal.

The present invention also relates to fragments of such mutated Sensin nucleic acids and polypeptides. Preferably, a mutated Sensin nucleic acid fragment comprises at least 15 consecutive nucleotides, preferably at least 20 consecutive nucleotides, more preferably at least 40 consecutive nucleotides, and most preferably at least 60 consecutive nucleotides of a mutated Sensin genomic sequence, hnRNA, mature mRNA, cDNA, or the complement thereof, which includes the site of the mutation; and a mutated Sensin polypeptide fragment comprises at least 5 consecutive amino acid residues, preferably at least 7 consecutive amino acid residues, more preferably at least 10 consecutive amino acid residues, even more preferably at least 15 consecutive amino acid residues, and most preferably at least 20 consecutive amino acid residues of a mutated Sensin polypeptide, which includes the site of the mutation.

While mutations in the mutated Sensin nucleic acids and polypeptides of the present invention may include deletions, substitutions, inversions, insertions, etc., it is preferred that the nucleic acids of the present invention encode polypeptides that are reduced in length to their corresponding wild-type counterparts; most preferably, the mutation is caused by an alteration in an intronic sequence, resulting in altered RNA size (e.g., a mis-splicing event) relative to a wild-type RNA.

In another aspect, the present invention relates to one or more transgenic animals that are comprise one or more non-wild type Sensin genes. The skilled artisan will understand that a gene is present in a diploid genome as two alleles. In various embodiments, such transgenic animals may comprise a mutated Sensin transgene in addition to or in place of one or both wild type alleles; may be “knockout” animals (which can be conditional) that do not express a functional Sensin or have reduced levels of Sensin expression. Animals may also comprise endogenous Sensin nucleic acids and/or mutated Sensin nucleic acids. The term “endogenous” as used herein in reference to cells refers to a nucleic acid sequence that is present in cells without the use of recombinant DNA techniques. Various animal cells will naturally contain Sensin nucleic acid sequences and/or mutated Sensin nucleic acid sequences.

Endogenous Sensin nucleic acid sequences may be mutated, e.g., using mutagens such as: radiation (gamma, beta, alpha, UV, etc.); base analogues such as bromouracil and aminopurine; chemicals such as nitrous acid, nitrosoguanidine, ethylnitrosourea, and ethylmethanesulfonate; intercalating agents such as acridine orange and ethidium bromide, to provide endogenous mutated Sensin family nucleic acid sequences. In exemplary embodiments, the present invention describes a mouse line that carries a mutated version of the endogenous Sensin gene resulting in an altered motor-related phenotype. Exemplary methods for providing endogenous mutated Sensin nucleic acid sequences are described hereinafter.

The animals of the present invention may be used for a variety of purposes, including the production of proteins encoded by expression constructs, screening methods to identify modulators of Sensin polypeptides, testing the effects of such modulators on altered motor-related phenotypes, identifying genetic modulators of altered motor-related phenotypes, sensitized screening methods, etc.

In another aspect, the present invention relates to agonists and antagonists of Sensin protein, including small molecules, large molecules, mutant Sensin proteins, or portions thereof that alter the function of Sensin proteins or compete with native proteins, peptides, and antibodies, as well as nucleotide sequences that can be used to inhibit the expression of Sensin proteins (e.g., antisense, ribozyme, decoy oligos, and RNAi molecules, and gene or regulatory sequence replacement constructs) or to enhance the expression of Sensin proteins (e.g., expression constructs that place the described sequences under the control of a strong promoter system). The skilled artisan will understand that such agonists and antagonists may be active at wild-type sensin protein, mutated sensin protein, or both. Likewise, the skilled artisan will understand that function or expression of Sensin may also be affected by agonists and antagonists upstream or downstream in the Sensin-mediated motor pathway.

Further, the present invention also relates to processes for identifying compounds that modulate, i.e., act as agonists or antagonists, of Sensin expression and/or Sensin activity that utilize purified preparations of the Sensin gene product (either mutated, wild-type, or both), or cells expressing the same. Such compounds can be used as therapeutic agents for the treatment of any of a wide variety of symptoms associated with biological disorders or imbalances.

In another aspect, the present invention relates to proteins that interact with the Sensin protein (either mutated, wild-type, or both) and modulate or are modulated by Sensin protein activity. Such proteins can be identified by methods which may include available in vivo or in vitro based screening systems to detect the interactions between two proteins. For instance, commonly available genetic systems are capable of rapidly detecting which proteins interact with a known protein, determining which domains of the proteins interact and identifying agents which modulate the interaction between two proteins. One such system is the yeast two-hybrid system wherein two proteins are expressed in yeast: one protein of interest fused to a DNA-binding domain and the other protein of interest fused to a transcriptional activation domain (Fields (1989) Nature 340(6230):245-246).

In a related aspect, the invention encompasses proteins that act as Sensin substrates in an enzymatic reaction. In a preferred embodiment, these substrates are identified in an enzymatic reaction measured by an ubiquitination assay. This assay is performed by (i) providing a ubiquitin-conjugating system that includes the candidate substrate protein, the Sensin protein, and ubiquitin under conditions which promote the ubiquitination of the substrate protein, and (ii) measuring the level of ubiquitination of the substrate protein brought about by the system. The level of ubiquitination of the substrate protein can be measured by determining the actual concentration of protein:ubiquitin conjugates formed; or inferred by detecting some other quality of the substrate protein affected by ubiquitination, including the proteolytic degradation of the protein. In certain embodiments, the present assay comprises an in vivo ubiquitin-conjugating system, such as a cell able to conduct the substrate protein through at least a portion of a ubiquitin-mediated proteolytic pathway. In other embodiments, the present assay comprises an in vitro ubiquitin-conjugating system comprising a reconstituted protein mixture in which at least the ability to transfer ubiquitin to the substrate protein is constituted.

The term “substrate” as used herein refers to a molecule that can directly or indirectly associate with a protein of interest to mediate activity in one or more biologically relevant pathways.

The invention also encompasses agonists and antagonists of said Sensin protein substrates, including small molecules, large molecules, mutant substrate proteins, or portions thereof that compete With native proteins, peptides, and antibodies, as well as nucleotide sequences that can be used to inhibit the expression of substrate proteins (e.g., antisense, ribozyme, and RNAi molecules, and gene or regulatory sequence replacement constructs) or to enhance the expression of substrate proteins (e.g., expression constructs that place the described sequences under the control of a strong promoter system).

Further, the present invention also relates to processes for identifying compounds that modulate, i.e., act as agonists or antagonists, of Sensin substrate expression and/or Sensin substrate activity that utilize purified preparations of the Sensin substrate product, or cells expressing the same. Such compounds can be used as therapeutic agents for the treatment of any of a wide variety of symptoms associated with biological disorders or imbalances.

In another aspect, the present invention relates to recombinant DNA vectors comprising a Sensin nucleic acid, and/or a mutated Sensin nucleic acid. Preferably, such a vector is an expression construct in which a Sensin nucleic acid, and/or a mutated Sensin nucleic acid, is operably inserted downstream in the direction of transcription of a transcriptional regulatory region functional in a cellular expression host. Vectors, plasmids or viruses, may be used to prepare such recombinant DNA vectors. In preferred embodiments, recombinant DNA vectors of the present invention will usually have a gene for positive selection, e.g. antibiotic, enzyme that produces a detectable substrate, etc., and may have other features known to those of skill in the art for integration or permanent maintenance, i.e. replication.

The term “recombinant DNA vector” as used herein refers to a circular or linear DNA molecule that contains an inserted piece of DNA, and that is capable of replication in certain host cells. A recombinant DNA vector may be replicated without integration into the host cell genome, or may be integrated into the genome. Such vectors may be capable of replicating in prokaryotic cells, in eukaryotic cells, or both. Reco promoter which is capable of initiating the 5′ synthesis of RNA from cDNA is selected from the group consisting of the MoMLV promoter, metallothionein promoter, glucocorticoid promoter, SV 40 promoter, and the CMV promoter recombinant DNA vectors are well known to those of skill in the art. See, e.g., U.S. Pat. Nos. 6,391,585; 6,342,372; 6,326,195; 6,210,939; 6,057,152; 5,665,578; 5,646,009; and 5,604,118.

Preferred recombinant DNA vectors of the present invention comprise a sequence encoding the mouse wild-type Sensin polypeptide having the sequence of SEQ ID NO: 7, most preferably encoded by the nucleic acid of SEQ ID NO: 1. Other preferred recombinant DNA vectors of the present invention comprise a nucleic acid sequence encoding a mutated Sensin polypeptide having the sequence of SEQ ID NO: 5. Other preferred nucleic acids encode polypeptides having the sequences of SEQ ID NO: 6, 8, 9, 10, or 11

The term “expression construct” as used herein refers to a recombinant DNA vector comprising regulatory regions providing for transcription and translation of an inserted piece of DNA when propagated in suitable host cells. In addition to an origin of replication and a selection gene, an expression construct typically contains a promoter, a ribosome binding site, and a transcription terminator. Suitable promoters include both eukaryotic and prokaryotic promoters such as, for example, b-galactosidase promoter, trpE promoter, lacZ promoter, T7 promoter, T3 promoter, SP6 promoter, the MoMLV promoter, metallothionein promoter, glucocorticoid promoter, SV 40 promoter, and the CMV promoter. Additional optional elements, such as enhancers, multiple cloning sites, etc., may also be present.

In another aspect, the recombinant DNA vectors of the present invention may be inserted into suitable eukaryotic or prokaryotic cells to provide “host cells” that comprise a Sensin nucleic acid or mutated Sensin nucleic acid, e.g., using transfection or transformation techniques well known to those of skill in the art. Eukaryotic host cells may be obtained or derived from numerous animal species, including a humans and non-human animal species such as primate, caprine, bovine, ovine, porcine, and murine species.

Preferably, such host cells are cells, other than an intact animal, comprising one or more introduced Sensin nucleic acids; and cells, other than an intact animal, comprising one or more introduced mutated Sensin nucleic acids. In particularly preferred embodiments, such cells express the introduced nucleic acid.

The term “introduced” as used herein in reference to cells refers to a nucleic acid sequence inserted into one or more cells by recombinant DNA techniques. An introduced Sensin nucleic acid may be, for example, a Sensin nucleic acid obtained from one species and introduced into a cell of a different species, such as a nucleic acid encoding a human Sensin protein inserted into a mouse cell or vice versa. Alternatively, an introduced Sensin nucleic acid encoding a human Sensin protein may be inserted into a human cell, or a nucleic acid encoding a mouse Sensin protein may be inserted into a mouse cell, using recombinant DNA techniques. Preferably, an introduced Sensin nucleic acid is in a recombinant DNA vector, and the vector is inserted into a cell.

Host cells prepared according to the present invention may be used for a variety of purposes, including ex vivo gene transfer methods, the production of proteins encoded by expression constructs, screening methods to identify modulators of Sensin polypeptides, etc.

Suitable cells for use in such methods include host cells comprising one or more introduced wild-type or mutated Sensin nucleic acids, as described herein, and/or cells comprising one or more endogenous wild-type or mutated Sensin nucleic acids. Cells may be contacted in an in vitro environment (e.g., in a cell culture), or in an in vivo environment (e.g., cells in a live fetus or animal). Typically, the presence, amount, or function of endogenous wild-type or mutated Sensin nucleic acids in or from cells treated with the composition(s) will be compared to one or more control cells not so treated. Preferably, both cells to be tested and control cells express the wild-type or mutated Sensin nucleic acid(s) constitutively.

In these cell-based screening methods, the skilled artisan will understand that a test composition may affect the characteristics of the polypeptide at a number of levels, including transcription of the Sensin nucleic acid(s), translation of Sensin nucleic acid(s), or the activity of Sensin polypeptide produced. Additionally, such screening methods may rely on directly measuring the presence, amount, or activity of the wild-type or mutated Sensin DNA, RNA, or protein; or may rely on indirect measurements.

In particularly preferred embodiments, suitable test compositions include those having a direct effect on the properties of a mutated Sensin polypeptide, such as compounds and libraries are derived from ubiquitination-modulating compounds known in the art (see, e.g., Berleth, J. Biol. Chem. 267(23):16403-16411(1992); and Swinney, J. Biol. Chem. 277(26):23573-23581 (2002)); double-stranded RNA designed to provide gene silencing of the mutated Sensin nucleic acid(s) by RNA interference (“RNAi”) (see, e.g., Paddison et al., Proc. Nat'l Acad. Sci. USA 99: 1443-8 (2002); and Hutvagner and Zamore, Curr. Opin. Genet. Dev. 12: 225-32 (2002)); antisense nucleic acids designed to inhibit expression of the mutated Sensin nucleic acid(s) (see, Bavisotto, J. Exp. Med. 174: 1097-1101 (1991)); gene therapy constructs designed to disrupt a Sensin gene (“knockout” constructs); gene therapy constructs designed to overexpress Sensin nucleic acid(s), thereby compensating for the presence of mutated Sensin polypeptides; gene therapy constructs designed to overexpress Sensin nucleic acid(s), thereby compensating for phenotypes related to Sensin polypeptide function; decoy oligonucleotides designed to bind to a mutated Sensin polypeptide but not to a wild-type Sensin polypeptide (see, Mann and Dzau, J. Clin. Invest. 106: 1071-75 (2000)); or a combination of any of these compositions. This list is not mean to be limiting, and other appropriate test compositions will be apparent to those of skill in the art.

Numerous species, including a humans and non-human animal species such as primate, caprine, bovine, ovine, porcine, and murine species, may be used in such screening methods. In preferred embodiments where screening is performed in vivo, the animals used are mice. In yet other preferred embodiments, the effect of such compositions on motor-function is determined.

Preferably, the cells and/or animals used in the screening methods of the present invention comprise a nucleic acid sequence encoding a Sensin polypeptide, most preferably having the sequence of SEQ ID NO: 7 or SEQ ID NO: 5. Preferred nucleic acids encoding such polypeptides include those having the sequences of SEQ ID NO: 1.

In another aspect, the present invention relates to methods of identifying mutations in a Sensin gene. Such mutations may produce a dominant or recessive motor-related phenotype in an animal. These methods comprise identifying one or more animals exhibiting such a dominant or recessive altered phenotype, and determining whether a mutated Sensin gene is present in said animal.

A phenotype is said to be “recessive” if the relevant characteristics detectable in a homozygotic animal are not apparent in an animal that is heterozygotic for the mutation of interest. A phenotype is said to be “dominant” if the relevant characteristics detectable in a heterozygotic animal.

Numerous animal species, including a humans and non-human animal species such as primate, caprine, bovine, ovine, porcine, and murine species, may be used for identifying mutations in a Sensin gene that produce a recessive altered motor-related phenotype according to the present invention. In preferred embodiments, the animals used are mice.

Once one or more animals exhibiting a recessive altered motor-related phenotype are identified, Sensin gene sequences can be determined using sequencing methods that are well known to those of skill in the art. In preferred embodiments, mutations are identified by comparison to the wild-type Sensin nucleic acid having the sequence of SEQ ID NO: 1. Alternatively, Sensin mRNA, cDNA, and/or polypeptides may be sequenced. In preferred embodiments, Sensin mutations can be identified by comparison to proteins having the sequence of SEQ ID NO: 7.

In another aspect, the present invention relates to methods of identifying subjects in need of compositions modulating one or more characteristics of mutated Sensin polypeptides, and methods for administering such compositions to a subject. The methods comprise contacting one or more cells of the subject expressing mutated Sensin polypeptides with one or more compositions that affect one or more characteristics of the mutated polypeptide.

The term “subject” as used herein refers to a human or a non-human animal. Thus, the methods and compositions described herein can be used for both medical and veterinary purposes.

A subject may be identified as being in need of administration of one or more compositions that affect one or more characteristics of the mutated Sensin polypeptides by numerous methods well known to those of skill in the art. For example, the presence, amount, or activity of the mutated Sensin genomic DNA, immature or mature messenger RNA, or expressed protein may be determined.

Alternatively, or together with determining mutated Sensin DNA, RNA, or protein, indirect measurements, such as identification of an altered motor-related phenotype in a subject may also be used to identify suitable subjects. Preferably, administration of such composition(s) ameliorates one or more of these phenotypes, i.e., returns an aberrant phenotype to a normal or increasingly normal phenotype.

Such methods may advantageously be used to diagnose patients suffering from or at risk for motor-related diseases, or to identify homozygous carrier states, and for treatment of patients with compounds that have an effect on motor function. The skilled artisan will further understand that such methods may also be used to monitor the course of treatment of a subject with compositions that affect one or more characteristics of the mutated Sensin polypeptides. For example, limb muscle strength in such a subject may be monitored for possible improvement by such treatment.

In another aspect, the present invention relates to kits for determining the presence or amount of a mutated Sensin DNA, RNA, or protein in a sample. Such kits preferably comprise one or more assay components for detecting the mutated Sensin DNA, RNA, or protein, and may optionally include one or more of: instructions for performing the detection; reagents, such as buffers, for use in performing the detection; pipettes for liquid transfers; etc.

A suitable assay component may comprise an antibody, or a fragment or variant thereof, may be provided that is capable of specifically binding mutated Sensin. A method of raising the antibody preferably comprises isolating the antibody from an animal or isolating an antibody-producing cell from an animal, following administration of mutated Sensin protein, or an antigenic fragment thereof, to the animal. Additional methods for obtaining antibody-like molecules, such as single chain variable region fragments obtained from phage display, are also well known to those of skill in the art. An antibody of the invention may be useful in detecting or measuring the presence of mutated Sensin protein in an individual, by contacting the antibody with a biological sample from a subject.

The term “specifically binds” as used herein with regard to antibodies does not indicate that there is no binding of the antibody to non-target protein(s). Rather, an antibody is defined as being “specific for”, as “specifically reacting with”, or as “specifically binding to”, target protein(s) if the antibody exhibits a binding affinity for target protein that is at least about twice the affinity exhibited for a non-target protein. Certain preferred antibodies exhibit a binding affinity for the mutated Sensin protein of interest that is at least about twice the affinity exhibited for binding to non-Sensin proteins. Other preferred antibodies exhibit a binding affinity for the mutated Sensin protein of interest that is at least about twice the affinity exhibited for a Sensin protein lacking the mutation.

A suitable assay component may also comprise a nucleic acid that hybridizes under stringent conditions to a mutated Sensin nucleic acid of interest, but that does not hybridize under such conditions to a Sensin nucleic acid lacking the mutation. Such “probe” nucleic acids are at least 15 nucleotides in length, more preferably at least 20 nucleotides in length, and even more preferably 30 nucleotides in length or more, and contain nucleotide(s) corresponding to the mutation of interest.

Preferred nucleic acids may be substantially complementary to a wild-type or mutated Sensin nucleic acid of interest. By “substantially complementary” is meant that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target sequence, positioned 3′ or 5′ to a contiguous sequence of bases that hybridize under stringent hybridization conditions to a target sequence.

The term “stringent hybridization conditions” refers to 42° C. in 50% formamide, 5×SSPE, 0.3% SDS, and a repetitive sequence blocking nucleic acid, such as cot-i or salmon sperm DNA (e.g., 200 n/ml sheared and denatured salmon sperm DNA).

A suitable assay component may also comprise a nucleic acid suitable for use in a primer extension assay for detection of a wild-type or mutated Sensin nucleic acid of interest. As used herein, “primer extension” refers to the enzymatic extension of the three-prime (3′) hydroxy group of an extension primer, which is an oligonucleotide that is paired in a duplex to a template nucleic acid. For an example of primer extension as applied to the detection of polymorphisms, see Fahy et al., Multiplex fluorescence-based primer extension method for quantitative mutation analysis of mitochondrial DNA and its diagnostic application for Alzheimer's disease, Nucleic Acid Research 25:3102-3109, 1997. The extension reaction is catalyzed by a DNA polymerase. Extension of the 3′ end of the oligonucleotide generates an oligonucleotide having a length greater than the extension primer and having a sequence that is the reverse complement of the template nucleic acid. If one of the nucleotides in the added sequence is labeled, then the extended oligonucleotide becomes labeled. Extension primers must be of a length sufficient to provide specific binding to the target sequence of interest. The extension primer sequence has a 3′ terminus that pairs with a nucleotide base that is, in the sample nucleic acid to which the primer is hybridized, 5′ from the site of one or more bases in the sequence of interest that represent a mutation of interest.

By “DNA Polymerase” it is meant a DNA polymerase, or a fragment thereof, that is capable of catalyzing the addition of bases to a primer sequence in a sequence-specific fashion. A DNA polymerase can be an intact DNA polymerase, a mutant DNA polymerase, an active fragment from a DNA polymerase, such as the Klenow fragment of E. coli DNA polymerase, and a DNA polymerase from any species, including but not limited to thermophilic organisms.

The term “biological sample” as used herein refers to a sample obtained from a cell, tissue, or organism. Examples of biological samples include proteins and/or nucleic acids obtained from cells (e.g., mammalian cells, bacterial cells, cultured cells), particularly as a lysate, a biological fluid (such as blood, plasma, serum, urine, bile, saliva, tears, cerebrospinal fluid, aqueous or vitreous humor, or any bodily secretion), a transudate or exudate (e.g., fluid obtained from an abscess or other site of infection or inflammation), a fluid obtained from a joint (e.g., a normal joint or a joint affected by disease such as rheumatoid arthritis, osteoarthritis, gout or septic arthritis), or the like.

The summary of the invention described above is not limiting and other features and advantages of the invention will be apparent from the following detailed description of the preferred embodiments, as well as from the claims.

### BRIEF DESCRIPTION OF FIGURES

FIG. 1 contains a schematic of the mouse sensin gene locus. The genomic locus of mouse sensin gene is shown with coordinates based on the public Mouse Genome Sequencing Consortium Version 3 genomic assembly.

FIG. 2 illustrates the mutant transcript expressed from the mutated mouse sensin gene. The wild-type and mutant mouse Sensin transcripts in the region of the point mutation in mutant Sensin mice are shown.

FIG. 3 shows the expression pattern of the mouse sensin gene in the adult nervous system. Fluorescent in situ hybridization of adult mouse cerebellum (left panels) and spinal cord (right panels) with probes for negative control (top panels) and mouse sensin gene (bottom panels) is shown.

FIG. 4 contains sequencing data of the wildtype and mutant mouse sensin genes.

FIG. 5 shows the histopathology of mice homozygous for the mutant sensin gene as revealed by nissl staining of dorsal horn spinal cord neurons and glia. The mutant sample (right) has excessive vacuoles in the neuropil (arrows) and more small cell body glia (arrowhead).

### DETAILED DESCRIPTION OF THE INVENTION

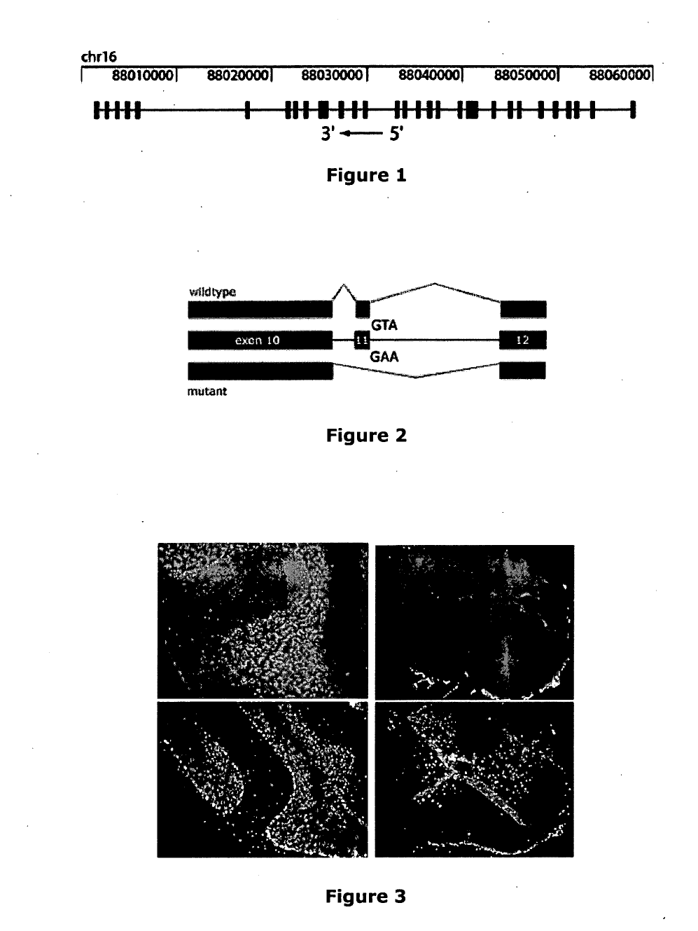

In the present invention, the Sensin gene has been identified in the genomes of many organisms, all of which encode Sensin proteins of between 1500 to 1800 amino acids that contain a N-terminal lysine-rich domains and a C-terminal zinc finger domain that bears similarity to the RING finger subfamily. The mouse Sensin gene locus is located on chromosome 16 and contains at least 30 exons (FIG. 1).

In addition, a mouse strain exhibiting adult-onset motor-dysfunction was identified from a library of ENU mutagenized mice. This strain carries a point mutation in the Sensin gene. The mutation occurs in a sequence that encodes a well-conserved splice donor site following exon 11 of the Sensin gene, leading to aberrant splicing. This process creates a distinct species of Sensin transcript that is missing the exon preceding the mutation (FIG. 2). This transcript is predicted to yield a mutant protein lacking 14 amino acids that may be important for gene function. Preliminary analysis also suggests there is lower than wildtype expression of the senseless gene as assessed by RT-PCR of mutant brain and spinal cord RNA.

Mice with two copies of a chemically-induced mutation in the Sensin gene are born at an expected rate and exhibit normal growth and behavior until near three to four months of age. Initial ataxic gait rapidly degenerates into failure to right and titanic paralysis. Resultant impaired ability to feed leads to visually evident weight loss and eventual death. These mice are possibly impaired in pain response as assayed by tail pinch. Muscle tone appears grossly normal in Sensin-mutant mice. Mice heterozygous for this mutant allele appear healthy and normal.

Sensin Sequences

Wild type mouse Sensin nucleic acid has the following sequence (SEQ ID NO: 1) (start and stop codons are underlined):

CTTCGGCTCTGCAGACTGAGAGCCGGGCTGCCACCGCCACCATGGGCGGGAAGAACAAGCAGCGGACTAA
GGGGAACCTCAGGCCTTCAAACAGTGGCCGAGCTGCAGAACTCCTCGCCAAAGAGCAGGGAACAGTGCCT
GGGTTCATTGGTTTTGGCACATCTCACAGTGACCTGGGCTATGTTCCGGCTGTTCAAGGCGCGGAAGACA
TAGACAGTCTTGTAGATTCCGATTTCCGAATGGTGCTGCGGAAACTTTCCAAAAAAGATGTTACAACAAA
GCTAAAGGCAATGCAAGAATTTGGAATTATGTGCACAGAGAGAGACACAGAAGCCGTCAAAGGGGTTCTT
CCATACTGGCCAAGAATCTTCTGCAAAATCTCCCTTGATCATGATCGCCGTGTTCGAGAGGCGACGCAGC
AAGCTTTTGAAAAACTTATCCTTAAAGTAAAGAAGCACTTAGCTCCCTATTTAAAAAGCGTGATGGGCTA
TTGGTTGATGGCTCAGTGTGACACATATCCACCAGCTGCACTGGCAGCAAAAGATGCATTCGAAGCCGCT
TTCCCTCCAAGCAAGCAACCTGAAGCCATAGCGTTTTGCAAGGAAGAAATTACAACTGTGTTACAGGACC
ATCTTCTGAAGGAGACTCCTGACACACTCAGTGACCCTCAAACTGTGCCAGAGGAAGAGAGAGAGGCTAA
GTTCCACCGAGTCGTGACGTGCTCTTTATTGGCACTGAAGAGATTGCTTTGTTTCTTACCTAACAATGAG
CTTGATTCTCTGGAGGAGAAATTTAAATCACTTTTATCACAGAATAAATTTTGGAAGTATGGAAAACACA
GTGTACCTCAGGTCCGCTCAGCATATTTTGAGTTAGTTTCTGCTTTGTGCCAGCACGTTCCCCAGGTGAT
GAAAGAGGAAGCTGCCAAAGTGAGTCCATCTGTCCTGCTCAGCATTGATGACAGTGACCCTGTGGTCTGC
CCAGCTCTCTGGGAGGCTGTGCTCTACACGCTGACAACTATTGAGGACTGTTGGTTTCATGTAAATGCCA
AGAAAAGTGTGTTTCCGAAGCTGATGGCCATGATCCGAGAGGGTGGCCGGGGCCTAGCTGCTGTTATGTA
TCCTTACCTCTTACCGTTCATCAGCAAACTCCCTCAGTCCATCACAGAGCCAAAGCTGGACTTCTTCAAA
AACTTCCTCACCTCTCTAGTTACCGGGCTGTCAACTGAGAGGACCAAATCGAGCTCTTCTGAGTGCTCAG
CTGTCATCCCGGCATTTTTTGAATGTTTGCGGTTTATAATGCAGCAGAACTTAGGCGAGGAGGAGATGGT
GCAGATGCTTATCAATGAGCAGTTGATCCCATTTATTGATACAGTCCTCAAAGACTCAGGACTGCACCAC
GGACCGATGTTTGACCATTTAGCAGACACATTGAGCTCTTGGGAAGCCAAAGCAGATGCAGAGCGAGACC
CTGGAGCTGTTTACAACTTGGAGAACGTTCTGCTAAGCTTCTGGGGAAGACTGTCAGAGATCTGCACCGA
GAAAATCCGCCAGCCAGAAGCAGATGTGAAGTCCGTTCTGTGTGTGTCTAGCCTAGTAGGGGTCCTTCAG
AGGCCAAGGAGCTCACTGGAGTTACACAGAAAGAAAACTGCTCAGGTCAGGTTTGCCATCAATATACCTG
AAGCTCATAAAGGGGATGAAAAGTCCATGTCTTCAGAAGGAGAGAACAGCGAGGGCTCTGACGGAGGGGC
TCAGTCTCCTCTCAGTAATACATCTTCAGACTTAGTATCTCCTTTAAGGAAAAAACCTCTGGAAGACTTA
GTCTGTAAGCTGGCAGAGGTGAGCATTAGCTTTGTCAACGAGCGGAAGTCAGAGCAGCACCTGCAGTTTC
TCTCCACGCTGCTCGACTCCTTCTCCTCCGTCCAAGTATTTAACATTCTCCTCAGTGACAAACAGAAGAA
TGTCGTCAAAGCCAAACCTCTGGAAATAACCAAACTTGCAGAAAAAAATCCTGCAGTTAAGTTCTTATAT
CATAAATTGATAGGCTGGCTAAATGACAGCCAGAAAGAGGACGGGGGCTTCCTGGTGGACATCTTGTATA
GTGCCCTCCGATGCTGTGACAGTGGTGTGGAAAGGAAAGAAGTCTTGGATGATCTAACCAAGGAGGACCT
GAAGTGGAGTTCTCTTCTTCAGGTCATTGAAAAGGCATGTTCTAGCTCAGATAAACATGCTTTAGTAACT
CCTTGGCTAAAAGGCAGTATTCTTGGAGAGAAATTGGTTGCCTTGGCAGACTGTCTTTGTGATAAGGACT
TGGAAGCCACAACATCTGAATCCCACTCATCAGAACAGTGGAGTCTGCTAAGACTGGCGTTATCCCAACA
TGTCAAAAACGATTACTTGATTGGAGAAGTATACGTTGGAAGAATTATTGTTAAACTTCATGAAACTTTA
TCTAAAACAAAGGATTTATCAGAAGCGGCAAACAGTGACTCTTCAGTGTCTTTTGTCTGTGATGTGGTCC
ATAGCTTCTTCAGTTCAGCAGGAGGAGGCTTGCTAATGCCACCATCTGAAGACTTGTTATTAACTCTCTT
TCAGTTATGCGCTCAGAGCAAAGAACGGACACACTTGCCAGATTTTCTGATCTGTAAACTGAAGAATACT
TTGCTTTCTGGTGTAAATTTATTGGTCCATCAAACTGCAAGTACATATGAGCAGAGTACCTTCCTACGTT
TGTCTGTTCTGTGGCTGAAGGACCAGGTTCAGTCTTCGGCTTTGGATAACACAAGTCTTCAGGTCCTCTT
GTCTGCTGCTGGCGACTTGCTGGGTACTCTTGTAGAGAGTGAGGACACGTCTCTTCTTGGAGTTTATATC
GGAAGTGTAATGCCCAGTGACAGCGAGTGGGAAAAGATGAGGCAGGCTCTTCCTGTTCAGTGGTTACACA
GACCTCTTTTAGAGGGAAGGCTGAGTTTAAATTATGAATGCTTCAAGACAGATTTTAAGGAACAAGACAC
AAAGACTCTTCCCAACCATCTGTGTACTTCATCGTTATTGAGCAAAATGATCTTAGTTGCCCAAAAAAAG
AAATTAGTCCTAGAAGACAACGTCCTTGAAAAAATAATTGCAGAGCTGCTGTATTCACTGCAGTGGTGTG
AAGAACTCGACAATGCGCCATCTTTCCTAAGTGGATTTTGTGGAATCCTTCAAAAAATGAATATAACTTA
CAGCAACTTATCTGTACTTAGTGAGACTTCTAGCCTCTTGCAGCTGTTATTTGACAGATCTAGAAAAAAT
GGCACACTTTGGTCTCTTATTATTGCTAAGTTGATCCTTTCCCGAAGTATTTCATCTGATGAAGTGAAAC
CATACTATAAAAGAAAAGAAAGTTTCTTCCCTCTAACAGAAGGTAGTTTGCATACCATTCAAAGTCTCTG
TCCATTCTTGTCTAAAGAAGAGAAGAAGGAATTTAGTGCTCACAGTATACCCGCTTTTTTGGGTTGGACT
AAAGAAGACCTTTGCAGTATTAATGGAGCCTTTGGACATCTTGCCATTTTCAATTCTTGTCTGCAAACCA
GAAGTATAGACGACAAACAGCTATTGCATGGCATATTAAAAATTATAACAAGCTGGAGGAAACAGCATGA
AGATATTTTTCTTTTTAGCTGTAATCTGTCGGAAGCGAGTCCAGAGGTCCTTGGTTTAAACATAGAGATC
ATGCGGTTCCTTTCCTTGTTTCTCAAGCACTGCGCGTACCCGCTCCCGCTGGCAGACAGTGAATGGGACT
TCATCATGTGCTCCATGCTGGCTTGGTTGGAGACAACCAGTGAGAACCAGGCTCTGTACTCTGTTCCACT
TGTGCAGCTGTTTGCCTGTGTCAGCTTTGATCTGGCCTGTGATCTCTGTGCCTTTTTTGACTCAATAACT
CCAGATATTGTTGACAATCTTCCTGTAAATCTCATCAGTGAGTGGAAAGAGTTTTTTTCTAAAGGCATCC
ACAGTTTGCTATTACCTCTTTTGGTAAATGCTATCGGAGAAAACAAAGACCTATCTGAAACGTCCTTTCA
GAACGCAATGCTGAAACCCATGTGTGAAACACTAACATACATCTCCAAGGACCAGCTACTGAGCCACAAG
CTCCCTGCGAGATTGGTTGCCAGCCAGAAAACAAACTTGCCAGAGCACCTCCAGACTCTGCTGAACACTT
TGACCCCACTGCTTCTCTTCAGAGCCAGACCTGTGCAAATTGCTGCTTATCATATGCTGTGCAAACTGAT
GCCTGAATTGCCACAGCATGATCAGGACAATCTGAGGTCGTATGGAGATGAAGAGGAAGAACCAGCCTTG
TCGCCGCCGGCCGCGCTGATGTCTCTCCTCAGCTCTCAGGAGGAGCTGCTGGAGAATGTCCTGGGCTGTG
TCCCTGTGGGCCAGATCGTGACCGTTAAGCCACTGAGCGAGGACTTCTGCTATGTCCTGGGATACCTCCT
CACTTGGAAGTTAATACTGACTTTCTTCAAAGCTGCATCGTCTCAGCTTCGTGCTCTGTATTCAATGTAC
CTTCGGAAAACAAAGAGTCTGAATAAATTACTCTATCATCTCTTCAGACTTATGCCAGAAAACCCTACGT
ACGGAGAGACAGCTATTGAGGTATCAAGTAAAGACCCCAAGACCTTCTTCACCGAGGAGGTTCAGCTGAG
TATTAGAGAAACAGCAACTCTTCCGTATCATATCCCACACCTGGCGTGCTCGGTCTATCACATGACTTTA
AAAGACTTGCCTGCCATGGTTAGGCTATGGTGGAATAGCAGTGAGAAGCGTGTCTTCAATATTGTAGATA
GATTTACAAGCAAGTATGTCAGCAATGTTCTTTCTTTTCAAGAAATATCTTCTGTACAAACAAGTACACA
GCTATTCAATGGCATGACGGTTAAGGCGCGAGCTACTACTCGAGAAGTGATGGCTACGTACACCATCGAA
GACATAGTCATTGAACTCATAATACAGTTGCCTTCCAATTACCCACTGGGCTCAATAACAGTGGAAAGTG
GGAAGAGGATCGGGGTGGCTGTGCAGCAGTGGCGAAACTGGATGCTGCAGCTGAGCACGTACCTCACTCA
CCAGAACGGAAGTATCATGGAAGGCTTAGCATTATGGAAGAATAATGTAGACAAACGGTTTGAAGGTGTT
GAAGATTGTATGATCTGCTTCTCAGTTATTCATGGTTTCAACTATTCTCTTCCCAAAAAAGCCTGTAGAA
CATGCAAGAAAAAGTTTCACTCAGCTTGCCTGTACAAATGGTTTACATCTAGCAACAAGTCCACTTGCCC
GCTCTGCCGTGAGACCTTTTTCTGAGGTTTTTTTCATTGGAAGTTGTCGCTGCCGTAGGTCAAGCCAAAG
GGAATGGATTGGCTCCACCTTGAAGTACTGATGTGAAGCCAGTGAGCATGACAAAGTGCCATCG

Wild type human Sensin nucleic acid has the following sequence (SEQ ID NO: 2):

CAGCAAATGGACAGGTGGGTGGCGGAAAAGGGCCCGGGGGAAGTTATTACAGGGTGTCCTCTTCCGCCGC
CAGAAGCCGGAAGTTGTGTCCCGGACGTGTCAACCGGGGTCTGAGTGCTCAGAGTACAGCTGCAACCGCG
ACCATGGGCGGGAAGAACAAGCAGCGAACTAAAGGGAACCTGAGGCCTTCAAACAGTGGCCGAGCTGCAG
AACTCCTTGCCAAAGAACAGGGAACAGTGCCTGGATTTATTGGTTTTGGAACATCTCAGAGTGACCTAGG
CTATGTTCCTGCTATTCAAGGAGCTGAAGAAATTGACAGTCTTGTAGATTCTGATTTCCGAATGGTGCTG
CGGAAACTTTCAAAGAAAGATGTCACCACAAAATTAAAAGCTATGCAGGAATTTGGAACCATGTGTACAG
AGAGAGACACAGAAACTGTGAAAGGAGTTCTTCCATATTGGCCAAGAATTTTTTGCAAAATTTCACTTGA
TCATGACCGTCGCGTCCGAGAAGCCACACAACAAGCTTTTGAAAAACTTATCCTTAAAGTAAAGAAACAG
TTGGCTCCCTACTTAAAAAGTTTAATGGGATATTGGCTAATGGCTCAGTGTGATACTTACACACCAGCTG
CGTTTGCAGCAAAAGATGCATTTGAAGCGGCTTTTCCTCCAAGCAAGCAACCTGAAGCCATAGCATTTTG
TAAGGATGAAATTACAAGTGTGCTGCAGGATCATCTTATAAAAGAAACACCTGATACACTCAGTGACCCG
CAAACTGTTCCAGAGGAAGAAAGAGAAGCTAAATTCTACCGGGTTGTAACTTGTTCCTTATTGGCATTAA
AGAGATTACTTTGCCTTTTACCTGATAATGAGCTTGATTCTCTGGAGGAGAAATTTAAGTCTCTTTTATC
ACAGAATAAGTTTTGGAAGTATGGAAAACACAGTGTACCTCAGATTCGCTCAGCTTATTTTGAGTTAGTC
TCTGCATTGTGCCAGCGCATTCCACAGTTGATGAAAGAGGAAGCATCCAAAGTGAGCCCATCAGTTCTAC
TTAGCATTGATGACAGTGACCCAATTGTCTGCCCAGCTCTCTGGGAAGCTGTACTCTATACACTTACAAC
TATTGAGGACTGTTGGCTTCATGTAAATGCAAAAAAGAGTGTGTTTCCCAAGCTATCAACTGTGATTCGT
GAAGGTGGTCGGGGTCTAGCTACTGTCATATATCCTTACCTTCTGCCATTCATCAGCAAGCTCCCTCAGT
CCATCACAAATCCAAAGTTGGATTTCTTCAAAAATTTCCTCACGTCTCTAGTTGCTGGGCTGTCAACAGA
GAGAACTAAAACCAGCTCTTTAGAGTCCTCGGCAGTAATATCTGCTTTTTTTGAATGCTTACGTTTTATA
ATGCAGCAAAACTTAGGTGAGGAAGAGATTGAACAGATGCTCGTCAATGATCAGTTGATCCCTTTTATTG
ATGCAGTTCTCAAAGACCCAGGATTGCAACATGGGCAGCTATTTAACCATTTAGCAGAAACTCTAAGTTC
CTGGGAAGCCAAAGCAGACACGGAAAAAGATGAAAAAACAGCTCACAACTTGGAGAACGTACTGATACAT
TTCTGGGAAAGACTGTCAGAGATCTGTGTTGCGAAAATCAGTGAGCCAGAAGCTGATGTTGAGTCCGTTT
TGGGTGTATCTAACCTATTACAGGTGCTTCAGAAGCCGAAGAGCTCATTGAAGTCAAGTAAAAAAAAAAA
TGGTAAGGTTAGATTTGCTGATGAGATACTTGAAAGCAATAAAGAGAATGAAAAATGTGTATCTTCAGAA
GGAGAGAAGATTGAAGGCTGGGAATTAACAACTGAACCTTCTCTCACTCATAATTCTTCAGGCCTTTTGT
CTCCTCTAAGGAAAAAACCTTTGGAAGACTTAGTCTGTAAACTCGCAGATATAAGTATTAATTATGTCAA
TGAACGAAAGTCAGAGCAACATCTAAGGTTTCTTTCTACTCTGCTTGACTCCTTTTCTTCAAGCCGAGTA
TTTAAAATGCTACTTGGTGATGAAAAACAGAGTATTGTCCAAGCCAAACCTCTTGAAATAGCCAAGCTTG
TACAAAAAAATCCTGCGGTGCAGTTTTTATACCAGAAACTGATAGGTTGGCTAAATGAAGATCAAAGGAA
GGATTTTGGTTTCCTGGTGGACATTTTGTACAGTGCTCTCCGGTGCTGTGACAATGATATGGAAAGAAAA
AAAGTCTTGGATGATCTAACCAAGGTGGACTTGAAATGGAATTCTCTTCTTAAGATTATTGAAAAGGCAT
GTCCTAGTTCAGATAAACATGCTTTAGTAACTCCTTGGCTCAAAGGCGATATCCTTGGTGAGAAATTGGT
CAACTTGGCAGATTGTCTTTGTAATGAGGACTTGGAATCCAGGGTATCTTCAGAATCTCACTTCTCAGAA
AGATGGACTCTTCTAAGCTTGGTATTATCCCAACATGTTAAAAATGATTACTTGATTGGAGACGTATATG
TTGAAAGAATCATTGTTAGACTTCATGAAACTTTATTCAAAACAAAGAAATTATCAGAAGCTGAAAGCAG
TGACTCATCAGTGTCTTTTATCTGTGATGTGGCCTATAACTATTTCAGCTCAGCGAAAGGATGCTTGCTA
ATGCCATCATCTGAAGATTTATTATTAACTCTCTTTCAGTTATGTGCTCAGAGCAAAGAAAAAACACATT
TGCCAGATTTTCTTATCTGTAAACTGAAAAATACTTGGCTCTCTGGTGTAAATTTATTGGTTCATCAAAC
TGACAGTTCATATAAAGAGAGTACCTTCCTACATTTGTCTGCTCTGTGGCTGAAGAACCAAGTTCAGGCT
TCATCTTTGGATATCAACAGTCTCCAAGTCCTCTTGTCTGCTGTTGATGATTTGCTAAATACACTTCTAG
AGAGTGAAGATTCTTATCTTATGGGAGTTTATATTGGAAGTGTAATGCCGAACGACAGTGAATGGGAAAA
GATGAGGCAGTCTCTTCCTATGCAGTGGTTACATAGACCTCTTTTAGAGGGAAGATTGAGTTTGAATTAT
GAATGTTTCAAAACAGATTTTAAGGAACAGGACATAAAGACACTTCCCAGCCATTTGTGTACTTCAGCAT
TATTGAGCAAAATGGTCTTAATTGCACTGAGAAAGGAAACAGTCTTAGAAAATAATGAGCTTGAGAAAAT
AATTGCAGAACTGCTTTATTCACTGCAGTGGTGTGAAGAATTAGATAACCCACCTATTTTTCTAATTGGA
TTTTGTGAAATACTTCAAAAAATGAATATTACGTATGATAACTTACGTGTACTTGGTAATACGTCGGGCC
TTTTGCAGCTGTTATTTAACAGGTCCAGAGAACATGGCACACTGTGGTCTCTTATTATTGCTAAGTTGAT
CCTTTCCCGAAGCATTTCATCTGATGAAGTAAAACCACATTATAAGAGAAAAGAAAGTTTTTTTCCACTA
ACTGAAGGCAATTTGCATACCATTCAAAGTCTATGTCCATTTTTGTCAAAAGAAGAAAAGAAAGAATTTA
GTGCTCAATGTATACCTGCTCTTTTGGGCTGGACTAAGAAAGATCTTTGCAGCACTAATGGAGGTTTTGG
ACATCTTGCCATTTTCAATTCTTGTCTGCAAACCAAAAGTATAGATGATGGAGAGCTATTACATGGAATA
TTAAAAATCATAATATCCTGGAAGAAAGAGCATGAAGATATTTTTCTTTTCAGTTGTAATCTATCAGAAG
CAAGTCCAGAGGTACTGGGTGTAAATATAGAAATAATCCGGTTTCTTTCCCTATTTCTGAAATACTGCTC
ATCCCCTTTGGCAGAGAGTGAGTGGGACTTCATCATGTGCTCCATGTTGGCTTGGTTGGAGACAACAAGT
GAGAATCAGGCATTGTATTCTATTCCACTTGTGCAACTGTTTGCCTGTGTCAGCTGTGATTTGGCCTGTG
ACCTCAGTGCTTTCTTTGATTCCACAACTCTGGATACCATTGGCAATCTTCCTGTAAATCTAATCAGTGA
ATGGAAAGAATTTTTTTCCCAAGGCATCCACAGTTTGCTTTTACCTATTTTGGTGACTGTTACAGGAGAA
AACAAAGATGTGTCTGAAACATCCTTTCAGAATGCAATGCTGAAACCCATGTGTGAAACATTAACGTATA
TCTCAAAGGAACAGCTATTGAGTCACAAACTTCCTGCAAGATTAGTTGCTGACCAAAAAACAAACTTACC
AGAATATCTCCAGACTTTGTTAAATACATTGGCCCCATTACTCCTCTTCAGAGCTAGGCCTGTGCAAATT
GCTGTTTATCATATGCTATACAAATTGATGCCTGAATTACCACAGTATGATCAGGATAATCTAAAGTCAT
ACGGAGATGAAGAAGAAGAGCCAGCCTTGTCACCACCAGCAGCACTGATGTCTCTTCTTAGCATTCAAGA
GGACTTACTAGAAAATGTTTTGGGGTGTATTCCTGTTGGACAGATAGTTACTATTAAACCACTGAGTGAA
GACTTCTGTTATGTTCTGGGATACCTTCTCACTTGGAAATTAATACTAACTTTCTTCAAAGCTGCATCAT
CACAGCTTCGGGCTTTGTATTCCATGTATCTTCGGAAAACAAAGAGTTTGAATAAATTGCTCTATCACCT
GTTCAGGCTTATGCCAGAAAATCCAACCTATGCAGAGACAGCAGTTGAGGTCCCAAATAAGGACCCTAAA
ACATTCTTTACTGAGGAGCTCCAGCTGAGTATTAGAGAAACAACAATGCTTCCATACCACATTCCACACT
TGGCTTGTTCAGTCTATCATATGACATTAAAAGACTTGCCTGCCATGGTTAGGTTGTGGTGGAATAGCAG
TGAGAAGCGTGTTTTCAATATTGTGGATAGATTTACAAGCAAGTATGTCAGCAGTGTTCTTTCTTTTCAA
GAAATATCTTCTGTACAAACAAGTACACAACTATTTAATGGCATGACGGTTAAAGCTCGAGCTACTACTC
GAGAGGTAATGGCTACTTATACTATTGAGGACATAGTTATTGAACTTATAATACAACTGCCTTCAAATTA
TCCACTGGGTTCAATAATAGTAGAAAGTGGGAAAAGAGTAGGAGTAGCTGTTCAGCAGTGGCGGAACTGG
ATGCTGCAGTTAAGCACTTACCTCACCCATCAGAATGGAAGTATTATGGAAGGCTTAGCTTTATGGAAAA
ATAACGTAGACAAACGTTTTGAGGGTGTTGAAGATTGCATGATCTGTTTCTCAGTCATTCACGGTTTCAA
CTATTCCCTTCCCAAAAAAGCCTGTAGAACATGCAAGAAAAAATTCCATTCAGCCTGCTTGTACAAATGG
TTTACATCTAGCAACAAATCCACTTGTCCACTGTGTCGTGAGACGTTTTTCTGA

A preferred mutation deletes the following sequence from the wildtype mouse sensin polypeptide (SEQ ID NO: 3):
KEDLKWSSLLQVIE

A preferred mutation deletes the following sequence from the wildtype human sensin polypeptide (SEQ ID NO: 4):
KVDLKWNSLLKIIE

A preferred mutated mouse Sensin polypeptide has the following sequence (SEQ ID NO: 5). N-terminal lysine-rich domains are underlined, and RING domain homology is italicized:

MGGKNKQRTKGNLRPSNSGRAAELLAKEQGTVPGFIGFGTSHSDLGYVPAVQGAEDIDSLVDSDFRMVLR
KLSKKDVTTKLKAMQEFGIMCTERDTEAVKGVLPYWPRIFCKISLDHDRRVREATQQAFEKLILKVKKHL
APYLKSVMGYWLMAQCDTYPPAALAAKDAFEAAFPPSKQPEAIAFCKEEITTVLQDHLLKETPDTLSDPQ
TVPEEEREAKFHRVVTCSLLALKRLLCFLPNNELDSLEEKFKSLLSQNKFWKYGKHSVPQVRSAYFELVS
ALCQHVPQVMKEEAAKVSPSVLLSIDDSDPVVCPALWEAVLYTLTTIEDCWFHVNAKKSVFPKLMAMIRE
GGRGLAAVMYPYLLPFISKLPQSITEPKLDFFKNFLTSLVTGLSTERTKSSSSECSAVIPAFFECLRFIM
QQNLGEEEMVQMLINEQLIPFIDTVLKDSGLHHGPMFDHLADTLSSWEAKADAERDPGAVYNLENVLLSF
WGRLSEICTEKIRQPEADVKSVLCVSSLVGVLQRPRSSLELHRKKTAQVRFAINIPEAHKGDEKSMSSEG
ENSEGSDGGAQSPLSNTSSDLVSPLRKKPLEDLVCKLAEVSISFVNERKSEQHLQFLSTLLDSFSSVQVF
NILLSDKQKNVVKAKPLEITKLAEKNPAVKFLYHKLIGWLNDSQKEDGGFLVDILYSALRCCDSGVERKE
VLDDLTKACSSSDKHALVTPWLKGSILGEKLVALADCLCDKDLEATTSESHSSEQWSLLRLALSQHVKND
YLIGEVYVGRIIVKLHETLSKTKDLSEAANSDSSVSFVCDVVHSFFSSAGGGLLMPPSEDLLLTLFQLCA
QSKERTHLPDFLICKLKNTLLSGVNLLVHQTASTYEQSTFLRLSVLWLKDQVQSSALDNTSLQVLLSAAG
DLLGTLVESEDTSLLGVYIGSVMPSDSEWEKMRQALPVQWLHRPLLEGRLSLNYECFKTDFKEQDTKTLP
NHLCTSSLLSKMILVAQKKKLVLEDNVLEKIIAELLYSLQWCEELDNAPSFLSGFCGILQKMNITYSNLS
VLSETSSLLQLLFDRSRKNGTLWSLIIAKLILSRSISSDEVKPYYKRKESFFPLTEGSLHTIQSLCPFLS
KEEKKEFSAHSIPAFLGWTKEDLCSINGAFGHLAIFNSCLQTRSIDDKQLLHGILKIITSWRKQHEDIFL
FSCNLSEASPEVLGLNIEIMRFLSLFLKHCAYPLPLADSEWDFIMCSMLAWLETTSENQALYSVPLVQLF
ACVSFDLACDLCAFFDSITPDIVDNLPVNLISEWKEFFSKGIHSLLLPLLVNAIGENKDLSETSFQNAML
KPMCETLTYISKDQLLSHKLPARLVASQKTNLPEHLQTLLNTLTPLLLFRARPVQIAAYHMLCKLMPELP
QHDQDNLRSYGDEEEEPALSPPAALMSLLSSQEELLENVLGCVPVGQIVTVKPLSEDFCYVLGYLLTWKL
ILTFFKAASSQLRALYSMYLRKTKSLNKLLYHLFRLMPENPTYGETAIEVSSKDPKTFFTEEVQLSIRET
ATLPYHIPHLACSVYHMTLKDLPAMVRLWWNSSEKRVFNIVDRFTSKYVSNVLSFQEISSVQTSTQLFNG
MTVKARATTREVMATYTIEDIVIELIIQLPSNYPLGSITVESGKRIGVAVQQWRNWMLQLSTYLTHQNGS
IMEGLALWKNNVDKRFEGVEDCMICFSVIHGFNYSLPKKACRTCKKKFHSACLYKWFTSSNKSTCPLCRE
TFF

A preferred mutated human Sensin polypeptide has the following sequence (SEQ ID NO: 6). N-terminal lysine-rich domains are underlined, and RING domain homology is italicized:

MGGKNKQRTKGNLRPSNSGRAAELLAKEQGTVPGFIGFGTSQSDLGYVPAIQGAEEIDSLVDSDFRMVLR
KLSKKDVTTKLKAMQEFGTMCTERDTETVKGVLPYWPRIFCKISLDHDRRVREATQQAFEKLILKVKKQL
APYLKSLMGYWLMAQCDTYTPAAFAAKDAFEAAFPPSKQPEAIAFCKDEITSVLQDHLIKETPDTLSDPQ
TVPEEEREAKFYRVVTCSLLALKRLLCLLPDNELDSLEEKFKSLLSQNKFWKYGKHSVPQIRSAYFELVS
ALCQRIPQLMKEEASKVSPSVLLSIDDSDPIVCPALWEAVLYTLTTIEDCWLHVNAKKSVFPKLSTVIRE
GGRGLATVIYPYLLPFISKLPQSITNPKLDFFKNFLTSLVAGLSTERTKTSSLESSAVISAFFECLRFIM
QQNLGEEEIEQMLVNDQLIPFIDAVLKDPGLQHGQLFNHLAETLSSWEAKADTEKDEKTAHNLENVLIHF
WERLSEICVAKISEPEADVESVLGVSNLLQVLQKPKSSLKSSKKKNGKVRFADEILESNKENEKCVSSEG
EKIEGWELTTEPSLTHNSSGLLSPLRKKPLEDLVCKLADISINYVNERKSEQHLRFLSTLLDSFSSSRVF
KMLLGDEKQSIVQAKPLEIAKLVQKNPAVQFLYQKLIGWLNEDQRKDFGFLVDILYSALRCCDNDMERKK
VLDDLTKACPSSDKHALVTPWLKGDILGEKLVNLADCLCNEDLESRVSSESHFSERWTLLSLVLSQHVKN
DYLIGDVYVERIIVRLHETLFKTKKLSEAESSDSSVSFICDVAYNYFSSAKGCLLMPSSEDLLLTLFQLC
AQSKEKTHLPDFLICKLKNTWLSGVNLLVHQTDSSYKESTFLHLSALWLKNQVQASSLDINSLQVLLSAV
DDLLNTLLESEDSYLMGVYIGSVMPNDSEWEKMRQSLPMQWLHRPLLEGRLSLNYECFKTDFKEQDIKTL
PSHLCTSALLSKMVLIALRKETVLENNELEKIIAELLYSLQWCEELDNPPIFLIGFCEILQKMNITYDNL
RVLGNTSGLLQLLFNRSREHGTLWSLIIAKLILSRSISSDEVKPHYKRKESFFPLTEGNLHTIQSLCPFL
SKEEKKEFSAQCIPALLGWTKKDLCSTNGGFGHLAIFNSCLQTKSIDDGELLHGILKIIISWKKEHEDIF
LFSCNLSEASPEVLGVNIEIIRFLSLFLKYCSSPLAESEWDFIMCSMLAWLETTSENQALYSIPLVQLFA
CVSCDLACDLSAFFDSTTLDTIGNLPVNLISEWKEFFSQGIHSLLLPILVTVTGENKDVSETSFQNAMLK
PMCETLTYISKEQLLSHKLPARLVADQKTNLPEYLQTLLNTLAPLLLFPARPVQIAVYHMLYKLMPELPQ
YDQDNLKSYGDEEEEPALSPPAALMSLLSIQEDLLENVLGCIPVGQIVTIKPLSEDFCYVLGYLLTWKLI
LTFFKAASSQLRALYSMYLRKTKSLNKLLYHLFRLMPENPTYAETAVEVPNKDPKTFFTEELQLSIRETT
MLPYHIPHLACSVYHMTLKDLPAMVRLWWNSSEKRVFNIVDRFTSKYVSSVLSFQEISSVQTSTQLFNGM
TVKARATTREVMATYTIEDIVIELIIQLPSNYPLGSIIVESGKRVGVAVQQWRNWMLQLSTYLTHQNGSI
MEGLALWKNNVDKRFEGVEDCMICFSVIHGFNYSLPKKACRTCKKKFHSACLYKWFTSSNKSTCPLCRET
FF

Wild-type mouse Sensin polypeptide has the following sequence (SEQ ID NO. 7). N-terminal lysine-rich domains are underlined, and RING domain homology is italicized:

>consensus_mouse_protein
MGGKNKQRTKGNLRPSNSGRAAELLAKEQGTVPGFIGFGTSHSDLGYVPAVQGAEDIDSLVDSDFRMVLR
KLSKKDVTTKLKAMQEFGIMCTERDTEAVKGVLPYWPRIFCKISLDHDRRVREATQQAFEKLILKVKKHL
APYLKSVMGYWLMAQCDTYPPAALAAKDAFEAAFPPSKQPEAIAFCKEEITTVLQDHLLKETPDTLSDPQ
TVPEEEREAKFHRVVTCSLLALKRLLCFLPNNELDSLEEKFKSLLSQNKFWKYGKHSVPQVRSAYFELVS
ALCQHVPQVMKEEAAKVSPSVLLSIDDSDPVVCPALWEAVLYTLTTIEDCWFHVNAKKSVFPKLMAMIRE
GGRGLAAVMYPYLLPFISKLPQSITEPKLDFFKNFLTSLVTGLSTERTKSSSSECSAVIPAFFECLRFIM
QQNLGEEEMVQMLINEQLIPFIDTVLKDSGLHHGPMFDHLADTLSSWEAKADAERDPGAVYNLENVLLSF
WGRLSEICTEKIRQPEADVKSVLCVSSLVGVLQRPRSSLELHRKKTAQVRFAINIPEAHKGDEKSMSSEG
ENSEGSDGGAQSPLSNTSSDLVSPLRKKPLEDLVCKLAEVSISFVNERKSEQHLQFLSTLLDSFSSVQVF
NILLSDKQKNVVKAKPLEITKLAEKNPAVKFLYHKLIGWLNDSQKEDGGFLVDILYSALRCCDSGVERKE
VLDDLTKEDLKWSSLLQVIEKACSSSDKHALVTPWLKGSILGEKLVALADCLCDKDLEATTSESHSSEQW
SLLRLALSQHVKNDYLIGEVYVGRIIVKLHETLSKTKDLSEAANSDSSVSFVCDVVHSFFSSAGGGLLMP
PSEDLLLTLFQLCAQSKERTHLPDFLICKLKNTLLSGVNLLVHQTASTYEQSTFLRLSVLWLKDQVQSSA
LDNTSLQVLLSAAGDLLGTLVESEDTSLLGVYIGSVMPSDSEWEKMRQALPVQWLHRPLLEGRLSLNYEC
FKTDFKEQDTKTLPNHLCTSSLLSKMILVAQKKKLVLEDNVLEKIIAELLYSLQWCEELDNAPSFLSGFC
GILQKMNITYSNLSVLSETSSLLQLLFDRSRKNGTLWSLIIAKLILSRSISSDEVKPYYKRKESFFPLTE
GSLHTIQSLCPFLSKEEKKEFSAHSIPAFLGWTKEDLCSINGAFGHLAIFNSCLQTRSIDDKQLLHGILK
IITSWRKQHEDIFLFSCNLSEASPEVLGLNIEIMRFLSLFLKHCAYPLPLADSEWDFIMCSMLAWLETTS
ENQALYSVPLVQLFACVSFDLACDLCAFFDSITPDIVDNLPVNLISEWKEFFSKGIHSLLLPLLVNAIGE
NKDLSETSFQNAMLKPMCETLTYISKDQLLSHKLPARLVASQKTNLPEHLQTLLNTLTPLLLFRARPVQI
AAYHMLCKLMPELPQHDQDNLRSYGDEEEEPALSPPAALMSLLSSQEELLENVLGCVPVGQIVTVKPLSE
DFCYVLGYLLTWKLILTFFKAASSQLRALYSMYLRKTKSLNKLLYHLFRLMPENPTYGETAIEVSSKDPK
TFFTEEVQLSIRETATLPYHIPHLACSVYHMTLKDLPAMVRLWWNSSEKRVFNIVDRFTSKYVSNVLSFQ
EISSVQTSTQLFNGMTVKAPATTREVMATYTIEDIVIELIIQLPSNYPLGSITVESGKRIGVAVQQWRNW
MLQLSTYLTHQNGSIMEGLALWKNNVDKRFEGVEDCMICFSVIHGFNYSLPKKACRTCKKKFHSACLYKW
FTSSNKSTCPLCRETFF

Wild type human Sensin polypeptide has the following sequence (SEQ ID NO: 8). N-terminal lysine-rich domains are underlined, and RING domain homology is italicized:

MGGKNKQRTKGNLRPSNSGRAAELLAKEQGTVPGFIGFGTSQSDLGYVPAIQGAEEIDSLVDSDFRMVLR
KLSKKDVTTKLKAMQEFGTMCTERDTETVKGVLPYWPRIFCKISLDHDRRVREATQQAFEKLILKVKKQL
APYLKSLMGYWLMAQCDTYTPAAFAAKDAFEAAFPPSKQPEAIAFCKDEITSVLQDHLIKETPDTLSDPQ
TVPEEEREAKFYRVVTCSLLALKRLLCLLPDNELDSLEEKFKSLLSQNKFWKYGKHSVPQIRSAYFELVS
ALCQRIPQLMKEEASKVSPSVLLSIDDSDPIVCPALWEAVLYTLTTIEDCWLHVNAKKSVFPKLSTVIRE
GGRGLATVIYPYLLPFISKLPQSITNPKLDFFKNFLTSLVAGLSTERTKTSSLESSAVISAFFECLRFIM
QQNLGEEEIEQMLVNDQLIPFIDAVLKDPGLQHGQLFNHLAETLSSWEAKADTEKDEKTAHNLENVLIHF
WERLSEICVAKISEPEADVESVLGVSNLLQVLQKPKSSLKSSKKKNGKVRFADEILESNKENEKCVSSEG
EKIEGWELTTEPSLTHNSSGLLSPLRKKPLEDLVCKLADISINYVNERKSEQHLRFLSTLLDSFSSSRVF
KMLLGDEKQSIVQAKPLEIAKLVQKNPAVQFLYQKLIGWLNEDQRKDFGFLVDILYSALRCCDNDMERKK
VLDDLTKVDLKWNSLLKIIEKACPSSDKHALVTPWLKGDILGEKLVNLADCLCNEDLESRVSSESHFSER
WTLLSLVLSQHVKNDYLIGDVYVERIIVRLHETLFKTKKLSEAESSDSSVSFICDVAYNYFSSAKGCLLM
PSSEDLLLTLFQLCAQSKEKTHLPDFLICKLKNTWLSGVNLLVHQTDSSYKESTFLHLSALWLKNQVQAS
SLDINSLQVLLSAVDDLLNTLLESEDSYLMGVYIGSVMPNDSEWEKMRQSLPMQWLHRPLLEGRLSLNYE
CFKTDFKEQDIKTLPSHLCTSALLSKMVLIALRKETVLENNELEKIIAELLYSLQWCEELDNPPIFLIGF
CEILQKMNITYDNLRVLGNTSGLLQLLFNRSREHGTLWSLIIAKLILSRSISSDEVKPHYKRKESFFPLT
EGNLHTIQSLCPFLSKEEKKEFSAQCIPALLGWTKKDLCSTNGGFGHLAIFNSCLQTKSIDDGELLHGIL
KIIISWKKEHEDIFLFSCNLSEASPEVLGVNIEIIRFLSLFLKYCSSPLAESEWDFIMCSMLAWLETTSE
NQALYSIPLVQLFACVSCDLACDLSAFFDSTTLDTIGNLPVNLISEWKEFFSQGIHSLLLPILVTVTGEN
KDVSETSFQNAMLKPMCETLTYISKEQLLSHKLPARLVADQKTNLPEYLQTLLNTLAPLLLFRARPVQIA
VYHMLYKLMPELPQYDQDNLKSYGDEEEEPALSPPAALMSLLSIQEDLLENVLGCIPVGQIVTIKPLSED
FCYVLGYLLTWKLILTFFKAASSQLRALYSMYLRKTKSLNKLLYHLFRLMPENPTYAETAVEVPNKDPKT
FFTEELQLSIRETTMLPYHIPHLACSVYHMTLKDLPAMVRLWWNSSEKRVFNIVDRFTSKYVSSVLSFQE
ISSVQTSTQLFNGMTVKARATTREVMATYTIEDIVIELIIQLPSNYPLGSIIVESGKRVGVAVQQWRNWM
LQLSTYLTHQNGSIMEGLALWKNNVDKRFEGVEDCMICFSVIHGFNYSLPKKACRTCKKKFHSACLYKWF
TSSNKSTCPLCRETFF

Wild type Drosophila Melanogaster Sensin polypeptide has the following sequence (SEQ ID NO: 9). N-terminal lysine-rich domains are underlined, and RING domain homology is italicized:

MGGKTKQAPRTKNNAKPSSSSRTAELLGSSTPIFVGFSAQTDGGGLVPFA
PGFASAEQMPDSFDAAISPQTQIILRKLSKKDPMTKKKALQELHELIEQS
DVEVLKNILPLWPKYYLNLASDPEHTVREQTQTVLQLLMAKCKKAMAPYL
KLLVPVWLGSRFDTYAPAASIASQSFRDTFAGNANRSREVCMHCQVEILE
YATRNLTFHTAATLSIGKSLTPEDAEQKYQRVIISSLKLLSFFMGQTAQT
EELSQVKEGFGTLVAHQKFWSFAKHKVPAIKAAWFECIYHILQSVALLDV
ITPQKTQLTNLCFQFIDDADPVVAPHIWGCVLLLQSNYVDWFVPLNIRKT
LLPKLSSLLQNGFNRNAQAICPNLLPFLSKVTQASLQDLDIYDFYQRFFD
DMKLAVTKKFDPPLSKSDCIVIHNAYFECLRFLMQQINNNKQREQKEEEF
SFSLLDNNVLEPIAWLLKSDSTHVKIFFQHSSALVAFWDRQINNRLDNGD
LYAKLLNKFWIRIFELVTQDLSAEEVNEQLLGHVLLLVQDLHMANPSLES
PSVKFVEGPNEKIEKSEPTTPVKKAQEAAAFIQKELKQLVIKLVRICLDK
ANKGSGSGTSSSRYIEQIRTLTKMFNDAAFYKSLTDDGDLASALNKFVSL
LGQLSCQACESVVEIVFEILPLLETGKRFEYIENTLMKLPQHGVQNLLLH
RLLSYPLCAEAAVRQMLSGPETCEMIARIAEEVVVDNDREKLNLLHKCFF
QTDTGDILINAKTVDKILLSMCGPLEQPVVDDAVEVCGSFIAQIMPVICS
NNNSSLHVRQHIFLKLFKFSLEHRPEDYLSEDTLWEITTCWQDGLSSKDI
EIDDDMLKCCAGIVEELANSAELKADTLDGMAEAMAKFVICSTENIEDEY
KRLERIDETLTALLETPLKTTDKVQQFENHCVLLEALHGSVTAGVPFENA
CLSRNEILPLLQRSTLNFSTIYKLVYQFPPPQDTNDPEDELTEDYCDPNA
DVLKKWNEPLIAELLQCIRVAGTAECWLEMSVLQSSTEELVLILSEKVQS
FMGNSSDLVAIVKERLQQAAVQQSSVIDCRLLSYLRFCPQYAAFEESASI
LLHEDLSENLVTQGALKTYVIALQFLLPKLSQKAITLSSAIMGTEPPEIW
VKAAVFHALLLNNFEGDVNEQTDRNIIVSAVQFMTSIGERQASQKDLLHY
NVEIQRQPYESVINTVEFIKLLTEVLKRFPYELSIKNWDAIRIGLSSWVL
SVSKSIAQYQDPKTSLFIVAVYELFAALIDFIRSEKQKSSTELLKNMIDE
WDSLFAKEVNLVLFKSYYLLTHEVSVDPGFQACYEALLEQITPVIERLDY
SFVYSFCKSNSNITLDHLCNFLFKQLYSVQHSVRLSAVHSLRQLTPHFVA
DDIELNEKQSESLDASTTICKWHFLNRFEDYLTRYDALITKYLEEFTFKL
SELDDLEPIDRHNALSYLFLWDCIINACAKSPVALRAVYTNWLNDNKYEE
NFLHFLFRAMPVDILKNHGAKVHSNGVYKELTWSQQKDRHLPLERYACHL
YTEVLRKLPAVVRRWWNATQSRQKNFIDNLTTNYVSSLICSEELKAIANR
KEKHENMQVTVHSSTREVLAVYAIDEARMELVITLAPNYPLGAVKVECGK
QIGGRASSRNVGMQLTIFLTHQNGTIYDGLTMWKNNLDKKFEGVEECYVC
YTVIHQETCQLPKLTCKTCKKKFHGPCLYKWFTTSSKSTCPICRNVF

Wild type Caenorhabditis elegans Sensin polypeptide has the following sequence (SEQ ID NO: 10). N-terminal lysine-rich domains are underlined, and RING domain homology is italicized:

MKICFFFPPKRAQKWQKKCFFPNVSAEINVASSNRLHQVVEIDDETRIVMKKLTKKDCQTREKGLKELME
LINTENSSIESSYEHFCGLVAQLTTDGSPTVRMLTMKVISQFLTKLKKSASKGLKKIIPFVLFAKSDVTN
GVAAAASAVIRDGFDADKKRQVVELFVPNTFDLAAKIAEGKHELSLPAEYDASEDLETRKMRLETQSLNT
FLSYIKEYGNESKLWEEPARKLFSNSEFIKKTFAGKKEALKVQLLNISYKFSDNIEVILSNPVISTYIQA
SLDAQTFSTECATAWEGILILLPDERFHAKCSLQKGIYPRLLNLIRKKGNHWRVLKHYLLPAVSVLLQKL
ENPALITSIITSFTDNLPWQAEASMNAIHCWFCTFSDFVKWILGNDRINLEILKDLSPLIVEMSNQSMHF
NTAEATECISGLIHWIIEKKVLENPAEFFDLLKTSIYEVAPPEKSRLFADSLTLPAKHLELAHLHGNLLS
NPDVDFHIIRNLARASNSEYFEETCRNINNFEFIENSDRFDMLQAVEIVKLIEMKPSLSLQIKNNHVGRQ
LLLSENSEIWEKSLKNVPAGVFQEMVNFWHEKRNGKAIAQAVNFLKKMGIQLDTNAAAENVDFLISLLQS
LDSKEDPEERKNLVLKLLSALFDAEDEPKLEHFESLKSHLNGDFEQFFEKLFANMEEEDAERVLEIAARF
DKLVGFCDADSRGEIAGKMILGRREFDEMSEKLHFLELDVLTVSQHTTIITDALSRPIEHLEEKEATKMV
KELGRLALFSVASNYNSSIHQLFAWQMIRVISALGNRYCLKFLDEELQQLRIELEKRVIKSEEIQKLIND
GCCCAPNFITDTYGIPEKRQKFEEYSEDMDTKIETIYLKTDTPLEYVEKVFEASQSENSFPLFQFDQSKK
YEWLANLTFVKRFIQCGGEIFRAENLEFRDFTLCGIITVLDTSTDILIDSPHSFSENPLLEALTTLYLEL
FVVLTDATKRGAYSEQSVEEWNEFYTPTIHTYCIRLFRTIRRDQQPTPFVPALLPALFVISEFPTSFSND
DDVANQEFIPELSVFKYPAFQESCIAQAFSLFASNNEHIQLIAYSVARLLMPIMFKLENAAALKSNEDSE
LPVSTNRRKLSLPVMISKSYPKDHHNPHVGPLLLDLTLLPLENTKDSGFSQEHRVAYCDVIDPFFKNALN
ALMLDQPFEFRQVPIGCRIQNRKFERKLKKNVKKSEILQFFCRKYGTPGLDAINLCLKKLRTSFYILLKF
GFVEISTIFRNMDRKKAEIFKNRKTNNFRSIFHQILFMLMRKKPIKKPRTKGNKCLCFFLININKTRFKN
FDFLKKRKIENSKSQEPAYYLESDLSASPIFFDKFASRLLFKSMTLLPAAIRLFYKGMPNCFMPMFQETV
TKYASRLLIEQELGKVREAKFEGEMKVRTVPVTGEIIAEYVVEETKMKLTIGLPPDYPLSVPSLTLDKAI
VKTDRAKKWLLQLNAYLFHQNGAILEGIEMWKRNVDKGVEGVEDCTICMMTVHQQTHQLPKIKCKQCKNK
FHSNCLYKWFESSNQSTCPLCRNNFT

Wild type Saccharomyces cerevisiae Sensin polypeptide has the following sequence (SEQ ID NO: 11). N-terminal lysine-rich domains are underlined, and RING domain homology is italicized:

MSFGGINTFQQYNTDLGLGHNGVRISLNYFDGLPDPSLLNSLYSNELKLIFKSLLKRDETTKEKALMDLS
NLISDFNQNEYFFNDIFLLCWSQIYAKLIISDYKVIRLQSHQITIMLVKSLRKKISKFLKDFIPLILLGT
CELDYSVSKPSLNELTECFNKDPAKINALWAVFQEQLLNLVKEIVVNENEDTISDERYSSKEESEFRYHR
VIASAVLLLIKLFVHNKDVSERNSSSLKVILSDESIWKLLNLKNGQNTNAYETVLRLIDVLYTRGYMPSH
KNIMKLAVKKLLKSLTHITSKNILKVCPVLPSILNLLATLDDYEDGTIWSYDKSSKEKVLKFLSVSRTSP
SPGFFNAVFALYSSTKRHSFLDYYLEWLPFWQKSVQRLNEKGFSARNSAEVLNEFWTNFLKFAEDSSEER
VKKMVESEIFNSLSCGKSLSEYTKLNQTLSGVFPPDKWEREIEDYFTSDEDIRKIKVSFEKNLFALLVTS
PNNESAISRLFDFFVQLIETDPSNVFNKYDGVYDALNYFLDSDMIFLNGKIGKFINEIPTLVQESTYQNF
AGIMAQYSNSKFFKMNTDAITSLEDFFIVALSFNLPKTIILATMNELDNDIYQQLMKSDSLELELYIEDF
MKNYKFDDSGEIFKGNNKFLNQRTITTLYRSAVANGQVEQFCAVLSKLDETFFSTLLLNTDFLSCALYEV
SEDTNEKLFKLSLQLAKGNSEIANKLAQVILQHAQVYFSPGAKEKYVTHAVELINGCNDTSQIFFPANAI
EVFARYMPAIDYRSSLVSSLSTNTHLLLTDDKPINLKNMQKLIRYALFLDALLDALPERVNNHIVAFITV
VSELVTDYNCLSEEPNDLYYDFGHTFFKHGKVNLNFSDIVGNVIQPANGGDAMLTFDIAESNSVYFFYYS
RVLYKVLLNSIDTVSSTTLNGLLASVESFVTKTVRDQKSTDKDYLLCAILLLMFNRSNSKDEITKLRTLL
ASQLIGIREVELVDQEFKSLALLNNLLDIPQADKQFVPIAPQRLNMIFRSILKWLDSDLAYEPSFSTVRL
LLLDFFTKLMRFEGVRDMGITAFELSERLLADSLSMCQIDDTLYLLELRSSCLNLYETLSQGVSKNGEEI
SEYGDEIQENLIELMFLNFNQERNNQVSTLFYQKLYKVISSMELKKLESQYKRIFEVVLNDKDIGSNINQ
SRLLTTLLGSLVVKTQQDIIIEYELRIQKQTGSDVDGSASDNDVNSKFKLPQKLLQKVTDEVPKEYLEYE
NKNSFIKYLWYWHLILMYFKDTSYNMRQIFIEQLKEAGLINRMFDFITDQIDLRDTEFWKQVDTKEISEY
NIVGNNFSPYKEDIFEECKKLLGHTLYQLFNNVGCLTSIWWLNIKDRTLQNDIEKFVSEFISPILIKNEF
DDINSKMDRLTSNDDALTIKLNNITNEVKASYLIDDQKLEISFKLPKNYPLTNIQVNGVSRVGISEQKWK
QWIMSTQHVITGMNGSVLDSLELFTKNVHLQFSGFEECAICYSILHAVDRKLPSKTCPTCKNKFHGACLY
KWFRSSGNNTCPLCRSEIPFRR

Wild type mouse Sensin genomic DNA fragments have the following sequence (50 basepairs of intronic flanking sequence is in italics on either side of exon):

(SEQ ID NO: 12) exon 1:
GAATCCCCTTCCGGCGCGAGGGACCGGAAGTTGTGGCCCGCCCGTGTCAACTTCGGCTCTGCAGACTGAGAGCCGGG
CTGCCACCGCCACCATGGGCGGGAAGAACAAGCAGCGGACTAAGGGGAACCTCAGGGTGAGTCCTGTGGTGATGGTA
GCCCCGCGGCCTGCGGCCCGGGTGTCCAG
(SEQ ID NO: 13) exon 2:
AGAAAGTGTTCTAACTTTTTTGTTTTTGCTTTGTTTTGCTTTCTTATCAGCCTTCAAACAGTGGCCGAGCTGCAGAA
CTCCTCGCCAAAGAGCAGGGAACAGTGCCTGGGTTCATTGGTTTTGGCACATCTCACAGTGACCTGGGCTATGTTCC
GGCTGTTCAAGGCGCGGAAGACATAGACAGTCTTGTAGATTCCGATTTCCGAATGGTGCTGCGGAAACTTTCCAAAA
AAGATGTTACAACAAAGCTAAAGGCAAGTTTTCTCATTTTTACAATAGTAATTAAGAATGTAGTTTTGTTAGAATAA
(SEQ ID NO: 14) exon 3:
GGTCGTGTTACCTCTGTTAACCCTTGATGATGTTAATTATTTTCAGGCAATGCAAGAATTTGGAATTATGTGCACAG
AGAGAGACACAGAAGCCGTCAAAGGGGTTCTTCCATACTGGCCAAGAATCTTCTGCAAAATCTCCCTTGTAAGCAAG
CATTAGCGTTCATGAGCTGTTTCTGCTTAAAGGGCTACCTC
(SEQ ID NO: 15) exon 4:
TCTTACAAATATTAGTATTTGTTCTTAAACCACATTTCTGCTTTCAATAGGATCATGATCGCCGTGTTCGAGAGGCG
ACGCAGCAAGCTTTTGAAAAACTTATCCTTAAAGTAAAGAAGCACTTAGCTCCCTATTTAAAAAGCGTGATGGGCTA
TTGGTTGATGGCTCAGTGTGACACATATCCACCAGCTGCACTGGCAGCAAAAGATGCATTCGAAGCCGCTTTCCCTC
CAAGCAAGCAACCTGAAGCCATAGCGTTTTGCAAGGAAGAAATTACAACTGTGAGTGTTTGCAGCCACTCTCTCCCC
GTGTTTCTCAGTTGTCTAAGGCT
(SEQ ID NO: 16) exon 5:
ATGGTTGTCAGTGCCATAAGTCTCAAGGCTTTGGTGGTTCTCTCCTTCAGGTGTTACAGGACCATCTTCTGAAGGAG
ACTCCTGACACACTCAGTGACCCTCAGTAAGCTGCATGTTTCTAACTAATGTGCACTAGGCATTTACTTAGTTGGT
(SEQ ID NO: 17) exon 6:
TGTAGTTTGGCTTCTTTTTTGCAGTCTTACTTGTTTATTTTTAACTCTAGAACTGTGCCAGAGGAAGAGAGAGAGGC
TAAGTTCCACCGAGTCGTGACGTGCTCTTTATTGGCACTGAAGAGATTGCTTTGTTTCTTACCTAACAATGAGCTTG
ATTCTCTGGAGGAGAAATTTAAATCACTTTTATCACAGAATAAATTTTGGAAGTATGGAAAACACAGTGTACCTCAG
GTATGCCAATAGCTTCGAGTTTAAAACTTGTTTTCTGTAACTCCGCCACG
(SEQ ID NO: 18) exon 7:
CTACTGTCTTTAGGCTTTGGTTTCATCCCGCTTTCTGGTTCTTCTTTTAGGTCCGCTCAGCATATTTTGAGTTAGTT
TCTGCTTTGTGCCAGCACGTTCCCCAGGTGATGAAAGAGGAAGCTGCCAAAGTGAGTCCATCTGTCCTGCTCAGCAT
TGATGACAGTGACCCTGTGGTCTGCCCAGCTCTCTGGGAGGCTGTGCTCTACACGCTGACAACTATTGAGGTATGTA
AGGGAGACACACTTCCCTAAACGCCACGTGAAAGAGTGCATTA
(SEQ ID NO: 19) exon 8:
CACTTACAATTTTTAATTACTATTATAAATTTTTTTTCTCTTTTGGTTAGGACTGTTGGTTTCATGTAAATGCCAAG
AAAAGTGTGTTTCCGAAGCTGATGGCCATGATCCGAGAGGGTGGCCGGGGCCTAGCTGCTGTTATGTATCCTTACCT
CTTACCGTTCATCAGCAAACTCCCTCAGTCCATCACAGAGCCAAAGCTGGACTTCTTCAAAAACTTCCTCACCTCTC
TAGTTACCGGGTGAGCAAATACATTTCTTTTTACTTTAAAAATTACGTTTATTGTTTATT
(SEQ ID NO: 20) exon 9:
GAGCTCTTCTTTTCCTCTTGGAAGAATAACCCTCAGTCACTACTTCCTAGGCTGTCAACTGAGAGGACCAAATCGAG
CTCTTCTGAGTGCTCAGCTGTCATCCCGGCATTTTTTGAATGTTTGCGGTTTATAATGCAGCAGAACTTAGGCGAGG
AGGAGATGGTGCAGATGCTTATCAATGAGCAGGTACGTTTGCTCAGCATCCTTATCAATGAGCAGGTACGTTTGCTC
AGCAT
(SEQ ID NO: 21) exon 10:
TTAAACTCTTCTTGAAGACTAAAATTTAAATTTTGTGCCTTCTCTTTCAGTTGATCCCATTTATTGATACAGTCCTC
AAAGACTCAGGACTGCACCACGGACCGATGTTTGACCATTTAGCAGACACATTGAGCTCTTGGGAAGCCAAAGCAGA
TGCAGAGCGAGACCCTGGAGCTGTTTACAACTTGGAGAACGTTCTGCTAAGCTTCTGGGGAAGACTGTCAGAGATCT
GCACCGAGAAAATCCGCCAGCCAGAAGCAGATGTGAAGTCCGTTCTGTGTGTGTCTAGCCTAGTAGGGGTCCTTCAG
AGGCCAAGGAGCTCACTGGAGTTACACAGAAAGAAAACTGCTCAGGTCAGGTTTGCCATCAATATACCTGAAGCTCA
TAAAGGGGATGAAAAGTCCATGTCTTCAGAAGGAGAGAACAGCGAGGGCTCTGACGGAGGGGCTCAGTCTCCTCTCA
GTAATACATCTTCAGACTTAGTATCTCCTTTAAGGAAAAAACCTCTGGAAGACTTAGTCTGTAAGCTGGCAGAGGTG
AGCATTAGCTTTGTCAACGAGCGGAAGTCAGAGCAGCACCTGCAGTTTCTCTCCACGCTGCTCGACTCCTTCTCCTC
CGTCCAAGTATTTAACATTCTCCTCAGTGACAAACAGAAGAATGTCGTCAAAGCCAAACCTCTGGAAATAACCAAAC
TTGCAGAAAAAAATCCTGCAGTTAAGTTCTTATATCATAAATTGATAGGCTGGCTAAATGACAGCCAGAAAGAGGAC
GGGGGCTTCCTGGTGGACATCTTGTATAGTGCCCTCCGATGCTGTGACAGTGGTGTGGAAAGGAAAGAAGTCTTGGA
TGATCTAACCAAGGTACGGCTGTCATCTGACTGTCATAAACATCCTTGTTGTGTGTGGTAAGG
(SEQ ID NO: 22) exon 11:
TTTTTAAAGCTGTATTTTTGTCCTTAACTCTCCAACTTTGTCTGTTTCAGGAGGACCTGAAGTGGAGTTCTCTTCTT
CAGGTCATTGAAAAGGTATTTTAGCCCCCTCTCCCCTTTCCCTTTCTTTTTCAATTTATGTGTGT
(SEQ ID NO: 23) exon 12:
TAGACACATCTTTTGATGTTGCTTTTAATCTTTAATTTTATGGTTTTTAGGCATGTTCTAGCTCAGATAAACATGCT
TTAGTAACTCCTTGGCTAAAAGGCAGTATTCTTGGAGAGAAATTGGTTGCCTTGGCAGACTGTCTTTGTGATAAGGA
CTTGGAAGCCACAACATCTGAATCCCACTCATCAGAACAGTGGAGTCTGCTAAGACTGGCGTTATCCCAACATGTCA
AAAACGGTAGGGAATACAACTCCAGCTTTATAGCAGGGTTGATAATCCTTTCCACC
(SEQ ID NO: 24) exon 13:
AAGATATTTTCAAAATACATTGTTAATACTCTTTACATTTTTTTTTGTAGATTACTTGATTGGAGAAGTATACGTTG
GAAGAATTATTGTTAAACTTCATGAAACTTTATCTAAAACAAAGGATTTATCAGAAGCGGCAAACAGTGACTCTTCA
GTGTCTTTTGTCTGTGATGTGGTCCATAGCTTCTTCAGTTCAGCAGGAGGAGGCTTGCTAATGCCACCATCTGAAGA
CTTGTTATTAACTCTCTTTCAGTTATGCGCTCAGAGCAAAGAACGGACACACTTGCCAGGTAAGAGCCTGCTGCTCA
GATATTTTCTAGGCAATCTCGGCTCTGACCTC
(SEQ ID NO: 25) exon 14:
AGTCTATTTATAGTGAGATTACAAATTATCGCTAATACCTTGATTTTTAGATTTTCTGATCTGTAAACTGAAGAATA
CTTTGCTTTCTGGTGTAAATTTATTGGTCCATCAAACTGCAAGTACATATGAGCAGAGTACCTTCCTACGTTTGTCT
GTTCTGTGGCTGAAGGACCAGGTTCAGTCTTCGGCTTTGGATAACACAAGGTAACATTTTGTGCACAGTTACATGGT
GTCCCTTATATACTGTCTCCTTC
(SEQ ID NO: 26) exon 15:
TATGGAATTGCTTGTACCCTAACTACAGCGCTTCTTTCCATTGTTTTTAGTCTTCAGGTCCTCTTGTCTGCTGCTGG
CGACTTGCTGGGTACTCTTGTAGAGAGTGAGGACACGTCTCTTCTTGGAGTTTATATCGGAAGTGTAATGCCCAGTG
ACAGCGAGTGGGAAAAGATGAGGCAGGCTCTTCCTGTTCAGGTGTGTCTTCCACTGCAGCGCGCAGCTCATCCTGGA
TTTCTAGTCCATTC
(SEQ ID NO: 27) exon 16:
TTTAGACTATCGAATGAATTTTCATACAATTGAGTGTGTTCTATTTACAGTGGTTACACAGACCTCTTTTAGAGGGA
AGGCTGAGTTTAAATTATGAATGCTTCAAGACAGATTTTAAGGAACAAGACACAAAGACTCTTCCCAACCATCTGTG
TACTTCATCGTTATTGAGCAAAATGATCTTAGTTGCCCAAAAAAAGAAATTAGTCCTAGAAGACAACGTCCTTGAAA
AAATAAGTAAGTATCTGCGAGGTTCAGGTAGACACTCTGTGATCTTCTTCTGTGCT
(SEQ ID NO: 28) exon 17:
GGTTCTGTACAGTGTGTGCCTTTGATAACTGGCTGTGTCCATTCTCTTAGTTGCAGAGCTGCTGTATTCACTGCAGT
GGTGTGAAGAACTCGACAATGCGCCATCTTTCCTAAGTGGATTTTGTGGAATCCTTCAAAAAATGAATATAACTTAC
AGCAACTTATCTGTACTTAGTGAGACTTCTAGCCTCTTGCAGCTGTTATTTGACAGGTAAGACGCCCTTTCAATTTG
TTTTAAAATTGGCTTTCCCATCCATAAGA
(SEQ ID NO: 29) exon 18:
CTCTATCTGTATAGCTATATTTAACATTCATGTTTTGCTTCTTTTTTTAGATCTAGAAAAAATGGCACACTTTGGTC
TCTTATTATTGCTAAGTTGATCCTTTCCCGAAGTATTTCATCTGATGAAGTGAAACCATACTATAAAAGAAAAGAAA
GGTACCCTTAGTTACTATGTTTCCACTCTGCAGTTTATTATTAGAGTGGGT
(SEQ ID NO: 30) exon 19:
CATCATTTTGCACATAGAATAACTGTAAGTAATGTTTTCTCTCTTAACAGTTTCTTCCCTCTAACAGAAGGTAGTTT
GCATACCATTCAAAGTCTCTGTCCATTCTTGTCTAAAGAAGAGAAGAAGGAATTTAGTGCTCACAGTATACCCGCTT
TTTTGGGTTGGACTAAAGAAGACCTTTGCAGTATTAATGGTAAGTCACTGGGCAACATTCTACATTTTAATTGTTTC
TTGAGATTAGAG
(SEQ ID NO: 31) exon 20:
TTAGACGGGGTTTAAAATGGACTTTCCTTTTCGCTCTGATTCTCCTTTAGGAGCCTTTGGACATCTTGCCATTTTCA
ATTCTTGTCTGCAAACCAGAAGTATAGACGACAAACAGCTATTGCATGGCATATTAAAAATTATAACAAGCTGGAGG
AAACAGCATGAAGATATTTTTCTTTTTAGCTGGTAGGTAGTACTTTATTCCCACATTAAAATGTGGGCCCAGATTCT
GTATT
(SEQ ID NO: 32) exon 21:
TGGTTTAAAATGGAATATTCTCTAATTGAATTTCTTTGCCTTTATCACAGTAATCTGTCGGAAGCGAGTCCAGAGGT
CCTTGGTTTAAACATAGAGATCATGCGGTTCCTTTCCTTGTTTCTCAAGCACTGCGCGTACCCGCTCCCGCTGGCAG
ACAGTGAATGGGACTTCATCATGTGCTCCATGCTGGCTTGGTTGGAGGTAAATCAGCCCGATGTGTCATCCCCTGAG
TTGGGCCTTTGCACACTCTG
(SEQ ID NO: 33) exon 22:
GGTGTTGGGTAGCACCTTGCTTACCTGTTTTATTTCTGTCCTTTCCTTAGACAACCAGTGAGAACCAGGCTCTGTAC
TCTGTTCCACTTGTGCAGCTGTTTGCCTGTGTCAGCTTTGATCTGGCCTGTGATCTCTGTGCCTTTTTTGACTCAAT
AACTCCAGATATTGTTGACAATCTTCCTGTAAATCTCATCAGTGAGTGGAAAGAGTTTTTTTCTAAAGGCATCCACA
GTTTGCTATTACCTCTTTTGGTAAATGCTATCGGTAAGTGCAAAGGGGTATGATGATGGTAATTATTGAAAATCGCT
GTTGAC
(SEQ ID NO: 34) exon 23:
ATGTGTGTTCTTATGAGTTGTGTGTGTGTGTTTCTTCATTTCAATACCAGGAGAAAACAAAGACCTATCTGAAACGT
CCTTTCAGAACGCAATGCTGAAACCCATGTGTGAAACACTAACATACATCTCCAAGGACCAGCTACTGAGCCACAAG
CTCCCTGCGAGATTGGTTGCCAGCCAGAAAACAAACTTGCCAGAGCACCTCCAGACTCTGCTGAACACTTTGACCCC
ACTGCTTCTCTTCAGAGCCAGACCTGTGCAAATTGCTGCTTATCATATGCTGTGCAAGTAAGACATTGGCAGTGGCA
TAAGTATTTGGTCTCTAAAGCATGATGCAT
(SEQ ID NO: 35) exon 24:
TAGTAGACAGTTGGCTATAAATGTGTTAAAAGTGATGTTTCGCTTTTCAGACTGATGCCTGAATTGCCACAGCATGA
TCAGGACAATCTGAGGTCGTATGGAGATGAAGAGGAAGAACCAGCCTTGTAAGGGTTTTGAGTGTGTGTTTGCTTGT
GTGAGTGCAGGTTACAGGACC
(SEQ ID NO: 36) exon 25:
AGATGTTTGTTCGAGACAGGTGAATGGTCCTGTGTGACGTTGTTTCCCAGGTCGCCGCCGGCCGCGCTGATGTCTCT
CCTCAGCTCTCAGGAGGAGCTGCTGGAGAATGTCCTGGGCTGTGTCCCTGTGGGCCAGATCGTGACCGTTAAGCCAC
TGAGCGAGGACTTCTGCTATGTCCTGGGATACCTCCTCACTTGGAAGTTAATACTGACTTTCTTCAAAGCTGCATCG
TCTCAGGTAAATAAGTTCTGGGCTGTTGGAGGGTGGGGTGCTGTTGCATTGCAGTG
(SEQ ID NO: 37) exon 26:
TTTGTCTTGGCACAGTTGAGTTTACTAACACATTCTTTGCTTTTCACCAGCTTCGTGCTCTGTATTCAATGTACCTT
CGGAAAACAAAGAGTCTGAATAAATTACTCTATCATCTCTTCAGACTTATGCCAGAAAACCCTACGTACGGAGAGAC
AGCTATTGAGGTATCAAGTAAAGACCCCAAGACCTTCTTCACCGAGGAGGTTCAGCTGAGTATTAGAGGTCAGTGGG
CTACACGTGTGTGGGCTGCACATGTGTGGGCTACACGTCTG
(SEQ ID NO: 38) exon 27:
TCTAGGTGAGTGCACATGTATCACTAACCTGGGATTTGTTTCTTCCTCAGAAACAGCAACTCTTCCGTATCATATCC
CACACCTGGCGTGCTCGGTCTATCACATGACTTTAAAAGACTTGCCTGCCATGGTTAGGCTATGGTGGAATAGCAGT
GAGAAGCGTGTCTTCAATATTGTAGATAGATTTACAAGCAAGTATGTCAGCAATGTTCTTTCTTTTCAAGAAATATC
TTCTGTACAAACAAGTACACAGCTATTCAATGGCATGACGGTTAGTATTGTCTTGGTTTTTTTCTAGAGAAATGAAT
ACCAGTTATATTT
(SEQ ID NO: 39) exon 28:
AAGGAAAAATACCTTTTTTTTGTATTGTATTGTATTGCATTGTATTGTAGGTTAAGGCGCGAGCTACTACTCGAGAA
GTGATGGCTACGTACACCATCGAAGACATAGTCATTGAACTCATAATACAGTTGCCTTCCAATTACCCACTGGGCTC
AATAACAGTGGAAAGTGGGAAGAGGATCGGGGTGGCTGTGCAGCAGTGGCGAAACTGGATGCTGCAGCTGAGCACGT
ACCTCACTCACCAGGTATGCTGCCACGAGCGCACTGGTCACTCACACTAGAGCACATCTGTCTG
(SEQ ID NO: 40) exon 29:
CATTTGGAGACCTTTAGTGTGTGGTGACTTTCTTTCCTCTCTACTAACAGAACGGAAGTATCATGGAAGGCTTAGCA
TTATGGAAGAATAATGTAGACAAACGGTTTGAAGGTGTTGAAGATTGTATGATCTGCTTCTCAGTTATTCATGGTTT
CAACTATTCTCTTCCCAAAAAAGCCTGTAGAACATGCAAGAAAAAGTTTCACTCAGCTTGCCTGGTAAGGCGAAGGG
AAATCTCTTAACATTCTTTGTGGTCTGTATATGTTAT
(SEQ ID NO: 41) exon 30:
ACGAAAGCCTTACAAATTTACTTTAACATTAAAACCTTTTTTTTTTTCAGTACAAATGGTTTACATCTAGCAACAAG
TCCACTTGCCCGCTCTGCCGTGAGACCTTTTTCTGAGGTTTTTTTCATTGGAAGTTGTCGCTGCCGTAGGTCAAGCC
AAAGGGAATGGATTGGCTCCACCTTGAAGTACTGATGTGAAGCCAGTGAGCATGACAAAGTGCCATCTGTCAGTATG
ATCCTCAAATCAGGCAACCTCTGCAGATTGCTTTGTAAAT
A preferred wild type mouse Sensin genomic DNA subfragment has the following
sequence (SEQ ID NO: 42):
CTTCTTCAGGTCATTGAAAAGGTATTTTAGCCCCCTCTCCCC
A preferred mutated mouse Sensin genomic DNA subfragment has the following
sequence (SEQ ID NO: 43) (the mutation site, relative to mouse wild-type
Sensin is in bold underline):
CTTCTTCAGGTCATTGAAAAGGAATTTTAGCCCCCTCTCCCC
Wild type human Sensin genomic DNA fragments have the following sequence
(50 basepairs of intronic flanking sequence is in italics on either side of
exon):
(SEQ ID NO: 44) exon 1:
CCGGTGGCGGATGGTCAGGGACACAGCGGGAGGCGGGAGCGGAAGTGGGGCAGCAAATGGACAGGGTGGGTGGCGGAA
AAGGGCCCGGGGGAAGTTATTACAGGGTGTCCTCTTCCGCCGCCAGAAGCCGGAAGTTGTGTCCCGGACGTGTCAACC
GGGGTCTGAGTGCTCAGAGTACAGCTGCAACCGCGACCATGGGCGGGAAGAACAAGCAGCGAACTAAAGGGAACCTGA
GGGTGAGCGGGGGCTGGCTCGGCCGACCCGGGAGCCTCGCTTCGAGGCGGCT
(SEQ ID NO: 45) exon 2:
TTTGAACCCTTCCTGGACTCTGACTTTTTTCTCCGTAATTTTCTTATCAGCCTTCAAACAGTGGCCGAGCTGCAGAA
CTCCTTGCCAAAGAACAGGGAACAGTGCCTGGATTTATTGGTTTTGGAACATCTCAGAGTGACCTAGGCTATGTTCC
TGCTATTCAAGGAGCTGAAGAAATTGACAGTCTTGTAGATTCTGATTTCCGAATGGTGCTGCGGAAACTTTCAAAGA
AAGATGTCACCACAAAATTAAAAGCAAGTTTTCTTGTTTTCATAAAAATTATCAAGAAAATCCCTTTGTTAAA
(SEQ ID NO: 46) exon 3:
GCCAGTTATTATGGTTGTTAATCCTAATGATGTTAATAACTTACTTTTAGGCTATGCAGGAATTTGGAACCATGTGT
ACAGAGAGAGACACAGAAACTGTGAAAGGAGTTCTTCCATATTGGCCAAGAATTTTTTGCAAAATTTCACTTGTAAG
TATTAAAACTTTGCTAGTTTATTTCTGTTGTATATTTTTTGGTTG
(SEQ ID NO: 47) exon 4:
TTGTTTTTATGGGCTAGAGCTATTTTAAATCACATTTATATTTTTAATAGGATCATGACCGTCGCGTCCGAGAAGCC
ACACAACAAGCTTTTGAAAAACTTATCCTTAAAGTAAAGAAACAGTTGGCTCCCTACTTAAAAAGTTTAATGGGATA
TTGGCTAATGGCTCAGTGTGATACTTACACACCAGCTGCGTTTGCAGCAAAAGATGCATTTGAAGCGGCTTTTCCTC
CAAGCAAGCAACCTGAAGCCATAGCATTTTGTAAGGATGAAATTACAAGTGTAAGTTCTGGAATCATTCTGAATCTA
TTTTTTTTTTTTAAGTATTTAAG
(SEQ ID NO: 48) exon 5:
CATTGACAGTTCAGTAAAACCAAAGGCTTTGGTATTTCCCCCCTTTGTAGGTGCTGCAGGATCATCTTATAAAAGAA
ACACCTGATACACTCAGTGACCCGCAGTAAGTTGTATTGTTTCATTGTAACTCATGTTAAGGATTTGTTTCACTCA
(SEQ ID NO: 49) exon 6:
GTTTGGCTAATTCTAAATTTTTAAATATATTTATTTTGTTTTATATCAAGAACTGTTCCAGAGGAAGAAAGAGAAGC
TAAATTCTACCGGGTTGTAACTTGTTCCTTATTGGCATTAAAGAGATTACTTTGCCTTTTACCTGATAATGAGCTTG
ATTCTCTGGAGGAGAAATTTAAGTCTCTTTTATCACAGAATAAGTTTTGGAAGTATGGAAAACACAGTGTACCTCAG
GTATATTAATCTTTTTTATCTTAAGACATTTCTCTGATTCCTTACCCCCA
(SEQ ID NO: 50) exon 7:
TTATCAAGTTTTACTAGGTTTTAATTTAATTCAGCTTTGCATTCTTTTAGATTCGCTCAGCTTATTTTGAGTTAGTC
TCTGCATTGTGCCAGCGCATTCCACAGTTGATGAAAGAGGAAGCATCCAAAGTGAGCCCATCAGTTCTACTTAGCAT
TGATGACAGTGACCCAATTGTCTGCCCAGCTCTCTGGGAAGCTGTACTCTATACACTTACAACTATTGAGGTATGTA
AGAGAGGCACATTTAGTACACTGAGGAATGAACCTATGAGATA
(SEQ ID NO: 51) exon 8:
AAAGTATGTTTATGATCTCTAATTACTACTATAATCTTTTGTTTGGTTAGGACTGTTGGCTTCATGTAAATGCAAAA
AAGAGTGTGTTTCCCAAGCTATCAACTGTGATTCGTGAAGGTGGTCGGGGTCTAGCTACTGTCATATATCCTTACCT
TCTGCCATTCATCAGCAAGCTCCCTCAGTCCATCACAAATCCAAAGTTGGATTTCTTCAAAAATTTCCTCACGTCTC
TAGTTGCTGGGTAAGTAATTTAAATTTTTGATTTTTAAAACAAAACAGATTTTCTTGTTT
(SEQ ID NO: 52) exon 9:
TCTTGTTTCTTCTTCATGAAAGAGTAATCCAGTTATTAATTATTTCTTAGGCTGTCAACAGAGAGAACTAAAACCAG
CTCTTTAGAGTCCTCGGCAGTAATATCTGCTTTTTTTGAATGCTTACGTTTTATAATGCAGCAAAACTTAGGTGAGG
AAGAGATTGAACAGATGCTCGTCAATGATCAGGTATCTATAATGTAAAAGTCGTCAGTCTCTTTGCATACTGATTAT
GTAGG
(SEQ ID NO: 53) exon 10:
GGGGTTGAAGACCAAATATATATTTTATGATATTCTACCTTTTCTTTCAGTTGATCCCTTTTATTGATGCAGTTCTC
AAAGACCCAGGATTGCAACATGGGCAGCTATTTAACCATTTAGCAGAAACTCTAAGTTCCTGGGAAGCCAAAGCAGA
CACGGAAAAAGATGAAAAAACAGCTCACAACTTGGAGAACGTACTGATACATTTCTGGGAAAGACTGTCAGAGATCT
GTGTTGCGAAAATCAGTGAGCCAGAAGCTGATGTTGAGTCCGTTTTGGGTGTATCTAACCTATTACAGGTGCTTCAG
AAGCCGAAGAGCTCATTGAAGTCAAGTAAAAAAAAAAATGGTAAGGTTAGATTTGCTGATGAGATACTTGAAAGCAA
TAAAGAGAATGAAAAATGTGTATCTTCAGAAGGAGAGAAGATTGAAGGCTGGGAATTAACAACTGAACCTTCTCTCA
CTCATAATTCTTCAGGCCTTTTGTCTCCTCTAAGGAAAAAACCTTTGGAAGACTTAGTCTGTAAACTCGCAGATATA
AGTATTAATTATGTCAATGAACGAAAGTCAGAGCAACATCTAAGGTTTCTTTCTACTCTGCTTGACTCCTTTTCTTC
AAGCCGAGTATTTAAAATGCTACTTGGTGATGAAAAACAGAGTATTGTCCAAGCCAAACCTCTTGAAATAGCCAAGC
TTGTACAAAAAAATCCTGCGGTGCAGTTTTTATACCAGAAACTGATAGGTTGGCTAAATGAAGATCAAAGGAAGGAT
TTTGGTTTCCTGGTGGACATTTTGTACAGTGCTCTCCGGTGCTGTGACAATGATATGGAAAGAAAAAAAGTCTTGGA
TGATCTAACCAAGGTATTCCTGTTGTATATCTTTTCAAACTATTTGAATAATAGATGAGTAGA
(SEQ ID NO: 54) exon 11:
AACAAAAAGTTGTTCTAGCAGATTGTTTCTAACTCTTTTTCTTTTTTCAGGTGGACTTGAAATGGAATTCTCTTCTT
AAGATTATTGAAAAGGTATCTTAGGGATTTTTTTTTCTTTTTTTTTTTGTATTTATGGGAATAGA
(SEQ ID NO: 55) exon 12:
GAGAGTATTTGATCTTGCTGTTAATCTTTGCTTTAATTTTATGGTTTTAGGCATGTCCTAGTTCAGATAAACATGCT
TTAGTAACTCCTTGGCTCAAAGGCGATATCCTTGGTGAGAAATTGGTCAACTTGGCAGATTGTCTTTGTAATGAGGA
CTTGGAATCCAGGGTATCTTCAGAATCTCACTTCTCAGAAAGATGGACTCTTCTAAGCTTGGTATTATCCCAACATG
TTAAAAATGGTAGGACAAATATGGCTTTTGTTTTCTAATGGGGAATAGTCCATTTCTCC
(SEQ ID NO: 56) exon 13:
TACACGTTAATATTTTTATTTTTTGTCTGTTTGAACCTTTTTTTCTCCAGATTACTTGATTGGAGACGTATATGTTG
AAAGAATCATTGTTAGACTTCATGAAACTTTATTCAAAACAAAGAAATTATCAGAAGCTGAAAGCAGTGACTCATCA
GTGTCTTTTATCTGTGATGTGGCCTATAACTATTTCAGCTCAGCGAAAGGATGCTTGCTAATGCCATCATCTGAAGA
TTTATTATTAACTCTCTTTCAGTTATGTGCTCAGAGCAAAGAAAAAACACATTTGCCAGGTAATAGCCTACTGCTCA
AATGTTTTGTTGGGAATCTCCGGCTCTGACCT
(SEQ ID NO: 57) exon 14:
GAGAGTTAATTCAGTGAGATTACAAATTCCTGTTGACATCTTGATTTTAGATTTTCTTATCTGTAAACTGAAAAATA
CTTGGCTCTCTGGTGTAAATTTATTGGTTCATCAAACTGACAGTTCATATAAAGAGAGTACCTTCCTACATTTGTCT
GCTCTGTGGCTGAAGAACCAAGTTCAGGCTTCATCTTTGGATATCAACAGGTAACCTTTTTTGAGCTTGGTTTCAGT
GTCTCTTTTATACTTGAACAGTG
(SEQ ID NO: 58) exon 15:
AACTAGTCATAGCGTAACTACAACAGTTAAGTTCTCTGCATTATTTTTAGTCTCCAAGTCCTCTTGTCTGCTGTTGA
TGATTTGCTAAATACACTTCTAGAGAGTGAAGATTCTTATCTTATGGGAGTTTATATTGGAAGTGTAATGCCGAACG
ACAGTGAATGGGAAAAGATGAGGCAGTCTCTTCCTATGCAGGTATTTTGGAAATTGAAGAGTACATATCTCATTCTG
AAGTTTGGATTTCA
(SEQ ID NO: 59) exon 16:
CAATTGCTTAGGTAATAAATAATTTTGTATATAACGTATTCTTTTCACAGTGGTTACATAGACCTCTTTTAGAGGGA
AGATTGAGTTTGAATTATGAATGTTTCAAAACAGATTTTAAGGAACAGGACATAAAGACACTTCCCAGCCATTTGTG
TACTTCAGCATTATTGAGCAAAATGGTCTTAATTGCACTGAGAAAGGAAACAGTCTTAGAAAATAATGAGCTTGAGA
AAATAAGTAAGTATATATGAGTATTTACATATAACATAATGCATCAATGAATATAA
(SEQ ID NO: 60) exon 17:
GGTTTGTTCACAGAGTGCTTTTCATAATTTGGTGCTGTCTCTTCTTTTAGTTGCAGAACTGCTTTATTCACTGCAGT
GGTGTGAAGAATTAGATAACCCACCTATTTTTCTAATTGGATTTTGTGAAATACTTCAAAAAATGAATATTACGTAT
GATAACTTACGTGTACTTGGTAATACGTCGGGCCTTTTGCAGCTGTTATTTAACAGGTAAGAATCTCTTTCAATTTG
TTTTTAAAATGACTATGCTACTTCTTTAT
(SEQ ID NO: 61) exon 18:
ATTCAGTATGCTTTCAAAACGGAATATAATTTTTTTCTTTCCTTTTTCAGGTCCAGAGAACATGGCACACTGTGGTC
TCTTATTATTGCTAAGTTGATCCTTTCCCGAAGCATTTCATCTGATGAAGTAAAACCACATTATAAGAGAAAAGAAA
GGTATTCTTATTTAAAATGTTTTTACTTTGTAGTTTACTGTAATCGGTCAC
(SEQ ID NO: 62) exon 19:
ACTGTATGTTTGGAAGAAATCTAACTTGTGTTTTTTTTTTCTTTAAACAGTTTTTTTCCACTAACTGAAGGCAATTT
GCATACCATTCAAAGTCTATGTCCATTTTTGTCAAAAGAAGAAAAGAAAGAATTTAGTGCTCAATGTATACCTGCTC
TTTTGGGCTGGACTAAGAAAGATCTTTGCAGCACTAATGGTTAGTTGCTTGATAATAAATATTTCATTAATTTAATT
ATTGCTTGAGAT
(SEQ ID NO: 63) exon 20:
GCGATATAGTTCTTAAATATTTTTAATTTTCATTTTGAATTTTACTTTAGGAGGTTTTGGACATCTTGCCATTTTCA
ATTCTTGTCTGCAAACCAAAAGTATAGATGATGGAGAGCTATTACATGGAATATTAAAAATCATAATATCCTGGAAG
AAAGAGCATGAAGATATTTTTCTTTTCAGTTGGTAGGTGATACTTTATACTATGTTTTTCCTTCAGTATACAATTCA
GACAA
(SEQ ID NO: 64) exon 21:
TGAATGTCAATATAATCTTTTATTGTCTTTTGTTTTTATCTTTATCACAGTAATCTATCAGAAGCAAGTCCAGAGGT
ACTGGGTGTAAATATAGAAATAATCCGGTTTCTTTCCCTATTTCTGAAATACTGCTCATCCCCTTTGGCAGAGAGTG
AGTGGGACTTCATCATGTGCTCCATGTTGGCTTGGTTGGAGGTAAATTAACCCGATATGTCATCACCTCTTGTGGGT
TTTTGTACATACTT
(SEQ ID NO: 65) exon 22:
GTGTTCTGGTAGAAGTCTGTTTACCTATTTCATTTTTATTGTTTTTTCAGACAACAAGTGAGAATCAGGCATTGTAT
TCTATTCCACTTGTGCAACTGTTTGCCTGTGTCAGCTGTGATTTGGCCTGTGACCTCAGTGCTTTCTTTGATTCCAC
AACTCTGGATACCATTGGCAATCTTCCTGTAAATCTAATCAGTGAATGGAAAGAATTTTTTTCCCAAGGCATCCACA
GTTTGCTTTTACCTATTTTGGTGACTGTTACAGGCAAGTGAAAAAGGGAATAATAGTGAGATTCATTCATTGGAAAT
GACTTA
(SEQ ID NO: 66) exon 23:
ATACTAACTTTCTTTTGAGACGTGCATGTTTCTTTCCTTTTTGTCATTAGGAGAAAACAAAGATGTGTCTGAAACAT
CCTTTCAGAATGCAATGCTGAAACCCATGTGTGAAACATTAACGTATATCTCAAAGGAACAGCTATTGAGTCACAAA
CTTCCTGCAAGATTAGTTGCTGACCAAAAAACAAACTTACCAGAATATCTCCAGACTTTGTTAAATACATTGGCCCC
ATTACTCCTCTTCAGAGCTAGGCCTGTGCAAATTGCTGTTTATCATATGCTATACAAGTAAGAATTCATCCAATTGA
ATCAATGTTACAGTGGTCTAAAAAAATAGA
(SEQ ID NO: 67) exon 24:
TAATAAACAGTTTAATATCACATATTTAAAAATAATGTTCTAATTTCTAGATTGATGCCTGAATTACCACAGTATGA
TCAGGATAATCTAAAGTCATACGGAGATGAAGAAGAAGAGCCAGCCTTGTAAGGTTTTTTTAAATAATTTGTTTTAT
TAAATTCTTATAATCCATCTC
(SEQ ID NO: 68) exon 25:
AAGGAATGAATTATAAAGAAAACTTGTGGTGTTTAATCATTGATTCTTAGGTCACCACCAGCAGCACTGATGTCTCT
TCTTAGCATTCAAGAGGACTTACTAGAAAATGTTTTGGGGTGTATTCCTGTTGGACAGATAGTTACTATTAAACCAC
TGAGTGAAGACTTCTGTTATGTTCTGGGATACCTTCTCACTTGGAAATTAATACTAACTTTCTTCAAAGCTGCATCA
TCACAGGTAAATAAATATGTGACAACTTTCGATAGTTCTGTCCTAATATGCTTCTG
(SEQ ID NO: 69) exon 26:
AATTGTCTTGGTGTATTTGGTTTACTAACAAATTCTTTGCTCTCCACCAGCTTCGGGCTTTGTATTCCATGTATCTT
CGGAAAACAAAGAGTTTGAATAAATTGCTCTATCACCTGTTCAGGCTTATGCCAGAAAATCCAACCTATGCAGAGAC
AGCAGTTGAGGTCCCAAATAAGGACCCTAAAACATTCTTTACTGAGGAGCTCCAGCTGAGTATTAGAGGTCAGTAAG
ATATGTGTTTATGTTCTTTCTTGGACACTAGATTCAGACTA
(SEQ ID NO: 70) exon 27:
TTCTATATAAGAAATATATGTTACTAATCAATAATTTGTTTTTTCCTCAGAAACAACAATGCTTCCATACCACATTC
CACACTTGGCTTGTTCAGTCTATCATATGACATTAAAAGACTTGCCTGCCATGGTTAGGTTGTGGTGGAATAGCAGT
GAGAAGCGTGTTTTCAATATTGTGGATAGATTTACAAGCAAGTATGTCAGCAGTGTTCTTTCTTTTCAAGAAATATC
TTCTGTACAAACAAGTACACAACTATTTAATGGCATGACGGTTAGTATTGTCTTGACTTTCTCTAGAAAAGTTGTTT
TAATATTGGGTAT
(SEQ ID NO: 71) exon 28:
GGTTGAAAGACAGAAATTTGGCAAACAAAATATCCTTTGTTGTATTGCAGGTTAAAGCTCGAGCTACTACTCGAGAG
GTAATGGCTACTTATACTATTGAGGACATAGTTATTGAACTTATAATACAACTGCCTTCAAATTATCCACTGGGTTC
AATAATAGTAGAAAGTGGGAAAAGAGTAGGAGTAGCTGTTCAGCAGTGGCGGAACTGGATGCTGCAGTTAAGCACTT
ACCTCACCCATCAGGTAAGTTTCTGTTTACACATTTGGCTTTACAAACTTGGAAAAGATGATCT
(SEQ ID NO: 72) exon 29:
AAATTGGTGGTTATCAGTGTGTAGTGACTTATTTTTCTTCTTGTTAACAGAATGGAAGTATTATGGAAGGCTTAGCT
TTATGGAAAAATAACGTAGACAAACGTTTTGAGGGTGTTGAAGATTGCATGATCTGTTTCTCAGTCATTCACGGTTT
CAACTATTCCCTTCCCAAAAAAGCCTGTAGAACATGCAAGAAAAAATTCCATTCAGCCTGCTTGGTAAGTCTAAAGA
GAAATTAACTTACTTATATTTTATGTATTTTATACAC
(SEQ ID NO: 73) exon 30:
GAGGAGAACTTTAAAAGTTCTTTTAGTATTTAAAACCTTTTCTTTTTCAGTACAAATGGTTTACATCTAGCAACAAA
TCCACTTGTCCACTGTGTCGTGAGACGTTTTTCTGAGATTTTTTTCACTGGAAGGGATCCCTGAAGTACATCAAACA
AAGGCATTG

A preferred wild type human Sensin genomic DNA subfragment has the following sequence (SEQ ID NO: 74):
CTTCTTAAGATTATTGAAAAGGTATCTTAGGGATTTTTTTTTCT

A preferred mutated human Sensin genomic DNA subfragment has the following sequence (SEQ ID NO: 75) (the mutation site, relative to mouse wild-type Sensin is in bold underline):
CTTCTTAAGATTATTGAAAAGGAATCTTAGGGATTTTTTTTTCT

Identification and Characterization of Novel Sensin Sequences

The Sensin gene was initially identified in the mouse genome on chromosome 16, band C3.3. A 5454 basepair mRNA sequence with a 5301 basepair coding sequence translates into a 1767 amino acid protein. The mouse gene is comprised of at least 30 exons spanning approximately 50 kilobases of sequence (FIG. 1). ESTs imply that the senseless gene is expressed during different developmental timepoints and in different tissues: we have documented its expression in the mouse brain and spinal cord by in situ hybridization (FIG. 3), and have detected expression in various other adult mouse tissues by RT-PCR, including heart, liver, and lung.

Identification of Sensin orthologs in other species can be performed using a number of methods known to those of skill in the art. These methods include computational genomic annotation, using programs such as BLAST and GeneScan (see, e.g., Lynn et al., J. Genet. 80: 9-16 (2001)); and biochemical methods, e.g., low stringency hybridization methods. For computational identification, BLAST version 2.0 is preferably used with parameters set at word size=3, expect=10, filter low complexity with SEG, cost to open gap=11, cost to extend gap=1, similarity matrix Blosum62, Dropoff (X) for blast extensions in bits=7, X dropoff value for gapped alignment (in bits)=15, final X dropoff value for gapped alignment=25.

In addition, one may isolate the genes encoding the novel polypeptides using methods known to the skilled artisan. For example, cDNA encoding a protein of interest may be identified by screening a cDNA library that can be obtained as an EcoRI-based lambda phage library (lambda ZAP) from Stratagene Cloning Systems (La Jolla, Calif., U.S.A.). The cDNA library may be screened, for example, using a plurality of random oligonucleotide probes constructed based on the known amino acid sequence obtained from a protein of interest using mass spectrometry. Exemplary conditions for screening comprise 6 times SSC, 25% formamide, 5% Dernhardt's solution, 0.5% SDS, 100 μg/ml denatured salmon sperm DNA, at 42° C. Exemplary processing of such screens comprise the following steps: filters are washed with 2 times SSC containing 0.5% SDS at 25° C. for 5 minutes, followed by a 15 minute wash at 50° C. with 2 times SSC containing 0.5% SDS; the final wash is with 1 times SSC containing 0.5% SDS at 50° C. for 15 minutes; filters are exposed to X-ray film (Kodak) overnight; of 106 clones screened, cDNA inserts are identified. The sequences of identified cDNAs may be determined, for example, by purifying plaques containing the cDNAs identified, and excising as phagemids according to the supplier's specifications, to generate insert-carrying Bluescript-SK variants of the phagemid vectors. Sequencing of the relevant clones across their entire sequence should reveal a putative ATG initiation codon together with an oligonucleotide of 5′ non-coding region and the coding region having a polyA splice site.

Once Sensin nucleic acids and/or polypeptides have been identified, mutations in these sequences can be readily identified by comparison to these “normal” sequences. DNA and polypeptide sequencing methods are well known to those of skill in the art. As noted above, a mutated Sensin polypeptide refers to a polypeptide that exhibits a deficiency in one or more characteristics displayed by “normal” Sensin proteins, such as an amino acid substitution, deletion, or insertion disrupting a zinc finger motif. Preferred mutated Sensin polypeptides exhibit a detectable phenotypic effect when expressed in cells or animals. Particularly preferred mutated Sensin polypeptides produce a recessive altered motor-related phenotype in an animal.

Mutations generated spontaneously may be identified by phenotypic effects that are known to relate to Sensin function (e.g., motor-related phenotypes seen in the Sensin mutation), and confirmed by direct sequencing of the putative mutant; by chromosome mapping; or by a combination of these methods. As an alternative to identifying spontaneous mutations in Sensin sequences, methods for introducing mutations into a known “normal” Sensin sequence can be performed. Such methods include site specific mutagenesis, in which a change in a nucleic acid sequence is introduced at a predetermined location in the sequence, and the nucleic acid is then introduced into a cell; gene transfer methods, in which a gene transfer vector is used to introduce a desired nucleic acid sequence into a cell genome; and “knockout” and “knockin” methods, in which a nucleic acid sequence is introduced into the genome of a cell at a specific location, often substituting an introduced gene for a genomic version (see, e.g., Kuhn, Science 269: 1427-9 (1995)).

In preferred embodiments, mutations may be induced by exposing a cell or whole animal to one or more mutagens, which randomly introduce mutations into the cell or animal genome. Suitable mutagens include, but are not limited to, radiation (gamma, beta, alpha, UV, etc.); base analogues such as bromouracil and aminopurine; chemicals such as nitrous acid, nitrosoguanidine, ethylnitrosourea (“ENU”), and ethylmethanesulfonate; intercalating agents such as acridine orange and ethidium bromide, to provide endogenous mutated Sensin nucleic acid sequences. ENU is preferred for use in whole animal, most preferably whole mouse, mutagenesis. See, e.g., Rinchik, Trends Genet. 7: 15-21 (1991). Protocols are available which provide a very efficient mutagenesis rate in mice. See, e.g., Favor, et al., Mut. Res. 231: 47-54 (1990).

The mutations induced in these manners may include deletions, substitutions, inversions, insertions, etc., however, the mutations recovered after ENU mutagenesis are mainly point mutations. Many of the mutants produced by ENU will therefore be hypomorphic (partial loss-of-function) mutations, although also gain-of-function as well as complete loss of function mutants can be expected. The frequency of mutant recovery from ENU methods is about 1/1000 for a specific locus that can be scored phenotypically, but strain, dosage and treatment regimen do influence the mutagenesis rate.

Methods for introducing or inducing mutations can be advantageously combined with nuclear transfer cloning methods to provide an animal comprising a mutated sequence of interest, e.g., by transferring the genetic material from a cell harboring the mutated sequence, preferably an embryonic stem cell in mice, into an enucleated oocyte; or with methods of blastocyst injection of genetically altered embryonic stem cells to provide germline chimeras; or with methods of pronuclear injection of fertilized mouse eggs with DNA constructs. See, e.g., Rideout et al., Cell 109: 17-27 (2002); Rideout et al., Nat. Genet. 24: 109-10 (2000); Nakao et al., Exp. Anim. 47:167-71 (1998). Alternatively, ENU can be used to mutagenize premeiotic spermatogonial stem cells in male animals, allowing the production of a large number of F1 founder animals from a single treated male.

Both dominant and recessive screens can be used to characterize mutations. Typically, male mice can be injected with ENU and then mated to females in order to produce G1 founders. These G1 mice can either be analyzed directly for dominant mutations or bred further to subsequently study recessive phenotypes. Very large numbers of mice can be analyzed in a dominant G1 screen. In this case, all G1 mice are screened for phenotypic abnormalities.

The screen for recessive mutations will involve at least two generations of breeding. From G1 founder males, G2 female offspring are raised, half of which are heterozygous for the newly induced mutations. Backcrossing G2 to the G1 founder male or intercrossing the G2 offspring is then carried out to provide homozygous G3 offspring. Recessive mutant phenotypes are then identified among the G3 offspring.

Manipulation of Novel Sensin Sequences

Techniques for the manipulation of nucleic acids, such as, e.g., subcloning, labeling probes (e.g., random-primer labeling using Klenow polymerase, nick translation, amplification), sequencing, hybridization and the like are well disclosed in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Nucleic acid sequences can be amplified as necessary for further use using amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1;29(11):E54-E54; Hafner et al., Biotechniques 2001 April;30(4):852-6, 858, 860 passim.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be performed by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The nucleic acids of the invention can also be provided in expression vectors and cloning vehicles, e.g., sequences encoding the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are disclosed, e.g., U.S. Pat. No. 5,426,039. To facilitate cloning of amplified sequences, restriction enzyme sites can be “built into” a PCR primer pair. Vectors may be introduced into a genome or into the cytoplasm or a nucleus of a cell and expressed by a variety of conventional techniques, well described in the scientific and patent literature. See, e.g., Roberts (1987) Nature 328:731; Schneider (1995) Protein Expr. Purif. 6435:10; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors may also be employed which exploit advantageous merits of each of the parent vector properties (See e.g., Feng (1997) Nature Biotechnology 15:866-870). Such viral genomes may be modified by recombinant DNA techniques to include the nucleic acids of the invention; and may be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof. See, e.g., U.S. Pat. Nos. 6,117,681; 6,107,478; 5,658,775; 5,449,614; Buchscher (1992) J. Virol. 66:2731-2739; Johann (1992) J. Virol. 66:1635-1640). Adeno-associated virus (MV)-based vectors can be used to transduce cells with target nucleic acids, e.g., in the in vitro production of nucleic acids and peptides, and in in vivo and ex vivo gene therapy procedures. See, e.g., U.S. Pat. Nos. 6,110,456; 5,474,935; Okada (1996) Gene Ther. 3:957-964.

The present invention also relates to fusion proteins, and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well disclosed in the scientific and patent literature, see e.g., Kroll (1993) DNA Cell. Biol., 12:441-53.

The nucleic acids and polypeptides of the invention can be bound to a solid support, e.g., for use in screening and diagnostic methods as described hereinafter. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of molecules to a solid support can be direct (i.e., the molecule contacts the solid support) or indirect (a “linker” is bound to the support and the molecule of interest binds to this linker). Molecules can be immobilized either covalently (e.g., utilizing single reactive thiol groups of cysteine residues (see, e.g., Colliuod (1993) Bioconjugate Chem. 4:528-536) or non-covalently but specifically (e.g., via immobilized antibodies (see, e.g., Schuhmann (1991) Adv. Mater. 3:388-391; Lu (1995) Anal. Chem. 67:83-87; the biotin/streptavidin system (see, e.g., Iwane (1997) Biophys. Biochem. Res. Comm. 230:76-80); metal chelating, e.g., Langmuir-Blodgett films (see, e.g., Ng (1995) Langmuir 11:4048-55); metal-chelating self-assembled monolayers (see, e.g., Sigal (1996) Anal. Chem. 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can also be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon (1989) Nature 377:525-531 (1989).

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Host Cells and Transformed Cells Comprising Novel Sensin Sequences

The invention also provides a transformed cell comprising a nucleic acid sequence of the invention, e.g., a sequence encoding a polypeptide of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

Vectors may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

For transient expression in mammalian cells, cDNA encoding a polypeptide of interest may be incorporated into a mammalian expression vector, e.g. pcDNA1, which is available commercially from Invitrogen Corporation (San Diego, Calif., U.S.A.; catalogue number V490-20). This is a multifunctional 4.2 kb plasmid vector designed for cDNA expression in eukaryotic systems, and cDNA analysis in prokaryotes; incorporated on the vector are the CMV promoter and enhancer, splice segment and polyadenylation signal, an SV40 and Polyoma virus origin of replication, and M13 origin to rescue single strand DNA for sequencing and mutagenesis, Sp6 and T7 RNA promoters for the production of sense and anti-sense RNA transcripts and a Col E1-like high copy plasmid origin. A polylinker is located appropriately downstream of the CMV promoter (and 3′ of the T7 promoter).

The cDNA insert may be first released from the above phagemid incorporated at appropriate restriction sites in the pcDNAI polylinker. Sequencing across the junctions may be performed to confirm proper insert orientation in pcDNAI. The resulting plasmid may then be introduced for transient expression into a selected mammalian cell host, for example, the monkey-derived, fibroblast like cells of the COS-1 lineage (available from the American Type Culture Collection, Rockville, Md. as ATCC CRL 1650).

For transient expression of the protein-encoding DNA, for example, COS-1 cells may be transfected with approximately 8 μg DNA per 106 COS cells, by DEAE-mediated DNA transfection and treated with chloroquine according to the procedures described by Sambrook et al, Molecular Cloning: A Laboratory Manual, 1989, Cold Spring Harbor Laboratory Press, Cold Spring Harbor N.Y., pp. 16.30-16.37. An exemplary method is as follows. Briefly, COS-1 cells are plated at a density of 5×106 cells/dish and then grown for 24 hours in FBS-supplemented DMEM/F12 medium. Medium is then removed and cells are washed in PBS and then in medium. A transfection solution containing DEAE dextran (0.4 mg/ml), 100 μM chloroquine, 10% NuSerum, DNA (0.4 mg/ml) in DMEM/F12 medium is then applied on the cells 10 ml volume. After incubation for 3 hours at 37° C., cells are washed in PBS and medium as just described and then shocked for 1 minute with 10% DMSO in DMEM/F12 medium. Cells are allowed to grow for 2-3 days in 10% FBS-supplemented medium, and at the end of incubation dishes are placed on ice, washed with ice cold PBS and then removed by scraping. Cells are then harvested by centrifugation at 1000 rpm for 10 minutes and the cellular pellet is frozen in liquid nitrogen, for subsequent use in protein expression. Northern blot analysis of a thawed aliquot of frozen cells may be used to confirm expression of receptor-encoding cDNA in cells under storage.

In a like manner, stably transfected cell lines can also prepared, for example, using two different cell types as host: CHO K1 and CHO Pro5. To construct these cell lines, cDNA coding for the relevant protein may be incorporated into the mammalian expression vector pRC/CMV (Invitrogen), which enables stable expression. Insertion at this site places the cDNA under the expression control of the cytomegalovirus promoter and upstream of the polyadenylation site and terminator of the bovine growth hormone gene, and into a vector background comprising the neomycin resistance gene (driven by the SV40 early promoter) as selectable marker.

An exemplary protocol to introduce plasmids constructed as described above is as follows. The host CHO cells are first seeded at a density of 5×10⁵ in 10% FBS-supplemented MEM medium. After growth for 24 hours, fresh medium is added to the plates and three hours later, the cells are transfected using the calcium phosphate-DNA co-precipitation procedure (Sambrook et al, supra). Briefly, 3 μg of DNA is mixed and incubated with buffered calcium solution for 10 minutes at room temperature. An equal volume of buffered phosphate solution is added and the suspension is incubated for 15 minutes at room temperature. Next, the incubated suspension is applied to the cells for 4 hours, removed and cells were shocked with medium containing 15% glycerol. Three minutes later, cells are washed with medium and incubated for 24 hours at normal growth conditions. Cells resistant to neomycin are selected in 10% FBS-supplemented alpha-MEM medium containing G418 (1 mg/ml). Individual colonies of G418-resistant cells are isolated about 2-3 weeks later, clonally selected and then propagated for assay purposes.

Sensin Binding Proteins and Substrates

The present invention provides methods for identifying polypeptides that bind to one or more Sensin polypeptides, typically through use of host cells expressing Sensin. These methods may include in vitro or in vivo characterization of proteins that by binding Sensin may modulate or be modulated by Sensin activity or expression levels. In particular, ubiquitination substrates of Sensin may be identified via these methods. The regulation of protein degradation of these substrates can be altered where there is an aberrant phenotype displayed by a cell, tissue, organ, or animal expressing Sensin polypeptides and/or mutated Sensin polypeptides.

Such identification methods include the yeast two-hybrid system in which Sensin full-length polypeptide or Sensin fragments including the C-terminal RING domain, N-terminal lysine-rich domain, or the sequence of SEQ. ID NO: 3 or 4 are expressed in yeast as “bait” fusion proteins in a screen against a cDNA library of “prey” fusion proteins. The fusion components of the screening system are typically the transactivation domain and DNA binding domain of a transcription factor such as yeast GAL4. When bait and prey bind each other, GAL4 transcriptional activation activity is reconstituted, upregulating transcription of a reporter gene construct. Such reporter constructs can be composed of GAL4 DNA binding sites upstream of a minimal promoter and marker gene such as lacZ, and library clones with increased reporter gene activity are identified by staining with βD-galactoside.

Binding proteins can also be identified by applying biochemical approaches to host cells expressing Sensin. In one embodiment, purified fusion protein consisting of Sensin polypeptides of the invention and a tag such as FLAG is incubated in vitro with lysate from a cell type or cell line of interest or candidate or test substrates under conditions that allow protein binding. Using antibodies against the FLAG tag, Sensin can then be pulled down together with any binding proteins and separated by protein gel electrophoresis or by other means such as mass spectrophotometry. Peptide microsequencing of the binding protein band can identify the binding protein by amino acid sequence.

In a preferred embodiment, an in vitro ubiquitination assay can be performed by including components of the ubiquitination pathway together with Sensin and substrate proteins under conditions that promote ubiquitination in vitro. Ubiquitinated proteins can then be isolated and identified by use of an anti-ubiquitin antibody and separation by electrophoresis or by other means. A host cell expressing Sensin proteins that is capable of mediating all or part of the ubiquitination pathway can be used to conduct similarly contemplated in vivo assays for ubiquitination.

Screening Methodologies

The present invention also provides methods for identifying compositions that affect one or more biochemical characteristics of Sensin polypeptides and/or mutated Sensin polypeptides, and/or that affect, and preferably ameliorate, an aberrant phenotype displayed by a cell, tissue, organ, or animal expressing Sensin polypeptides and/or mutated Sensin polypeptides.

These screening methods include target-based screening methods (i.e., screening of compositions for their activity at a protein or nucleic acid in vitro), cell-based screening methods, and whole animal-based screening methods. In practicing the methods of the invention, a variety of apparatus and methodologies can be used to in conjunction With the polypeptides and nucleic acids of the invention, e.g., to screen compositions that act as potential modulators of one or more of these characteristics.

In practicing the screening methods of the invention, a test compound can be contacted with a polypeptide of the invention in vitro or administered to a cell of the invention or an animal of the invention in vivo. Combinatorial chemical libraries are one means to assist in the generation of new chemical leads compounds.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250). Preparation and screening of combinatorial chemical libraries are well known to those of skill in the art, see, e.g., U.S. Pat. Nos. 6,004,617; 5,985,356. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), non-peptide peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem Tech, Louisville Ky., Symphonyl Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Suitable starting materials for preparing candidate test compounds can be derived from zinc finger-active compounds known in the art, such as those disclosed in U.S. Pat. Nos. 6,350,879; 6,242,478; and 5,929,114; Fujita et al., 3. Med. Chem. 19: 503-7 (1996); Rice et al., J. Med. Chem. 39: 3606-16 (1996); and Prasad et al., Bioorg. Med. Chem. 6: 1707-30 (1998). In particular, C-nitroso compounds and 2,2′-dithiobisbenzamides have been reported to act at certain zinc finger amino acids, and can release zinc from the fingers. Preferred starting materials for developing candidate compounds include: 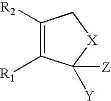
where X is O or S; Y is ═O; Z is an optionally present ═O; R₁ is C₁₋₆ alkyl, optionally substituted aryl (preferably optionally substituted phenyl), or cycloalkyl; R₂ is C₁₋₆ alkyl, optionally substituted aryl (preferably optionally substituted phenyl), cycloalkyl, or C(O)OR₃, where R₃ is C₁₋₆ alkyl or aryl. 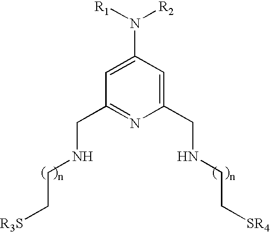
where R₁ and R₂ are independently H or C₁₋₆ alkyl; R₃ and R₄ are independently H, C₁₋₆ alkyl, or together form a disulfide bond; and n=1 or 2. 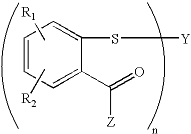
where R₁ and R₂ are independently hydrogen, halo, C₁₋₆ alkyl, cycloalkyl, hydroxy, nitro, cyano, heteroaryl, aryl, N(R₃)(R₄), C(O)OR₃, C(O)N(R₃)(R₄), SO₂R₃; R₃ and R₄ are independently hydrogen, NH₂, or C₁₋₆ alkyl; Z is halo, N(R₃)(R₄), heteroaryl, or aryl; and n=1 or 2, where when n=1, Y is H or a covalent bond to a second such structure.

Methods for identifying compounds active ubiquitin-mediated degradation pathways are well known in the art. See, e.g., U.S. Pat. Nos. 5,932,425; 6,060,262; and 6,368,809. Exemplary peptide-derived molecules are described in U.S. Pat. No. 5,932,425, column 8. Suitable starting materials for preparing candidate small molecule test compounds can also be derived from ubiquitin ligase-active compounds known in the art, such as those disclosed in Berleth, J. Biol. Chem. 267(23):16403-16411(1992); and Swinney, J. Biol. Chem. 277(26):23573-23581 (2002). In particular, arsenoxides have been reported to inhibit ubiquitin ligase activity. Preferred starting materials for developing candidate compounds include those cited within such references, and can include: 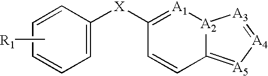
wherein R₁ is hydrogem, halo, C₁₋₆ alkyl, cycloalkyl, hydroxy, nitro, cyano, heteroaryl, N(R₃)(R₄), C(O)OR₃, C(O)N(R₃)(R₄), SO₂R₃, or a fused aryl or heteroaryl ring, where R₃ and R₄ are independently hydrogen, NH₂, or C₁₋₆ alkyl; 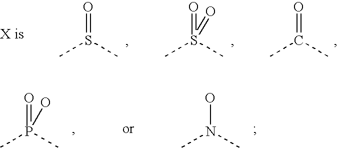
and A1-A5 are each independently N, C, or S.

Once suitable lead compounds have been identified, rational drug design methods can be used to optimize the utility of the compound as a biopharmaceutical agent. In these methods, 3-dimensional structure information obtained from x-ray crystallographic or NMR studies of a target polypeptide is used to specifically produce or modify a therapeutic agent to interact more specifically and/or effectively with the wild-type protein target, thus increasing the therapeutic efficacy of the parental drug and/or decreasing non-specific, potentially deleterious interactions. See, e.g., Hicks, Curr. Med. Chem. 8: 627-50 (2001); Gane and Dean, Curr. Opin. Struct. Biol. 10: 401-4 (2000).

The cell- and animal-based screening methods of the present invention can also be used to assess compositions for their ability to inhibit expression or function of a target polypeptide, thereby affecting, and preferably ameliorating, an aberrant phenotype displayed by an animal expressing mutated Sensin polypeptides. These compositions can include antisense oligonucleotides, RNAi, ribozymes, decoy oligonucleotides, etc.

Antisense oligonucleotides capable of binding polypeptide message can inhibit polypeptide activity by targeting mRNA. Strategies for designing antisense oligonucleotides are well disclosed in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198. Antisense oligonucleotides have been applied to inhibiting the expression of zinc finger proteins. See, e.g., Bavisotto et al., J. Exp. Med. 174: 1097-1101 (1991).

Naturally occurring nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as disclosed in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold (1995) J. of Biol. Chem. 270:13581-13584).

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are disclosed by Rossi (1992) Aids Research and Human Retroviruses 8:183; hairpin motifs by Hampel (1989) Biochemistry 28:4929, and Hampel (1990) Nucl. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNAi, or “posttranslational gene silencing” refers to methods by which double-stranded RNA molecules trigger a gene silencing response in various cells. In these methods, soluble-stranded RNA molecules are reduced to small interfering RNAs (“siRNAs”), preferably about 21-23 nucleotides in length, by endogenous nucleases. Methods have been disclosed for the design of RNAi oligonucleotides to provide sequence-specific gene silencing. See, e.g., Elbashir et al., Nature 411: 494-8 (2001). The RNAi phenomenon differs from antisense methods, in that it is mediated by double-stranded RNA rather than by single-stranded antisense RNA. Its use has been demonstrated in cells as diverse as those from the nematode C. elegans to numerous mammalian cell types.

RNAi oligonucleotides may be provided to cells either as presynthesized (by either in vitro or in vivo methods) double-stranded RNA molecules, and/or by expressing the RNAi oligonucleotide directly in target cells. For expression of siRNAs within cells, some researchers engineered plasmid vectors that contained either the polymerase III H1-RNA, or U6 promoter, a cloning site for the stem-looped RNA insert, and a 4-5-thymidine transcription termination signal. The inserts were ˜50 nt, with ˜20 nt inverted repeats (coding for the dsRNA stem complementary to a target gene) and ˜10 nt spacers (coding for the loop). Polymerase III promoters were chosen because these promoters generally have well-defined initiation and stop sites and their transcripts lack poly(A) tails. The termination signal for these promoters is defined by 5 thymidines, and the transcript is typically cleaved after the second uridine. Cleavage at this position generates a 3′ UU overhang in the expressed siRNA, which is similar to the 3′ overhangs of synthetic siRNAs. In another approach, U6 promoter-driven expression vectors were made that expressed the sense and antisense strands of siRNAs. Upon expression, these strands presumably anneal in vivo to produce the functional siRNAs. See, e.g., Brummelkamp, et al., Science 296: 550-3 (2002); Paddison et al., Genes and Dev. 16: 948-58 (2002); Paul, et al., Nature Biotechnol. 20: 505-8 (2002); Sui, et al., Proc. Natl. Acad. Sci. USA 99: 5515-20 (2002). Yu, et al., Proc. Natl. Acad. Sci. USA 99: 6047-52 (2002); Miyagishi and Taira, Nature Biotechnol. 20: 497-500 (2002); and Lee, et al., Nature Biotechnol. 20: 500-5 (2002).

“Decoy oligonucleotides” refer to double stranded nucleic acids that bind to a DNA binding protein, thereby preventing binding of the DNA binding protein to its natural target in the cell. Transfection of cis-element double stranded (ds) decoy oligonucleotides has been reported as a powerful tool for gene therapy. See, e.g., Tomita, et al., Exp. Nephrol., 5429-434 (1997). The decoy approach may also enable us to treat diseases by modulation of endogenous transcriptional regulation as a “loss of function” approach at the pre-transcriptional and transcriptional levels in a similar fashion to employing antisense technology as a “loss of function” approach at the transcriptional and translational levels.

Antibodies

The present invention also provides isolated or recombinant antibodies that specifically bind to a polypeptide of the invention. Such antibodies can be used to isolate, identify or quantify a polypeptide of the invention or related polypeptides; and/or as all or part of a therapeutic compositions.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and disclosed in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies which bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques disclosed for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention may be used in screening for similar polypeptides from other organisms and samples. In such techniques, polypeptides from the organism are contacted with the antibody and those polypeptides which specifically bind the antibody are detected. Any of the procedures described above may be used to detect antibody binding.

Formulation and Administration of Pharmaceutical Compositions

The various compositions identified or provided according to the foregoing methods may be formulated as pharmaceutical compositions comprising small molecules, nucleic acids, vectors, antibodies and/or polypeptides of the invention. These may preferably be provided to a subject in need thereof, e.g., for treatment or prophylaxis of a disease caused by or related to mutated Sensin polypeptides. As noted herein, animals comprising the mutated nucleic acids and polypeptides of the present invention exhibit altered motor-related phenotypes. Subjects in need of the pharmaceutical compositions of the present invention include those exhibiting altered levels (e.g., decreases or increases in proliferation) or characteristics (e.g., losses in morphology, function or normal cellular content) in neurons or glial cells as compared to normal “control” animals. Diseases related to such cells include neurodegenerative disorders such as Parkinson disease, Alzheimer disease, Multiple Sclerosis, Amyotrophic Lateral Sclerosis (ALS), and various sensory neuropathies, etc.

The function of Sensin polypeptides in a subject may be subject to manipulation in a variety of ways to produce a therapeutic effect. For example, people exhibiting impaired motor function may be appropriate candidates for therapies that target an increase in the amount or function of Sensin polypeptides, thereby altering the phenotype of the subject.

Molecules for therapeutic and/or prophylactic uses can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are disclosed in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions of the invention, when administered orally, can be protected from digestion. This can be accomplished either by complexing, e.g., a nucleic acid, peptide or polypeptide with additional components in a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix (1996) Pharm Res. 13:1760-1764; Samanen (1996) J. Pharm. Pharmacol. 48:119-135; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani (1996) “Systemic delivery of peptides and proteins across absorptive mucosae” Crit. Rev. Ther. Drug Carrier Syst. 13:85-184. For topical, transdermal administration, the agents are formulated into ointments, creams, salves, powders and gels. Transdermal delivery systems can also include, e.g., patches.

Compositions of the invention can also be administered in sustained delivery or sustained release mechanisms, which can deliver the formulation internally. For example, biodegradable microspheres or capsules or other biodegradable polymer configurations capable of sustained delivery of a peptide can be included in the formulations of the invention (see, e.g., Putney (1998) Nat. Biotechnol. 16:153-157).

For inhalation, the nucleic acids, peptides or polypeptides of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Compositions of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intratracheal (e.g., by aerosol) or transmucosal (e.g., buccal, bladder, vaginal, uterine, rectal, nasal mucosa). Actual methods for preparing administrable compositions will be known or apparent to those skilled in the art and are disclosed in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun (1997) Anesth Analg. 85:317-323). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are disclosed in detail, in e.g., Remington's, See also, Bai (1997) J. Neuroimmunol. 80:65-75; Warren (1997) J. Neurol. Sci. 152:31-38; Tonegawa (1997) J. Exp. Med. 186:507-515.

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky (1995) Bioconjug. Chem. 6:705-708). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla (1996) J. Pharm. Sci. 85:5-8), transmucosally, or orally. The invention also provides pharmaceutical preparations in which the nucleic acid, peptides and/or polypeptides of the invention are incorporated within micelles and/or liposomes (see, e.g., Suntres (1994) J. Pharm. Pharmacol. 46:23-28; Woodle (1992) Pharm. Res. 9:260-265). Liposomes and liposomal formulations can be prepared according to standard methods and are also well known in the art, see, e.g., Remington's; Akimaru (1995) Cytokines Mol. Ther. 1:197-210; Alving (1995) Immunol. Rev. 145:5-31; Szoka (1980) Ann. Rev. Biophys. Bioeng. 9:467, U.S. Pat. Nos. 4,235,871, 4,501,728 and 4,837,028.

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton (1997) “Bioavailability and transport of peptides and peptide drugs into the brain” Peptides 18:1431-1439; Langer (1990) Science 249:1527-1533.

Methods for Detecting Novel Sensin Sequences in Samples from Subjects

Subjects, e.g., humans or animals considered at risk for the presence of a mutated Sensin sequence, can be screened for the occurrence of such mutations using many of the compositions described herein. The presence of mutated Sensin nucleic acid and/or protein sequences may be identified by numerous methods known to those of skill in the art, such as NMR, spectrophotometry, radiography, electrophoresis, capillary electrophoresis, high performance liquid chromatography (HPLC), thin layer chromatography (TLC), various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Examples of suitable samples include those obtained from cells, a biological fluid (such as blood, plasma, serum, urine, bile, saliva, tears, cerebrospinal fluid, aqueous or vitreous humor, or any bodily secretion), a transudate or exudate (e.g., fluid obtained from an abscess or other site of infection or inflammation), a fluid obtained from a joint (e.g., a normal joint or a joint affected by disease such as rheumatoid arthritis, osteoarthritis, gout or septic arthritis), or the like. Preferred biological samples of the present invention are blood, plasma, or serum.

Samples may be obtained from any organ or tissue (including a biopsy or autopsy specimen) or may comprise cells (including primary cells, passaged or cultured primary cells, cell lines, cells conditioned by a specific medium) or medium conditioned by cells. In preferred embodiments, a biological sample is free of intact cells. If desired, the biological sample may be subjected to prior processing, such as lysis, extraction, subcellular fractionation, and the like. See, Deutscher (ed.), Meth. Enzymol. 182:147-238 (1990).

Such methods can be used not only for diagnosis and for prognosis of existing disease, but may also be used to predict the likelihood of the future occurrence of disease, for selecting subjects for inclusion in a clinical trial, or for assessing the effectiveness of a particular treatment regimen.

Animals Comprising Novel Sensin Sequences

The present invention also relates to animals, one or more cells of which comprise a Sensin nucleic acid not ordinarily present in an animal of that species. Such animals might comprise, for example, a Sensin nucleic acid, or a mutated form thereof, obtained from one species and introduced into an animal of a different species, such as a nucleic acid encoding a human Sensin protein inserted into a mouse or vice versa. Alternatively, a nucleic acid encoding a human Sensin protein, or a mutated form thereof, may be inserted into a human, to alter the gene dosage of that particular protein.

In another alternative, endogenous Sensin nucleic acid sequences in an animal may be mutated, e.g., using mutagens such as: radiation (gamma, beta, alpha, UV, etc.); base analogues such as bromouracil and aminopurine; chemicals such as nitrous acid, nitrosoguanidine, ethylnitrosourea, and ethylmethanesulfonate; intercalating agents such as acridine orange and ethidium bromide, as described above to provide animals, one or more cells of which comprise mutated Sensin nucleic acid sequences.

Preferred animal species include primate, caprine, bovine, ovine, porcine, and murine species. Most preferred are mice, due to their small size and (relatively) short gestational period and lifespan, which provide the ability to perform multiple rounds of breeding, while maintaining a relatively small animal facility.

The sequences used to provide transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,258,998; 6,211,428; 6,187,992; 6,156,952; 6,118,044; 6,111,166; 6,107,543; 6,107,541; 6,011,197; 5,959,171; 5,945,577; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., U.S. Pat. No. 6,395,958, and Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, discloses making and using transgenic non-human mammals which express in their brains a nucleic acid construct comprising a DNA sequence. Rideout et al., Cell 109: 17-27 (2002); and Rideout et al., Nat. Genet. 24: 109-10 (2000) disclose nuclear transfer cloning methods may be used to provide animals comprising a mutated sequence of interest, e.g., by transferring the genetic material from a cell harboring the mutated sequence, preferably an embryonic stem cell in mice, into an enucleated oocyte. See, e.g., U.S. Pat. No. 5,387,742, discloses injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, discloses making and using a transgenic mouse whose genome comprises a disruption of the gene encoding amyloid precursor protein (APP).

The animals of the present invention may be used for a variety of purposes, including the production of proteins encoded by expression constructs as described herein, screening methods to identify modulators of Sensin polypeptides, testing the effects of such modulators on altered motor-related phenotypes, identifying genetic modulators of altered motor-related phenotypes, etc.

In certain embodiments, such animals are used in sensitized genetic screens. Sensitized screens allow the identification of additional sequences in an animal that relate in some fashion to a gene of interest. In particular, genes and their expressed products that ameliorate or worsen one or more phenotypic characteristics of the mutated Sensin sequences of the present invention can be identified. Such methods can comprise screening for additional mutations on a sensitized genetic background, where the sensitized background exhibits an altered motor-related phenotype. Genes that are related to such phenotypes may be readily identified using such screens.

The methods described herein for generating and identifying novel Sensin sequences, such as ENU mutagenesis methods, knockout generation, knockin generation, etc., can be applied to such sensitized animals to identify mutations upstream from a mutated Sensin polypeptide in a pathway, downstream in a pathway, or additional mutations in Sensin proteins, that affect a phenotype of interest. As discussed above, both dominant and recessive breeding screens can be used to characterize and identify such mutations.

### EXAMPLES

### Example 1

### ENU Mutagenesis

Male B6 were mutatagenized with 3 doses of 85 mg ENU (Ethyl nitroso urea)/kg body weight by intra-peritoneal injection. After regaining fertility (approximately 3 months) the mutagenized mice (termed G0) were bred with wild type B6 female mice to produce G1 founder male offspring. The G1 males were bred with wildtype B6 female mice, the offspring referred to as G2 mice. G2 female mice were bred with the G1 male parent to produce approximately 20 G3 offspring. The G3 offspring were phenotyped for outlier mutants. A mutant pedigree was identified when one or more of the G3 offspring exhibit a phenotype not seen in wild-type mice, where a pedigree is a series of G3 mice derived from the same G1 male parent. On identification of a mutant pedigree, the mutation were maintained by breeding to B6 wild type mice, and were mapped by outcrossing to another mouse strain, where the offspring from this cross are intercrossed and the offspring are phenotyped and genotyped.

### Example 2

### Phenotypic Screening of Mutant Mice

In a library bred from ENU-treated C57BL/63 (B6) mice, a third generation male offspring developed tetanic paralysis at 4 months of age. A mutant breeding line, Senseless, had been established by backcrossing this founder to normal B6 mice. Adult-onset paralysis was inherited as a single recessive Mendelian trait.

### Example 3

### Chromosomal Mapping of Mutations

To map the chromosomal location of the Senseless mutation, B6 Senseless animals were backcrossed to NOD.H2k congenic mice. Single nucleotide polymorphism typing of tail DNA from N2 animals that developed paralysis (n=30) revealed linkage to distal chromosome 16. The recombinational breakpoints in these animals was further delimited using markers between D16Mit25 and D16Mit86, positioning the Senseless mutation within a seven megabase (Mb) region between D16Mit70 and D16Mit86. Novel polymorphic markers were identified to further narrow the region containing the Senseless mutation to a 3.6 Mb region that was predicted to contain exons from 39 genes. While a large complex of 27 keratin- and keratin associated protein-related genes was not considered likely to be associated with the Senseless phenotype, there were at least five genes with evidence for expression in the nervous system, making them candidates for the senseless gene.

### Example 4

### Identification of Senseless Mutation

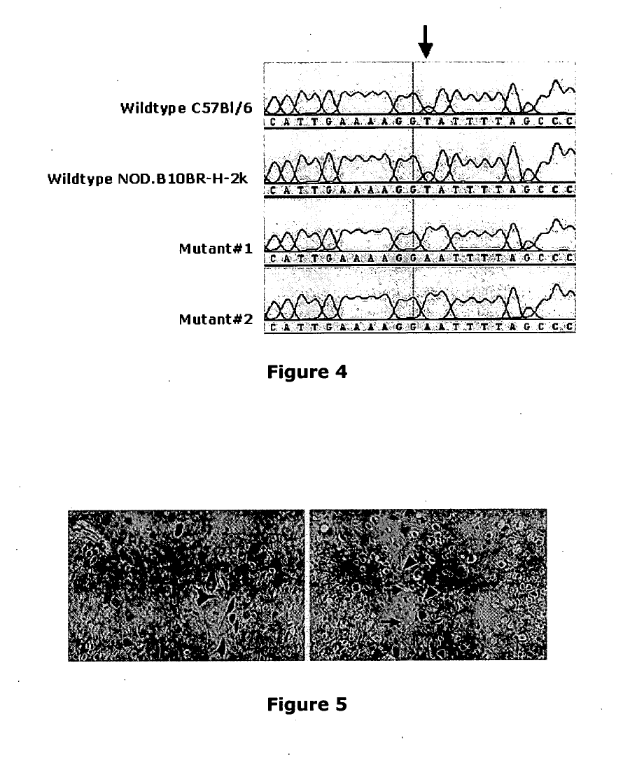

We designed primers to PCR amplify and sequence genomic DNA covering splice sites and exons of ten non-keratin related genes. A single base change was identified in the sequence immediately following exon 11 of the Sensin gene, where the nucleotide ‘T’ had been mutated to ‘A’ (FIG. 4).

### Example 5

### Histological Characterization of Mutations

Histology examination of samples from Senseless mice indicates signs of gliosis in the neuropil of the spinal cord as well as excessive vacuoles, both symptoms with similarity to histopathology of neurodegenerative disease, particularly ALS which affects spinal cord motor neurons (FIG. 5).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

While the invention has been described in detail with reference to certain preferred embodiments thereof, it will be understood that modifications and variations are within the spirit and scope of that which is described and claimed.

1. A non-human mammalian animal comprising a non-naturally occurring mutation in a sensin gene.

2. A non-human mammalian animal according to claim 1, wherein the resulting mutated sensin gene causes a deficit in a motor-related phenotype in said mammalian animal.

3. A non-human mammalian animal according to claim 1, wherein said mutated sensin gene is expressed as a cDNA having substantial sequence homology with SEQ ID NO: 1.

4. A non-human mammalian animal according to claim 1, wherein said mutated sensin gene is expressed as a cDNA having substantial sequence homology with SEQ ID NO: 2.

5. A non-human mammalian animal according to claim 1, wherein said mutated sensin gene comprises a mutation in a splice site.

6. A non-human mammalian animal according to claim 5, wherein said mutation in a splice site is a single nucleotide change in a splice donor site.

7. A non-human mammalian animal according to claim 6, wherein said splice donor site is GA.

8. A non-human mammalian animal according to claim 6, wherein said mutation in a splice site results in deletion of an exon from a cDNA expressed from said gene.

9. A non-human mammalian animal according to claim 8, wherein said cDNA is expressed as a protein comprising a deletion of the sequence KEDLKWSSLLQVIE (SEQ ID NO: 3) or KVDLKWNSLLKIIE (SEQ ID NO: 4).

10. A non-human mammalian animal according to claim 1, wherein said one or more mammals are mice expressing a cDNA encoding the protein of SEQ ID NO: 5.

11. A non-human mammalian animal according to claim 1, wherein said mammalian animal is a mouse expressing a cDNA encoding the protein of SEQ ID NO: 6.

12. A non-human mammalian animal according to claim 1, wherein said mouse is homozygous for said non-naturally occurring mutation in a sensin gene.

13. A colony comprising a plurality of non-human mammalian animals comprising an identical non-naturally occurring mutation in a sensin gene.

14. A method for screening for modulators of motor activity in a mammal, comprising:
contacting one or more mammals with one or more test compounds, and
determining whether said test compound(s) alter(s) a sensin-mediated motor-related phenotype, thereby identifying said modulator(s).

15. A method according to claim 14, wherein said one or more mammals comprise a mutation in a sensin gene, wherein the resulting mutated sensin gene causes a deficit in a motor-related phenotype in said one or more mammals.

16. A method according to claim 14, wherein said one or more test compounds are nucleic acids.

17. A method according to claim 16, wherein said nucleic acids are antisense molecules.

18. A method according to claim 16, wherein said nucleic acids are RNAi molecules.

19. A method according to claim 15, wherein said mutated sensin gene is expressed as a cDNA having substantial sequence homology with SEQ ID NO: 1.

20. A method according to claim 15, wherein said mutated sensin gene is expressed as a cDNA having substantial sequence homology with SEQ ID NO: 2.

21. A method according to claim 15, wherein said mutated sensin gene comprises a mutation in a splice site.

22. A method according to claim 21, wherein said mutation in a splice site is a single nucleotide change in a splice donor site.

23. A method according to claim 22, wherein said splice donor site is GA.

24. A method according to claim 22, wherein said mutation in a splice site results in deletion of an exon from a cDNA expressed from said gene.

25. A method according to claim 24, wherein said cDNA is expressed as a protein comprising a deletion of the sequence KEDLKWSSLLQVIE (SEQ ID NO: 3) or KVDLKWNSLLKIIE (SEQ ID NO: 4).

26. A method according to claim 15, wherein said one or more mammals are mice expressing a cDNA encoding the protein of SEQ ID NO: 5.

27. A method according to claim 15, wherein said one or more mammals are mice expressing a cDNA encoding the protein of SEQ ID NO: 6.

28. A method for screening for modulators of motor activity in a mammal, comprising:
contacting one or more mammals with one or more test compounds, and
determining whether said test compound(s) alter(s) expression of a mutated sensin gene, or alter(s) expression of a wild type sensin gene, thereby identifying said modulator(s).

29. A method according to claim 28, wherein said one or more test compounds are nucleic acids.

30. A method according to claim 29, wherein said nucleic acids are antisense molecules.

31. A method according to claim 29, wherein said nucleic acids are RNAi molecules.

32. A method according to claim 28, wherein said mutated sensin gene is expressed as a cDNA having substantial sequence homology with SEQ ID NO: 1.

33. A method according to claim 28, wherein said mutated sensin gene is expressed as a cDNA having substantial sequence homology with SEQ ID NO: 2.

34. A method according to claim 28, wherein said mutated sensin gene comprises a mutation in a splice site.

35. A method according to claim 34, wherein said mutation in a splice site is a single nucleotide change in a splice donor site.

36. A method according to claim 35, wherein said splice donor site is GA.

37. A method according to claim 35, wherein said mutation in a splice site results in deletion of an exon from a cDNA expressed from said gene.

38. A method according to claim 37, wherein said cDNA is expressed as a protein comprising a deletion of the sequence KEDLKWSSLLQVIE (SEQ ID NO: 3) or KVDLKWNSLLKIIE (SEQ ID NO: 4).

39. A method according to claim 28, wherein said one or more mammals are mice expressing a cDNA encoding the protein of SEQ ID NO: 5.

40. A method according to claim 28, wherein said one or more mammals are mice expressing a cDNA encoding the protein of SEQ ID NO: 6.

41. A method comprising:
contacting a subject in need thereof with one or more compounds in an amount sufficient to alter a motor activity in said subject, wherein said modulators of motor activity alter a sensin-mediated motor-related phenotype in a mammalian animal.

42. A method according to claim 41, wherein said subject comprises a mutation in a sensin gene.

43. A method according to claim 41, wherein said mammalian animal is a mouse expressing the protein of SEQ ID NO: 5.

44. A method according to claim 41, wherein said non-human mammalian animal is a mouse expressing the protein of SEQ ID NO: 6.

45. A method for determining the level of a sensin polypeptide in a sample, comprising:
assaying for said sensin polypeptide with an antibody, or fragment thereof, that specifically binds to said sensin polypeptide.

46. A method according to claim 45, wherein said antibody binds to the protein of SEQ ID NO: 5.

47. A method according to claim 45, wherein said antibody binds to the protein of SEQ ID NO: 6.

48. A method according to claim 45, wherein said antibody comprises an affinity for a mutated sensin polypeptide greater than an affinity for a wild type sensin polypeptide.

49. A method for diagnosing a motor deficit in a subject, comprising:
determining whether said subject comprises a mutation in a sensin nucleic acid sequence.

50. A method according to claim 49, wherein said nucleic acid sequence is selected from the group consisting of a genomic DNA sequence and an expressed RNA sequence.

51. A method for identifying a subject suitable for treatment with an one or more compounds having an effect on a motor deficit, comprising:
determining whether said subject comprises a mutation in a sensin nucleic acid sequence, wherein the presence of said mutation identifies said subject for inclusion or exclusion from said treatment.

52. A kit for determining whether a sample comprises a mutation in a sensin nucleic acid sequence, comprising:
an assay component that detects a mutation in a sensin nucleic acid sequence; and
instructions for performing said determination.

53. An isolated sensin polypeptide.

54. An isolated polypeptide comprising at least 15 consecutive amino acid residues of:
SEQ ID NO: 5;
SEQ ID NO: 6;
SEQ ID NO: 7;
SEQ ID NO: 8;
SEQ ID NO: 9;
SEQ ID NO: 10; or
SEQ ID NO: 11.

55. An isolated polypeptide comprising the sequence of SEQ ID NO: 3 or SEQ ID NO: 4.

56. An isolated sensin nucleic acid.

57. An isolated nucleic acid molecule encoding a polypeptide comprising at least 15 consecutive amino acid residues of SEQ ID NO: 5;
SEQ ID NO: 6;
SEQ ID NO: 7;
SEQ ID NO: 8;
SEQ ID NO: 9;
SEQ ID NO: 10; or
SEQ ID NO: 11.

58. An isolated nucleic acid molecule encoding the sequence of SEQ ID NO: 3 or SEQ ID NO: 4.

59. A recombinant vector comprising a nucleic acid sequence encoding a polypeptide comprising at least 15 consecutive amino acid residues of SEQ ID NO: 5, SEQ ID NO: 6, SEQ ID NO: 7, SEQ ID NO: 8, SEQ ID NO: 9, SEQ ID NO: 10, or SEQ ID NO: 11; or a nucleic acid sequence encoding a polypeptide having the sequence of SEQ ID NO: 3 or SEQ ID NO: 4.

60. A recombinant vector according to claim 59, wherein said vector is an expression vector adapted for expression of a eukaryotic coding sequence.

61. A recombinant cell comprising the recombinant vector of claim 59.

62. An antibody that specifically binds to a sensin polypeptide.

63. A method of identifying one or more genes related to mammalian motor function, comprising:
introducing one or more mutations into the genome of a mouse comprising a nucleic acid encoding a mutated sensin gene, wherein the mutated sensin gene causes a deficit in a motor-related phenotype in said mouse; and
identifying mutations that affect said sensin-mediated motor-related phenotype of said mouse.

64. A method comprising:
expressing in one or more cells of a subject in need thereof an sensin polypeptide encoded by an expression construct in which an sensin nucleic acid is operably inserted downstream in the direction of transcription of a transcriptional regulatory region functional in said one or more cells.

65. A method according to claim 64, wherein a deficit in a motor-related phenotype is altered in said subject following said expressing step.

66. A method according to claim 64, wherein said expression construct is introduced into one or more cells ex vivo, and said cells are subsequently administered to said subject.

67. A method of making a non-human mammalian animal, comprising:
introducing one or more mutations into a sensin gene of said non-human mammalian animal.

68. A method of identifying one or more genes related to motor function, comprising:
determining whether said genes encode polypeptides that bind to an isolated sensin polypeptide.


SEQUENCE LISTING (84 sequences)

SEQ ID NO: 1 - DNA, 5454 bp, Mus musculus
cttcggctctgcagactgagagccgggctgccaccgccaccatgggcgggaagaacaagcagcggactaaggggaacctcaggccttcaaacagtggccgagctgcagaactcctcgccaaagagcagggaacagtgcctgggttcattggttttggcacatctcacagtgacctgggctatgttccggctgttcaaggcgcggaagacatagacagtcttgtagattccgatttccgaatggtgctgcggaaactttccaaaaaagatgttacaacaaagctaaaggcaatgcaagaatttggaattatgtgcacagagagagacacagaagccgtcaaaggggttcttccatactggccaagaatcttctgcaaaatctcccttgatcatgatcgccgtgttcgagaggcgacgcagcaagcttttgaaaaacttatccttaaagtaaagaagcacttagctccctatttaaaaagcgtgatgggctattggttgatggctcagtgtgacacatatccaccagctgcactggcagcaaaagatgcattcgaagccgctttccctccaagcaagcaacctgaagccatagcgttttgcaaggaagaaattacaactgtgttacaggaccatcttctgaaggagactcctgacacactcagtgaccctcaaactgtgccagaggaagagagagaggctaagttccaccgagtcgtgacgtgctctttattggcactgaagagattgctttgtttcttacctaacaatgagcttgattctctggaggagaaatttaaatcacttttatcacagaataaattttggaagtatggaaaacacagtgtacctcaggtccgctcagcatattttgagttagtttctgctttgtgccagcacgttccccaggtgatgaaagaggaagctgccaaagtgagtccatctgtcctgctcagcattgatgacagtgaccctgtggtctgcccagctctctgggaggctgtgctctacacgctgacaactattgaggactgttggtttcatgtaaatgccaagaaaagtgtgtttccgaagctgatggccatgatccgagagggtggccggggcctagctgctgttatgtatccttacctcttaccgttcatcagcaaactccctcagtccatcacagagccaaagctggacttcttcaaaaacttcctcacctctctagttaccgggctgtcaactgagaggaccaaatcgagctcttctgagtgctcagctgtcatcccggcattttttgaatgtttgcggtttataatgcagcagaacttaggcgaggaggagatggtgcagatgcttatcaatgagcagttgatcccatttattgatacagtcctcaaagactcaggactgcaccacggaccgatgtttgaccatttagcagacacattgagctcttgggaagccaaagcagatgcagagcgagaccctggagctgtttacaacttggagaacgttctgctaagcttctggggaagactgtcagagatctgcaccgagaaaatccgccagccagaagcagatgtgaagtccgttctgtgtgtgtctagcctagtaggggtccttcagaggccaaggagctcactggagttacacagaaagaaaactgctcaggtcaggtttgccatcaatatacctgaagctcataaaggggatgaaaagtccatgtcttcagaaggagagaacagcgagggctctgacggaggggctcagtctcctctcagtaatacatcttcagacttagtatctcctttaaggaaaaaacctctggaagacttagtctgtaagctggcagaggtgagcattagctttgtcaacgagcggaagtcagagcagcacctgcagtttctctccacgctgctcgactccttctcctccgtccaagtatttaacattctcctcagtgacaaacagaagaatgtcgtcaaagccaaacctctggaaataaccaaacttgcagaaaaaaatcctgcagttaagttcttatatcataaattgataggctggctaaatgacagccagaaagaggacgggggcttcctggtggacatcttgtatagtgccctccgatgctgtgacagtggtgtggaaaggaaagaagtcttggatgatctaaccaaggaggacctgaagtggagttctcttcttcaggtcattgaaaaggcatgttctagctcagataaacatgctttagtaactccttggctaaaaggcagtattcttggagagaaattggttgccttggcagactgtctttgtgataaggacttggaagccacaacatctgaatcccactcatcagaacagtggagtctgctaagactggcgttatcccaacatgtcaaaaacgattacttgattggagaagtatacgttggaagaattattgttaaacttcatgaaactttatctaaaacaaaggatttatcagaagcggcaaacagtgactcttcagtgtcttttgtctgtgatgtggtccatagcttcttcagttcagcaggaggaggcttgctaatgccaccatctgaagacttgttattaactctctttcagttatgcgctcagagcaaagaacggacacacttgccagattttctgatctgtaaactgaagaatactttgctttctggtgtaaatttattggtccatcaaactgcaagtacatatgagcagagtaccttcctacgtttgtctgttctgtggctgaaggaccaggttcagtcttcggctttggataacacaagtcttcaggtcctcttgtctgctgctggcgacttgctgggtactcttgtagagagtgaggacacgtctcttcttggagtttatatcggaagtgtaatgcccagtgacagcgagtgggaaaagatgaggcaggctcttcctgttcagtggttacacagacctcttttagagggaaggctgagtttaaattatgaatgcttcaagacagattttaaggaacaagacacaaagactcttcccaaccatctgtgtacttcatcgttattgagcaaaatgatcttagttgcccaaaaaaagaaattagtcctagaagacaacgtccttgaaaaaataattgcagagctgctgtattcactgcagtggtgtgaagaactcgacaatgcgccatctttcctaagtggattttgtggaatccttcaaaaaatgaatataacttacagcaacttatctgtacttagtgagacttctagcctcttgcagctgttatttgacagatctagaaaaaatggcacactttggtctcttattattgctaagttgatcctttcccgaagtatttcatctgatgaagtgaaaccatactataaaagaaaagaaagtttcttccctctaacagaaggtagtttgcataccattcaaagtctctgtccattcttgtctaaagaagagaagaaggaatttagtgctcacagtatacccgcttttttgggttggactaaagaagacctttgcagtattaatggagcctttggacatcttgccattttcaattcttgtctgcaaaccagaagtatagacgacaaacagctattgcatggcatattaaaaattataacaagctggaggaaacagcatgaagatatttttctttttagctgtaatctgtcggaagcgagtccagaggtccttggtttaaacatagagatcatgcggttcctttccttgtttctcaagcactgcgcgtacccgctcccgctggcagacagtgaatgggacttcatcatgtgctccatgctggcttggttggagacaaccagtgagaaccaggctctgtactctgttccacttgtgcagctgtttgcctgtgtcagctttgatctggcctgtgatctctgtgccttttttgactcaataactccagatattgttgacaatcttcctgtaaatctcatcagtgagtggaaagagtttttttctaaaggcatccacagtttgctattacctcttttggtaaatgctatcggagaaaacaaagacctatctgaaacgtcctttcagaacgcaatgctgaaacccatgtgtgaaacactaacatacatctccaaggaccagctactgagccacaagctccctgcgagattggttgccagccagaaaacaaacttgccagagcacctccagactctgctgaacactttgaccccactgcttctcttcagagccagacctgtgcaaattgctgcttatcatatgctgtgcaaactgatgcctgaattgccacagcatgatcaggacaatctgaggtcgtatggagatgaagaggaagaaccagccttgtcgccgccggccgcgctgatgtctctcctcagctctcaggaggagctgctggagaatgtcctgggctgtgtccctgtgggccagatcgtgaccgttaagccactgagcgaggacttctgctatgtcctgggatacctcctcacttggaagttaatactgactttcttcaaagctgcatcgtctcagcttcgtgctctgtattcaatgtaccttcggaaaacaaagagtctgaataaattactctatcatctcttcagacttatgccagaaaaccctacgtacggagagacagctattgaggtatcaagtaaagaccccaagaccttcttcaccgaggaggttcagctgagtattagagaaacagcaactcttccgtatcatatcccacacctggcgtgctcggtctatcacatgactttaaaagacttgcctgccatggttaggctatggtggaatagcagtgagaagcgtgtcttcaatattgtagatagatttacaagcaagtatgtcagcaatgttctttcttttcaagaaatatcttctgtacaaacaagtacacagctattcaatggcatgacggttaaggcgcgagctactactcgagaagtgatggctacgtacaccatcgaagacatagtcattgaactcataatacagttgccttccaattacccactgggctcaataacagtggaaagtgggaagaggatcggggtggctgtgcagcagtggcgaaactggatgctgcagctgagcacgtacctcactcaccagaacggaagtatcatggaaggcttagcattatggaagaataatgtagacaaacggtttgaaggtgttgaagattgtatgatctgcttctcagttattcatggtttcaactattctcttcccaaaaaagcctgtagaacatgcaagaaaaagtttcactcagcttgcctgtacaaatggtttacatctagcaacaagtccacttgcccgctctgccgtgagacctttttctgaggtttttttcattggaagttgtcgctgccgtaggtcaagccaaagggaatggattggctccaccttgaagtactgatgtgaagccagtgagcatgacaaagtgccatcg

SEQ ID NO: 2 - DNA, 5444 bp, Homo sapiens
cagcaaatggacaggtgggtggcggaaaagggcccgggggaagttattacagggtgtcctcttccgccgccagaagccggaagttgtgtcccggacgtgtcaaccggggtctgagtgctcagagtacagctgcaaccgcgaccatgggcgggaagaacaagcagcgaactaaagggaacctgaggccttcaaacagtggccgagctgcagaactccttgccaaagaacagggaacagtgcctggatttattggttttggaacatctcagagtgacctaggctatgttcctgctattcaaggagctgaagaaattgacagtcttgtagattctgatttccgaatggtgctgcggaaactttcaaagaaagatgtcaccacaaaattaaaagctatgcaggaatttggaaccatgtgtacagagagagacacagaaactgtgaaaggagttcttccatattggccaagaattttttgcaaaatttcacttgatcatgaccgtcgcgtccgagaagccacacaacaagcttttgaaaaacttatccttaaagtaaagaaacagttggctccctacttaaaaagtttaatgggatattggctaatggctcagtgtgatacttacacaccagctgcgtttgcagcaaaagatgcatttgaagcggcttttcctccaagcaagcaacctgaagccatagcattttgtaaggatgaaattacaagtgtgctgcaggatcatcttataaaagaaacacctgatacactcagtgacccgcaaactgttccagaggaagaaagagaagctaaattctaccgggttgtaacttgttccttattggcattaaagagattactttgccttttacctgataatgagcttgattctctggaggagaaatttaagtctcttttatcacagaataagttttggaagtatggaaaacacagtgtacctcagattcgctcagcttattttgagttagtctctgcattgtgccagcgcattccacagttgatgaaagaggaagcatccaaagtgagcccatcagttctacttagcattgatgacagtgacccaattgtctgcccagctctctgggaagctgtactctatacacttacaactattgaggactgttggcttcatgtaaatgcaaaaaagagtgtgtttcccaagctatcaactgtgattcgtgaaggtggtcggggtctagctactgtcatatatccttaccttctgccattcatcagcaagctccctcagtccatcacaaatccaaagttggatttcttcaaaaatttcctcacgtctctagttgctgggctgtcaacagagagaactaaaaccagctctttagagtcctcggcagtaatatctgctttttttgaatgcttacgttttataatgcagcaaaacttaggtgaggaagagattgaacagatgctcgtcaatgatcagttgatcccttttattgatgcagttctcaaagacccaggattgcaacatgggcagctatttaaccatttagcagaaactctaagttcctgggaagccaaagcagacacggaaaaagatgaaaaaacagctcacaacttggagaacgtactgatacatttctgggaaagactgtcagagatctgtgttgcgaaaatcagtgagccagaagctgatgttgagtccgttttgggtgtatctaacctattacaggtgcttcagaagccgaagagctcattgaagtcaagtaaaaaaaaaaatggtaaggttagatttgctgatgagatacttgaaagcaataaagagaatgaaaaatgtgtatcttcagaaggagagaagattgaaggctgggaattaacaactgaaccttctctcactcataattcttcaggccttttgtctcctctaaggaaaaaacctttggaagacttagtctgtaaactcgcagatataagtattaattatgtcaatgaacgaaagtcagagcaacatctaaggtttctttctactctgcttgactccttttcttcaagccgagtatttaaaatgctacttggtgatgaaaaacagagtattgtccaagccaaacctcttgaaatagccaagcttgtacaaaaaaatcctgcggtgcagtttttataccagaaactgataggttggctaaatgaagatcaaaggaaggattttggtttcctggtggacattttgtacagtgctctccggtgctgtgacaatgatatggaaagaaaaaaagtcttggatgatctaaccaaggtggacttgaaatggaattctcttcttaagattattgaaaaggcatgtcctagttcagataaacatgctttagtaactccttggctcaaaggcgatatccttggtgagaaattggtcaacttggcagattgtctttgtaatgaggacttggaatccagggtatcttcagaatctcacttctcagaaagatggactcttctaagcttggtattatcccaacatgttaaaaatgattacttgattggagacgtatatgttgaaagaatcattgttagacttcatgaaactttattcaaaacaaagaaattatcagaagctgaaagcagtgactcatcagtgtcttttatctgtgatgtggcctataactatttcagctcagcgaaaggatgcttgctaatgccatcatctgaagatttattattaactctctttcagttatgtgctcagagcaaagaaaaaacacatttgccagattttcttatctgtaaactgaaaaatacttggctctctggtgtaaatttattggttcatcaaactgacagttcatataaagagagtaccttcctacatttgtctgctctgtggctgaagaaccaagttcaggcttcatctttggatatcaacagtctccaagtcctcttgtctgctgttgatgatttgctaaatacacttctagagagtgaagattcttatcttatgggagtttatattggaagtgtaatgccgaacgacagtgaatgggaaaagatgaggcagtctcttcctatgcagtggttacatagacctcttttagagggaagattgagtttgaattatgaatgtttcaaaacagattttaaggaacaggacataaagacacttcccagccatttgtgtacttcagcattattgagcaaaatggtcttaattgcactgagaaaggaaacagtcttagaaaataatgagcttgagaaaataattgcagaactgctttattcactgcagtggtgtgaagaattagataacccacctatttttctaattggattttgtgaaatacttcaaaaaatgaatattacgtatgataacttacgtgtacttggtaatacgtcgggccttttgcagctgttatttaacaggtccagagaacatggcacactgtggtctcttattattgctaagttgatcctttcccgaagcatttcatctgatgaagtaaaaccacattataagagaaaagaaagtttttttccactaactgaaggcaatttgcataccattcaaagtctatgtccatttttgtcaaaagaagaaaagaaagaatttagtgctcaatgtatacctgctcttttgggctggactaagaaagatctttgcagcactaatggaggttttggacatcttgccattttcaattcttgtctgcaaaccaaaagtatagatgatggagagctattacatggaatattaaaaatcataatatcctggaagaaagagcatgaagatatttttcttttcagttgtaatctatcagaagcaagtccagaggtactgggtgtaaatatagaaataatccggtttctttccctatttctgaaatactgctcatcccctttggcagagagtgagtgggacttcatcatgtgctccatgttggcttggttggagacaacaagtgagaatcaggcattgtattctattccacttgtgcaactgtttgcctgtgtcagctgtgatttggcctgtgacctcagtgctttctttgattccacaactctggataccattggcaatcttcctgtaaatctaatcagtgaatggaaagaatttttttcccaaggcatccacagtttgcttttacctattttggtgactgttacaggagaaaacaaagatgtgtctgaaacatcctttcagaatgcaatgctgaaacccatgtgtgaaacattaacgtatatctcaaaggaacagctattgagtcacaaacttcctgcaagattagttgctgaccaaaaaacaaacttaccagaatatctccagactttgttaaatacattggccccattactcctcttcagagctaggcctgtgcaaattgctgtttatcatatgctatacaaattgatgcctgaattaccacagtatgatcaggataatctaaagtcatacggagatgaagaagaagagccagccttgtcaccaccagcagcactgatgtctcttcttagcattcaagaggacttactagaaaatgttttggggtgtattcctgttggacagatagttactattaaaccactgagtgaagacttctgttatgttctgggataccttctcacttggaaattaatactaactttcttcaaagctgcatcatcacagcttcgggctttgtattccatgtatcttcggaaaacaaagagtttgaataaattgctctatcacctgttcaggcttatgccagaaaatccaacctatgcagagacagcagttgaggtcccaaataaggaccctaaaacattctttactgaggagctccagctgagtattagagaaacaacaatgcttccataccacattccacacttggcttgttcagtctatcatatgacattaaaagacttgcctgccatggttaggttgtggtggaatagcagtgagaagcgtgttttcaatattgtggatagatttacaagcaagtatgtcagcagtgttctttcttttcaagaaatatcttctgtacaaacaagtacacaactatttaatggcatgacggttaaagctcgagctactactcgagaggtaatggctacttatactattgaggacatagttattgaacttataatacaactgccttcaaattatccactgggttcaataatagtagaaagtgggaaaagagtaggagtagctgttcagcagtggcggaactggatgctgcagttaagcacttacctcacccatcagaatggaagtattatggaaggcttagctttatggaaaaataacgtagacaaacgttttgagggtgttgaagattgcatgatctgtttctcagtcattcacggtttcaactattcccttcccaaaaaagcctgtagaacatgcaagaaaaaattccattcagcctgcttgtacaaatggtttacatctagcaacaaatccacttgtccactgtgtcgtgagacgtttttctga

SEQ ID NO: 3 - protein, 14 aa, Mus musculus
KEDLKWSSLLQVIE

SEQ ID NO: 4 - protein, 14 aa, Homo sapiens
KVDLKWNSLLKIIE

SEQ ID NO: 5 - protein, 1753 aa, Mus musculus
MGGKNKQRTKGNLRPSNSGRAAELLAKEQGTVPGFIGFGTSHSDLGYVPAVQGAEDIDSLVDSDFRMVLRKLSKKDVTTKLKAMQEFGIMCTERDTEAVKGVLPYWPRIFCKISLDHDRRVREATQQAFEKLILKVKKHLAPYLKSVMGYWLMAQCDTYPPAALAAKDAFEAAFPPSKQPEAIAFCKEEITTVLQDHLLKETPDTLSDPQTVPEEEREAKFHRVVTCSLLALKRLLCFLPNNELDSLEEKFKSLLSQNKFWKYGKHSVPQVRSAYFELVSALCQHVPQVMKEEAAKVSPSVLLSIDDSDPVVCPALWEAVLYTLTTIEDCWFHVNAKKSVFPKLMAMIREGGRGLAAVMYPYLLPFISKLPQSITEPKLDFFKNFLTSLVTGLSTERTKSSSSECSAVIPAFFECLRFIMQQNLGEEEMVQMLINEQLIPFIDTVLKDSGLHHGPMFDHLADTLSSWEAKADAERDPGAVYNLENVLLSFWGRLSEICTEKIRQPEADVKSVLCVSSLVGVLQRPRSSLELHRKKTAQVRFAINIPEAHKGDEKSMSSEGENSEGSDGGAQSPLSNTSSDLVSPLRKKPLEDLVCKLAEVSISFVNERKSEQHLQFLSTLLDSFSSVQVFNILLSDKQKNVVKAKPLEITKLAEKNPAVKFLYHKLIGWLNDSQKEDGGFLVDILYSALRCCDSGVERKEVLDDLTKACSSSDKHALVTPWLKGSILGEKLVALADCLCDKDLEATTSESHSSEQWSLLRLALSQHVKNDYLIGEVYVGRIIVKLHETLSKTKDLSEAANSDSSVSFVCDVVHSFFSSAGGGLLMPPSEDLLLTLFQLCAQSKERTHLPDFLICKLKNTLLSGVNLLVHQTASTYEQSTFLRLSVLWLKDQVQSSALDNTSLQVLLSAAGDLLGTLVESEDTSLLGVYIGSVMPSDSEWEKMRQALPVQWLHRPLLEGRLSLNYECFKTDFKEQDTKTLPNHLCTSSLLSKMILVAQKKKLVLEDNVLEKIIAELLYSLQWCEELDNAPSFLSGFCGILQKMNITYSNLSVLSETSSLLQLLFDRSRKNGTLWSLIIAKLILSRSISSDEVKPYYKRKESFFPLTEGSLHTIQSLCPFLSKEEKKEFSAHSIPAFLGWTKEDLCSINGAFGHLAIFNSCLQTRSIDDKQLLHGILKIITSWRKQHEDIFLFSCNLSEASPEVLGLNIEIMRFLSLFLKHCAYPLPLADSEWDFIMCSMLAWLETTSENQALYSVPLVQLFACVSFDLACDLCAFFDSITPDIVDNLPVNLISEWKEFFSKGIHSLLLPLLVNAIGENKDLSETSFQNAMLKPMCETLTYISKDQLLSHKLPARLVASQKTNLPEHLQTLLNTLTPLLLFRARPVQIAAYHMLCKLMPELPQHDQDNLRSYGDEEEEPALSPPAALMSLLSSQEELLENVLGCVPVGQIVTVKPLSEDFCYVLGYLLTWKLILTFFKAASSQLRALYSMYLRKTKSLNKLLYHLFRLMPENPTYGETAIEVSSKDPKTFFTEEVQLSIRETATLPYHIPHLACSVYHMTLKDLPAMVRLWWNSSEKRVFNIVDRFTSKYVSNVLSFQEISSVQTSTQLFNGMTVKARATTREVMATYTIEDIVIELIIQLPSNYPLGSITVESGKRIGVAVQQWRNWMLQLSTYLTHQNGSIMEGLALWKNNVDKRFEGVEDCMICFSVIHGFNYSLPKKACRTCKKKFHSACLYKWFTSSNKSTCPLCRETFF

SEQ ID NO: 6 - protein, 1752 aa, Homo sapiens
MGGKNKQRTKGNLRPSNSGRAAELLAKEQGTVPGFIGFGTSQSDLGYVPAIQGAEEIDSLVDSDFRMVLRKLSKKDVTTKLKAMQEFGTMCTERDTETVKGVLPYWPRIFCKISLDHDRRVREATQQAFEKLILKVKKQLAPYLKSLMGYWLMAQCDTYTPAAFAAKDAFEAAFPPSKQPEAIAFCKDEITSVLQDHLIKETPDTLSDPQTVPEEEREAKFYRVVTCSLLALKRLLCLLPDNELDSLEEKFKSLLSQNKFWKYGKHSVPQIRSAYFELVSALCQRIPQLMKEEASKVSPSVLLSIDDSDPIVCPALWEAVLYTLTTIEDCWLHVNAKKSVFPKLSTVIREGGRGLATVIYPYLLPFISKLPQSITNPKLDFFKNFLTSLVAGLSTERTKTSSLESSAVISAFFECLRFIMQQNLGEEEIEQMLVNDQLIPFIDAVLKDPGLQHGQLFNHLAETLSSWEAKADTEKDEKTAHNLENVLIHFWERLSEICVAKISEPEADVESVLGVSNLLQVLQKPKSSLKSSKKKNGKVRFADEILESNKENEKCVSSEGEKIEGWELTTEPSLTHNSSGLLSPLRKKPLEDLVCKLADISINYVNERKSEQHLRFLSTLLDSFSSSRVFKMLLGDEKQSIVQAKPLEIAKLVQKNPAVQFLYQKLIGWLNEDQRKDFGFLVDILYSALRCCDNDMERKKVLDDLTKACPSSDKHALVTPWLKGDILGEKLVNLADCLCNEDLESRVSSESHFSERWTLLSLVLSQHVKNDYLIGDVYVERIIVRLHETLFKTKKLSEAESSDSSVSFICDVAYNYFSSAKGCLLMPSSEDLLLTLFQLCAQSKEKTHLPDFLICKLKNTWLSGVNLLVHQTDSSYKESTFLHLSALWLKNQVQASSLDINSLQVLLSAVDDLLNTLLESEDSYLMGVYIGSVMPNDSEWEKMRQSLPMQWLHRPLLEGRLSLNYECFKTDFKEQDIKTLPSHLCTSALLSKMVLIALRKETVLENNELEKIIAELLYSLQWCEELDNPPIFLIGFCEILQKMNITYDNLRVLGNTSGLLQLLFNRSREHGTLWSLIIAKLILSRSISSDEVKPHYKRKESFFPLTEGNLHTIQSLCPFLSKEEKKEFSAQCIPALLGWTKKDLCSTNGGFGHLAIFNSCLQTKSIDDGELLHGILKIIISWKKEHEDIFLFSCNLSEASPEVLGVNIEIIRFLSLFLKYCSSPLAESEWDFIMCSMLAWLETTSENQALYSIPLVQLFACVSCDLACDLSAFFDSTTLDTIGNLPVNLISEWKEFFSQGIHSLLLPILVTVTGENKDVSETSFQNAMLKPMCETLTYISKEQLLSHKLPARLVADQKTNLPEYLQTLLNTLAPLLLFRARPVQIAVYHMLYKLMPELPQYDQDNLKSYGDEEEEPALSPPAALMSLLSIQEDLLENVLGCIPVGQIVTIKPLSEDFCYVLGYLLTWKLILTFFKAASSQLRALYSMYLRKTKSLNKLLYHLFRLMPENPTYAETAVEVPNKDPKTFFTEELQLSIRETTMLPYHIPHLACSVYHMTLKDLPAMVRLWWNSSEKRVFNIVDRFTSKYVSSVLSFQEISSVQTSTQLFNGMTVKARATTREVMATYTIEDIVIELIIQLPSNYPLGSIIVESGKRVGVAVQQWRNWMLQLSTYLTHQNGSIMEGLALWKNNVDKRFEGVEDCMICFSVIHGFNYSLPKKACRTCKKKFHSACLYKWFTSSNKSTCPLCRETFF

SEQ ID NO: 7 - protein, 1767 aa, Mus musculus
MGGKNKQRTKGNLRPSNSGRAAELLAKEQGTVPGFIGFGTSHSDLGYVPAVQGAEDIDSLVDSDFRMVLRKLSKKDVTTKLKAMQEFGIMCTERDTEAVKGVLPYWPRIFCKISLDHDRRVREATQQAFEKLILKVKKHLAPYLKSVMGYWLMAQCDTYPPAALAAKDAFEAAFPPSKQPEAIAFCKEEITTVLQDHLLKETPDTLSDPQTVPEEEREAKFHRVVTCSLLALKRLLCFLPNNELDSLEEKFKSLLSQNKFWKYGKHSVPQVRSAYFELVSALCQHVPQVMKEEAAKVSPSVLLSIDDSDPVVCPALWEAVLYTLTTIEDCWFHVNAKKSVFPKLMAMIREGGRGLAAVMYPYLLPFISKLPQSITEPKLDFFKNFLTSLVTGLSTERTKSSSSECSAVIPAFFECLRFIMQQNLGEEEMVQMLINEQLIPFIDTVLKDSGLHHGPMFDHLADTLSSWEAKADAERDPGAVYNLENVLLSFWGRLSEICTEKIRQPEADVKSVLCVSSLVGVLQRPRSSLELHRKKTAQVRFAINIPEAHKGDEKSMSSEGENSEGSDGGAQSPLSNTSSDLVSPLRKKPLEDLVCKLAEVSISFVNERKSEQHLQFLSTLLDSFSSVQVFNILLSDKQKNVVKAKPLEITKLAEKNPAVKFLYHKLIGWLNDSQKEDGGFLVDILYSALRCCDSGVERKEVLDDLTKEDLKWSSLLQVIEKACSSSDKHALVTPWLKGSILGEKLVALADCLCDKDLEATTSESHSSEQWSLLRLALSQHVKNDYLIGEVYVGRIIVKLHETLSKTKDLSEAANSDSSVSFVCDVVHSFFSSAGGGLLMPPSEDLLLTLFQLCAQSKERTHLPDFLICKLKNTLLSGVNLLVHQTASTYEQSTFLRLSVLWLKDQVQSSALDNTSLQVLLSAAGDLLGTLVESEDTSLLGVYIGSVMPSDSEWEKMRQALPVQWLHRPLLEGRLSLNYECFKTDFKEQDTKTLPNHLCTSSLLSKMILVAQKKKLVLEDNVLEKIIAELLYSLQWCEELDNAPSFLSGFCGILQKMNITYSNLSVLSETSSLLQLLFDRSRKNGTLWSLIIAKLILSRSISSDEVKPYYKRKESFFPLTEGSLHTIQSLCPFLSKEEKKEFSAHSIPAFLGWTKEDLCSINGAFGHLAIFNSCLQTRSIDDKQLLHGILKIITSWRKQHEDIFLFSCNLSEASPEVLGLNIEIMRFLSLFLKHCAYPLPLADSEWDFIMCSMLAWLETTSENQALYSVPLVQLFACVSFDLACDLCAFFDSITPDIVDNLPVNLISEWKEFFSKGIHSLLLPLLVNAIGENKDLSETSFQNAMLKPMCETLTYISKDQLLSHKLPARLVASQKTNLPEHLQTLLNTLTPLLLFRARPVQIAAYHMLCKLMPELPQHDQDNLRSYGDEEEEPALSPPAALMSLLSSQEELLENVLGCVPVGQIVTVKPLSEDFCYVLGYLLTWKLILTFFKAASSQLRALYSMYLRKTKSLNKLLYHLFRLMPENPTYGETAIEVSSKDPKTFFTEEVQLSIRETATLPYHIPHLACSVYHMTLKDLPAMVRLWWNSSEKRVFNIVDRFTSKYVSNVLSFQEISSVQTSTQLFNGMTVKARATTREVMATYTIEDIVIELIIQLPSNYPLGSITVESGKRIGVAVQQWRNWMLQLSTYLTHQNGSIMEGLALWKNNVDKRFEGVEDCMICFSVIHGFNYSLPKKACRTCKKKFHSACLYKWFTSSNKSTCPLCRETFF

SEQ ID NO: 8 - protein, 1766 aa, Homo sapiens
MGGKNKQRTKGNLRPSNSGRAAELLAKEQGTVPGFIGFGTSQSDLGYVPAIQGAEEIDSLVDSDFRMVLRKLSKKDVTTKLKAMQEFGTMCTERDTETVKGVLPYWPRIFCKISLDHDRRVREATQQAFEKLILKVKKQLAPYLKSLMGYWLMAQCDTYTPAAFAAKDAFEAAFPPSKQPEAIAFCKDEITSVLQDHLIKETPDTLSDPQTVPEEEREAKFYRVVTCSLLALKRLLCLLPDNELDSLEEKFKSLLSQNKFWKYGKHSVPQIRSAYFELVSALCQRIPQLMKEEASKVSPSVLLSIDDSDPIVCPALWEAVLYTLTTIEDCWLHVNAKKSVFPKLSTVIREGGRGLATVIYPYLLPFISKLPQSITNPKLDFFKNFLTSLVAGLSTERTKTSSLESSAVISAFFECLRFIMQQNLGEEEIEQMLVNDQLIPFIDAVLKDPGLQHGQLFNHLAETLSSWEAKADTEKDEKTAHNLENVLIHFWERLSEICVAKISEPEADVESVLGVSNLLQVLQKPKSSLKSSKKKNGKVRFADEILESNKENEKCVSSEGEKIEGWELTTEPSLTHNSSGLLSPLRKKPLEDLVCKLADISINYVNERKSEQHLRFLSTLLDSFSSSRVFKMLLGDEKQSIVQAKPLEIAKLVQKNPAVQFLYQKLIGWLNEDQRKDFGFLVDILYSALRCCDNDMERKKVLDDLTKVDLKWNSLLKIIEKACPSSDKHALVTPWLKGDILGEKLVNLADCLCNEDLESRVSSESHFSERWTLLSLVLSQHVKNDYLIGDVYVERIIVRLHETLFKTKKLSEAESSDSSVSFICDVAYNYFSSAKGCLLMPSSEDLLLTLFQLCAQSKEKTHLPDFLICKLKNTWLSGVNLLVHQTDSSYKESTFLHLSALWLKNQVQASSLDINSLQVLLSAVDDLLNTLLESEDSYLMGVYIGSVMPNDSEWEKMRQSLPMQWLHRPLLEGRLSLNYECFKTDFKEQDIKTLPSHLCTSALLSKMVLIALRKETVLENNELEKIIAELLYSLQWCEELDNPPIFLIGFCEILQKMNITYDNLRVLGNTSGLLQLLFNRSREHGTLWSLIIAKLILSRSISSDEVKPHYKRKESFFPLTEGNLHTIQSLCPFLSKEEKKEFSAQCIPALLGWTKKDLCSTNGGFGHLAIFNSCLQTKSIDDGELLHGILKIIISWKKEHEDIFLFSCNLSEASPEVLGVNIEIIRFLSLFLKYCSSPLAESEWDFIMCSMLAWLETTSENQALYSIPLVQLFACVSCDLACDLSAFFDSTTLDTIGNLPVNLISEWKEFFSQGIHSLLLPILVTVTGENKDVSETSFQNAMLKPMCETLTYISKEQLLSHKLPARLVADQKTNLPEYLQTLLNTLAPLLLFRARPVQIAVYHMLYKLMPELPQYDQDNLKSYGDEEEEPALSPPAALMSLLSIQEDLLENVLGCIPVGQIVTIKPLSEDFCYVLGYLLTWKLILTFFKAASSQLRALYSMYLRKTKSLNKLLYHLFRLMPENPTYAETAVEVPNKDPKTFFTEELQLSIRETTMLPYHIPHLACSVYHMTLKDLPAMVRLWWNSSEKRVFNIVDRFTSKYVSSVLSFQEISSVQTSTQLFNGMTVKARATTREVMATYTIEDIVIELIIQLPSNYPLGSIIVESGKRVGVAVQQWRNWMLQLSTYLTHQNGSIMEGLALWKNNVDKRFEGVEDCMICFSVIHGFNYSLPKKACRTCKKKFHSACLYKWFTSSNKSTCPLCRETFF

SEQ ID NO: 9 - protein, 1747 aa, Drosophila melanogaster
MGGKTKQAPRTKNNAKPSSSSRTAELLGSSTPIFVGFSAQTDGGGLVPFAPGFASAEQMPDSFDAAISPQTQIILRKLSKKDPMTKKKALQELHELIEQSDVEVLKNILPLWPKYYLNLASDPEHTVREQTQTVLQLLMAKCKKAMAPYLKLLVPVWLGSRFDTYAPAASIASQSFRDTFAGNANRSREVCMHCQVEILEYATRNLTFHTAATLSIGKSLTPEDAEQKYQRVIISSLKLLSFFMGQTAQTEELSQVKEGFGTLVAHQKFWSFAKHKVPAIKAAWFECIYHILQSVALLDVITPQKTQLTNLCFQFIDDADPVVAPHIWGCVLLLQSNYVDWFVPLNIRKTLLPKLSSLLQNGFNRNAQAICPNLLPFLSKVTQASLQDLDIYDFYQRFFDDMKLAVTKKFDPPLSKSDCIVIHNAYFECLRFLMQQINNNKQREQKEEEFSFSLLDNNVLEPIAWLLKSDSTHVKIFFQHSSALVAFWDRQINNRLDNGDLYAKLLNKFWIRIFELVTQDLSAEEVNEQLLGHVLLLVQDLHMANPSLESPSVKFVEGPNEKIEKSEPTTPVKKAQEAAAFIQKELKQLVIKLVRICLDKANKGSGSGTSSSRYIEQIRTLTKMFNDAAFYKSLTDDGDLASALNKFVSLLGQLSCQACESVVEIVFEILPLLETGKRFEYIENTLMKLPQHGVQNLLLHRLLSYPLCAEAAVRQMLSGPETCEMIARIAEEVVVDNDREKLNLLHKCFFQTDTGDILINAKTVDKILLSMCGPLEQPVVDDAVEVCGSFIAQIMPVICSNNNSSLHVRQHIFLKLFKFSLEHRPEDYLSEDTLWEITTCWQDGLSSKDIEIDDDMLKCCAGIVEELANSAELKADTLDGMAEAMAKFVICSTENIEDEYKRLERIDETLTALLETPLKTTDKVQQFENHCVLLEALHGSVTAGVPFENACLSRNEILPLLQRSTLNFSTIYKLVYQFPPPQDTNDPEDELTEDYCDPNADVLKKWNEPLIAELLQCIRVAGTAECWLEMSVLQSSTEELVLILSEKVQSFMGNSSDLVAIVKERLQQAAVQQSSVIDCRLLSYLRFCPQYAAFEESASILLHEDLSENLVTQGALKTYVIALQFLLPKLSQKAITLSSAIMGTEPPEIWVKAAVFHALLLNNFEGDVNEQTDRNIIVSAVQFMTSIGERQASQKDLLHYNVEIQRQPYESVINTVEFIKLLTEVLKRFPYELSIKNWDAIRIGLSSWVLSVSKSIAQYQDPKTSLFIVAVYELFAALIDFIRSEKQKSSTELLKNMIDEWDSLFAKEVNLVLFKSYYLLTHEVSVDPGFQACYEALLEQITPVIERLDYSFVYSFCKSNSNITLDHLCNFLFKQLYSVQHSVRLSAVHSLRQLTPHFVADDIELNEKQSESLDASTTICKWHFLNRFEDYLTRYDALITKYLEEFTFKLSELDDLEPIDRHNALSYLFLWDCIINACAKSPVALRAVYTNWLNDNKYEENFLHFLFRAMPVDILKNHGAKVHSNGVYKELTWSQQKDRHLPLERYACHLYTEVLRKLPAVVRRWWNATQSRQKNFIDNLTTNYVSSLICSEELKAIANRKEKHENMQVTVHSSTREVLAVYAIDEARMELVITLAPNYPLGAVKVECGKQIGGRASSRNVGMQLTIFLTHQNGTIYDGLTMWKNNLDKKFEGVEECYVCYTVIHQETCQLPKLTCKTCKKKFHGPCLYKWFTTSSKSTCPICRNVF

SEQ ID NO: 10 - protein, 1566 aa, Caenorhabditis elegans
MKICFFFPPKRAQKWQKKCFFPNVSAEINVASSNRLHQVVEIDDETRIVMKKLTKKDCQTREKGLKELMELINTENSSIESSYEHFCGLVAQLTTDGSPTVRMLTMKVISQFLTKLKKSASKGLKKIIPFVLFAKSDVTNGVAAAASAVIRDGFDADKKRQVVELFVPNTFDLAAKIAEGKHELSLPAEYDASEDLETRKMRLETQSLNTFLSYIKEYGNESKLWEEPARKLFSNSEFIKKTFAGKKEALKVQLLNISYKFSDNIEVILSNPVISTYIQASLDAQTFSTECATAWEGILILLPDERFHAKCSLQKGIYPRLLNLIRKKGNHWRVLKHYLLPAVSVLLQKLENPALITSIITSFTDNLPWQAEASMNAIHCWFCTFSDFVKWILGNDRINLEILKDLSPLIVEMSNQSMHFNTAEATECISGLIHWIIEKKVLENPAEFFDLLKTSIYEVAPPEKSRLFADSLTLPAKHLELAHLHGNLLSNPDVDFHIIRNLARASNSEYFEETCRNINNFEFIENSDRFDMLQAVEIVKLIEMKPSLSLQIKNNHVGRQLLLSENSEIWEKSLKNVPAGVFQEMVNFWHEKRNGKAIAQAVNFLKKMGIQLDTNAAAENVDFLISLLQSLDSKEDPEERKNLVLKLLSALFDAEDEPKLEHFESLKSHLNGDFEQFFEKLFANMEEEDAERVLEIAARFDKLVGFCDADSRGEIAGKMILGRREFDEMSEKLHFLELDVLTVSQHTTIITDALSRPIEHLEEKEATKMVKELGRLALFSVASNYNSSIHQLFAWQMIRVISALGNRYCLKFLDEELQQLRIELEKRVIKSEEIQKLINDGCCCAPNFITDTYGIPEKRQKFEEYSEDMDTKIETIYLKTDTPLEYVEKVFEASQSENSFPLFQFDQSKKYEWLANLTFVKRFIQCGGEIFRAENLEFRDFTLCGIITVLDTSTDILIDSPHSFSENPLLEALTTLYLELFVVLTDATKRGAYSEQSVEEWNEFYTPTIHTYCIRLFRTIRRDQQPTPFVRALLRALFVISEFPTSFSNDDDVANQEFIPELSVFKYPAFQESCIAQAFSLFASNNEHIQLIAYSVARLLMPIMFKLENAAALKSNEDSELPVSTNRRKLSLPVMISKSYPKDHHNPHVGPLLLDLTLLPLENTKDSGFSQEHRVAYCDVIDPFFKNALNALMLDQPFEFRQVPIGCRIQNRKFERKLKKNVKKSEILQFFCRKYGTPGLDAINLCLKKLRTSFYILLKFGFVEISTIFRNMDRKKAEIFKNRKTNNFRSIFHQILFMLMRKKPIKKPRTKGNKCLCFFLININKTRFKNFDFLKKRKIENSKSQERAYYLESDLSASPIFFDKFASRLLFKSMTLLPAAIRLFYKGMPNCFMPMFQETVTKYASRLLIEQELGKVREAKFEGEMKVRTVPVTGEIIAEYVVEETKMKLTIGLPPDYPLSVPSLTLDKAIVKTDRAKKWLLQLNAYLFHQNGAILEGIEMWKRNVDKGVEGVEDCTICMMTVHQQTHQLPKIKCKQCKNKFHSNCLYKWFESSNQSTCPLCRNNFT

SEQ ID NO: 11 - protein, 1562 aa, Saccharomyces cerevisiae
MSFGGINTFQQYNTDLGLGHNGVRISLNYFDGLPDPSLLNSLYSNELKLIFKSLLKRDETTKEKALMDLSNLISDFNQNEYFFNDIFLLCWSQIYAKLIISDYKVIRLQSHQITIMLVKSLRKKISKFLKDFIPLILLGTCELDYSVSKPSLNELTECFNKDPAKINALWAVFQEQLLNLVKEIVVNENEDTISDERYSSKEESEFRYHRVIASAVLLLIKLFVHNKDVSERNSSSLKVILSDESIWKLLNLKNGQNTNAYETVLRLIDVLYTRGYMPSHKNIMKLAVKKLLKSLTHITSKNILKVCPVLPSILNLLATLDDYEDGTIWSYDKSSKEKVLKFLSVSRTSPSPGFFNAVFALYSSTKRHSFLDYYLEWLPFWQKSVQRLNEKGFSARNSAEVLNEFWTNFLKFAEDSSEERVKKMVESEIFNSLSCGKSLSEYTKLNQTLSGVFPPDKWEREIEDYFTSDEDIRKIKVSFEKNLFALLVTSPNNESAISRLFDFFVQLIETDPSNVFNKYDGVYDALNYFLDSDMIFLNGKIGKFINEIPTLVQESTYQNFAGIMAQYSNSKFFKMNTDAITSLEDFFIVALSFNLPKTIILATMNELDNDIYQQLMKSDSLELELYIEDFMKNYKFDDSGEIFKGNNKFLNQRTITTLYRSAVANGQVEQFCAVLSKLDETFFSTLLLNTDFLSCALYEVSEDTNEKLFKLSLQLAKGNSEIANKLAQVILQHAQVYFSPGAKEKYVTHAVELINGCNDTSQIFFPANAIEVFARYMPAIDYRSSLVSSLSTNTHLLLTDDKPINLKNMQKLIRYALFLDALLDALPERVNNHIVAFITVVSELVTDYNCLSEEPNDLYYDFGHTFFKHGKVNLNFSDIVGNVIQPANGGDAMLTFDIAESNSVYFFYYSRVLYKVLLNSIDTVSSTTLNGLLASVESFVTKTVRDQKSTDKDYLLCAILLLMFNRSNSKDEITKLRTLLASQLIGIREVELVDQEFKSLALLNNLLDIPQADKQFVPIAPQRLNMIFRSILKWLDSDLAYEPSFSTVRLLLLDFFTKLMRFEGVRDMGITAFELSERLLADSLSMCQIDDTLYLLELRSSCLNLYETLSQGVSKNGEEISEYGDEIQENLIELMFLNFNQERNNQVSTLFYQKLYKVISSMELKKLESQYKRIFEVVLNDKDIGSNINQSRLLTTLLGSLVVKTQQDIIIEYELRIQKQTGSDVDGSASDNDVNSKFKLPQKLLQKVTDEVPKEYLEYENKNSFIKYLWYWHLILMYFKDTSYNMRQIFIEQLKEAGLINRMFDFITDQIDLRDTEFWKQVDTKEISEYNIVGNNFSPYKEDIFEECKKLLGHTLYQLFNNVGCLTSIWWLNIKDRTLQNDIEKFVSEFISPILIKNEFDDINSKMDRLTSNDDALTIKLNNITNEVKASYLIDDQKLEISFKLPKNYPLTNIQVNGVSRVGISEQKWKQWIMSTQHVITGMNGSVLDSLELFTKNVHLQFSGFEECAICYSILHAVDRKLPSKTCPTCKNKFHGACLYKWFRSSGNNTCPLCRSEIPFRR

SEQ ID NO: 12 - DNA, 183 bp, Mus musculus
gaatccccttccggcgcgagggaccggaagttgtggcccgcccgtgtcaacttcggctctgcagactgagagccgggctgccaccgccaccatgggcgggaagaacaagcagcggactaaggggaacctcagggtgagtcctgtggtgatggtagccccgcggcctgcggcccgggtgtccag

SEQ ID NO: 13 - DNA, 308 bp, Mus musculus
agaaagtgttctaacttttttgtttttgctttgttttgctttcttatcagccttcaaacagtggccgagctgcagaactcctcgccaaagagcagggaacagtgcctgggttcattggttttggcacatctcacagtgacctgggctatgttccggctgttcaaggcgcggaagacatagacagtcttgtagattccgatttccgaatggtgctgcggaaactttccaaaaaagatgttacaacaaagctaaaggcaagttttctcatttttacaatagtaattaagaatgtagttttgttagaataa

SEQ ID NO: 14 - DNA, 195 bp, Mus musculus
ggtcgtgttacctctgttaacccttgatgatgttaattattttcaggcaatgcaagaatttggaattatgtgcacagagagagacacagaagccgtcaaaggggttcttccatactggccaagaatcttctgcaaaatctcccttgtaagcaagcattagcgttcatgagctgtttctgcttaaagggctacctc

SEQ ID NO: 15 - DNA, 331 bp, Mus musculus
tcttacaaatattagtatttgttcttaaaccacatttctgctttcaataggatcatgatcgccgtgttcgagaggcgacgcagcaagcttttgaaaaacttatccttaaagtaaagaagcacttagctccctatttaaaaagcgtgatgggctattggttgatggctcagtgtgacacatatccaccagctgcactggcagcaaaagatgcattcgaagccgctttccctccaagcaagcaacctgaagccatagcgttttgcaaggaagaaattacaactgtgagtgtttgcagccactgtgtccccgtgtttctcagttgtctaaggct

SEQ ID NO: 16 - DNA, 153 bp, Mus musculus
atggttgtcagtgccataagtctcaaggctttggtggttctctccttcaggtgttacaggaccatcttctgaaggagactcctgacacactcagtgaccctcagtaagctgcatgtttctaactaatgtgcactaggcatttacttagttggt

SEQ ID NO: 17 - DNA, 281 bp, Mus musculus
tgtagtttggcttcttttttgcagtcttacttgtttatttttaactctagaactgtgccagaggaagagagagaggctaagttccaccgagtcgtgacgtgctctttattggcactgaagagattgctttgtttcttacctaacaatgagcttgattctctggaggagaaatttaaatcacttttatcacagaataaattttggaagtatggaaaacacagtgtacctcaggtatgccaatagcttcgagtttaaaacttgttttctgtaactccgccacg

SEQ ID NO: 18 - DNA, 274 bp, Mus musculus
ctactgtctttaggctttggtttcatcccgctttctggttcttcttttaggtccgctcagcatattttgagttagtttctgctttgtgccagcacgttccccaggtgatgaaagaggaagctgccaaagtgagtccatctgtcctgctcagcattgatgacagtgaccctgtggtctgcccagctctctgggaggctgtgctctacacgctgacaactattgaggtatgtaagggagacacacttccctaaacgccacgtgaaagagtgcatta

SEQ ID NO: 19 - DNA, 291 bp, Mus musculus
cacttacaatttttaattactattataaattttttttctcttttggttaggactgttggtttcatgtaaatgccaagaaaagtgtgtttccgaagctgatggccatgatccgagagggtggccggggcctagctgctgttatgtatccttacctcttaccgttcatcagcaaactccctcagtccatcacagagccaaagctggacttcttcaaaaacttcctcacctctctagttaccgggtgagcaaatacatttctttttactttaaaaattacgtttattgtttatt

SEQ ID NO: 20 - DNA, 236 bp, Mus musculus
gagctcttcttttcctcttggaagaataaccctcagtcactacttcctaggctgtcaactgagaggaccaaatcgagctcttctgagtgctcagctgtcatcccggcattttttgaatgtttgcggtttataatgcagcagaacttaggcgaggaggagatggtgcagatgcttatcaatgagcaggtacgtttgctcagcatccttatcaatgagcaggtacgtttgctcagcat

SEQ ID NO: 21 - DNA, 910 bp, Mus musculus
ttaaactcttcttgaagactaaaatttaaattttgtgccttctctttcagttgatcccatttattgatacagtcctcaaagactcaggactgcaccacggaccgatgtttgaccatttagcagacacattgagctcttgggaagccaaagcagatgcagagcgagaccctggagctgtttacaacttggagaacgttctgctaagcttctggggaagactgtcagagatctgcaccgagaaaatccgccagccagaagcagatgtgaagtccgttctgtgtgtgtctagcctagtaggggtccttcagaggccaaggagctcactggagttacacagaaagaaaactgctcaggtcaggtttgccatcaatatacctgaagctcataaaggggatgaaaagtccatgtcttcagaaggagagaacagcgagggctctgacggaggggctcagtctcctctcagtaatacatcttcagacttagtatctcctttaaggaaaaaacctctggaagacttagtctgtaagctggcagaggtgagcattagctttgtcaacgagcggaagtcagagcagcacctgcagtttctctccacgctgctcgactccttctcctccgtccaagtatttaacattctcctcagtgacaaacagaagaatgtcgtcaaagccaaacctctggaaataaccaaacttgcagaaaaaaatcctgcagttaagttcttatatcataaattgataggctggctaaatgacagccagaaagaggacgggggcttcctggtggacatcttgtatagtgccctccgatgctgtgacagtggtgtggaaaggaaagaagtcttggatgatctaaccaaggtacggctgtcatctgactgtcataaacatccttgttgtgtgtggtaagg

SEQ ID NO: 22 - DNA, 142 bp, Mus musculus
tttttaaagctgtatttttgtccttaactctccaactttgtctgtttcaggaggacctgaagtggagttctcttcttcaggtcattgaaaaggtattttagccccctctcccctttccctttctttttcaatttatgtgtgt

SEQ ID NO: 23 - DNA, 287 bp, Mus musculus
tagacacatcttttgatgttgcttttaatctttaattttatggtttttaggcatgttctagctcagataaacatgctttagtaactccttggctaaaaggcagtattcttggagagaaattggttgccttggcagactgtctttgtgataaggacttggaagccacaacatctgaatcccactcatcagaacagtggagtctgctaagactggcgttatcccaacatgtcaaaaacggtagggaatacaactccagctttatagcagggttgataatcctttccacc

SEQ ID NO: 24 - DNA, 340 bp, Mus musculus
aagatattttcaaaatacattgttaatactctttacatttttttttgtagattacttgattggagaagtatacgttggaagaattattgttaaacttcatgaaactttatctaaaacaaaggatttatcagaagcggcaaacagtgactcttcagtgtcttttgtctgtgatgtggtccatagcttcttcagttcagcaggaggaggcttgctaatgccaccatctgaagacttgttattaactctctttcagttatgcgctcagagcaaagaacggacacacttgccaggtaagagcctgctgctcagatattttctaggcaatctcggctctgacctc

SEQ ID NO: 25 - DNA, 254 bp, Mus musculus
agtctatttatagtgagattacaaattatcgctaataccttgatttttagattttctgatctgtaaactgaagaatactttgctttctggtgtaaatttattggtccatcaaactgcaagtacatatgagcagagtaccttcctacgtttgtctgttctgtggctgaaggaccaggttcagtcttcggctttggataacacaaggtaacattttgtgcacagttacatggtgtcccttatatactgtctccttc

SEQ ID NO: 26 - DNA, 245 bp, Mus musculus
tatggaattgcttgtaccctaactacagcgcttctttccattgtttttagtcttcaggtcctcttgtctgctgctggcgacttgctgggtactcttgtagagagtgaggacacgtctcttcttggagtttatatcggaagtgtaatgcccagtgacagcgagtgggaaaagatgaggcaggctcttcctgttcaggtgtgtcttccactgcagcgcgcagctcatcctggatttctagtccattc

SEQ ID NO: 27 - DNA, 287 bp, Mus musculus
tttagactatcgaatgaattttcatacaattgagtgtgttctatttacagtggttacacagacctcttttagagggaaggctgagtttaaattatgaatgcttcaagacagattttaaggaacaagacacaaagactcttcccaaccatctgtgtacttcatcgttattgagcaaaatgatcttagttgcccaaaaaaagaaattagtcctagaagacaacgtccttgaaaaaataagtaagtatctgcgaggttcaggtagacactctgtgatcttcttctgtgct

SEQ ID NO: 28 - DNA, 260 bp, Mus musculus
ggttctgtacagtgtgtgcctttgataactggctgtgtccattctcttagttgcagagctgctgtattcactgcagtggtgtgaagaactcgacaatgcgccatctttcctaagtggattttgtggaatccttcaaaaaatgaatataacttacagcaacttatctgtacttagtgagacttctagcctcttgcagctgttatttgacaggtaagacgccctttcaatttgttttaaaattggctttcccatccataaga

SEQ ID NO: 29 - DNA, 205 bp, Mus musculus
ctctatctgtatagctatatttaacattcatgttttgcttctttttttagatctagaaaaaatggcacactttggtctcttattattgctaagttgatcctttcccgaagtatttcatctgatgaagtgaaaccatactataaaagaaaagaaaggtacccttagttactatgtttccactctgcagtttattattagagtgggt

SEQ ID NO: 30 - DNA, 243 bp, Mus musculus
catcattttgcacatagaataactgtaagtaatgttttctctcttaacagtttcttccctctaacagaaggtagtttgcataccattcaaagtctctgtccattcttgtctaaagaagagaagaaggaatttagtgctcacagtatacccgcttttttgggttggactaaagaagacctttgcagtattaatggtaagtcactgggcaacattctacattttaattgtttcttgagattagag

SEQ ID NO: 31 - DNA, 236 bp, Mus musculus
ttagacggggtttaaaatggactttccttttcgctctgattctcctttaggagcctttggacatcttgccattttcaattcttgtctgcaaaccagaagtatagacgacaaacagctattgcatggcatattaaaaattataacaagctggaggaaacagcatgaagatatttttctttttagctggtaggtagtactttattcccacattaaaatgtgggcccagattctgtatt

SEQ ID NO: 32 - DNA, 251 bp, Mus musculus
tggtttaaaatggaatattctctaattgaatttctttgcctttatcacagtaatctgtcggaagcgagtccagaggtccttggtttaaacatagagatcatgcggttcctttccttgtttctcaagcactgcgcgtacccgctcccgctggcagacagtgaatgggacttcatcatgtgctccatgctggcttggttggaggtaaatcagcccgatgtgtcatcccctgagttgggcctttgcacactctg

SEQ ID NO: 33 - DNA, 314 bp, Mus musculus
ggtgttgggtagcaccttgcttacctgttttatttctgtcctttccttagacaaccagtgagaaccaggctctgtactctgttccacttgtgcagctgtttgcctgtgtcagctttgatctggcctgtgatctctgtgccttttttgactcaataactccagatattgttgacaatcttcctgtaaatctcatcagtgagtggaaagagtttttttctaaaggcatccacagtttgctattacctcttttggtaaatgctatcggtaagtgcaaaggggtatgatgatggtaattattgaaaatggctgttgac

SEQ ID NO: 34 - DNA, 338 bp, Mus musculus
atgtgtgttcttatgagttgtgtgtgtgtgtttcttcatttcaataccaggagaaaacaaagacctatctgaaacgtcctttcagaacgcaatgctgaaacccatgtgtgaaacactaacatacatctccaaggaccagctactgagccacaagctccctgcgagattggttgccagccagaaaacaaacttgccagagcacctccagactctgctgaacactttgaccccactgcttctcttcagagccagacctgtgcaaattgctgcttatcatatgctgtgcaagtaagacattggcagtggcataagtatttggtctctaaagcatgatgcat

SEQ ID NO: 35 - DNA, 175 bp, Mus musculus
tagtagacagttggctataaatgtgttaaaagtgatgtttcgcttttcagactgatgcctgaattgccacagcatgatcaggacaatctgaggtcgtatggagatgaagaggaagaaccagccttgtaagggttttgagtgtgtgtttgcttgtgtgagtgcaggttacaggacc

SEQ ID NO: 36 - DNA, 287 bp, Mus musculus
agatgtttgttcgagacaggtgaatggtcctgtgtgacgttgtttcccaggtcgccgccggccgcgctgatgtctctcctcagctctcaggaggagctgctggagaatgtcctgggctgtgtccctgtgggccagatcgtgaccgttaagccactgagcgaggacttctgctatgtcctgggatacctcctcacttggaagttaatactgactttcttcaaagctgcatcgtctcaggtaaataagttctgggctgttggagggtggggtgctgttgcattgcagtg

SEQ ID NO: 37 - DNA, 272 bp, Mus musculus
tttgtcttggcacagttgagtttactaacacattctttgcttttcaccagcttcgtgctctgtattcaatgtaccttcggaaaacaaagagtctgaataaattactctatcatctcttcagacttatgccagaaaaccctacgtacggagagacagctattgaggtatcaagtaaagaccccaagaccttcttcaccgaggaggttcagctgagtattagaggtcagtgggctacacgtgtgtgggctgcacatgtgtgggctacacgtctg

SEQ ID NO: 38 - DNA, 321 bp, Mus musculus
tctaggtgagtgcacatgtatcactaacctgggatttgtttcttcctcagaaacagcaactcttccgtatcatatcccacacctggcgtgctcggtctatcacatgactttaaaagacttgcctgccatggttaggctatggtggaatagcagtgagaagcgtgtcttcaatattgtagatagatttacaagcaagtatgtcagcaatgttctttcttttcaagaaatatcttctgtacaaacaagtacacagctattcaatggcatgacggttagtattgtcttggtttttttctagagaaatgaataccagttatattt

SEQ ID NO: 39 - DNA, 295 bp, Mus musculus
aaggaaaaataccttttttttgtattgtattgtattgcattgtattgtaggttaaggcgcgagctactactcgagaagtgatggctacgtacaccatcgaagacatagtcattgaactcataatacagttgccttccaattacccactgggctcaataacagtggaaagtgggaagaggatcggggtggctgtgcagcagtggcgaaactggatgctgcagctgagcacgtacctcactcaccaggtatgctgccacgagcgcactggtcactcacactagagcacatctgtctg

SEQ ID NO: 40 - DNA, 268 bp, Mus musculus
catttggagacctttagtgtgtggtgactttctttcctctctactaacagaacggaagtatcatggaaggcttagcattatggaagaataatgtagacaaacggtttgaaggtgttgaagattgtatgatctgcttctcagttattcatggtttcaactattctcttcccaaaaaagcctgtagaacatgcaagaaaaagtttcactcagcttgcctggtaaggcgaagggaaatctcttaacattctttgtggtctgtatatgttat

SEQ ID NO: 41 - DNA, 271 bp, Mus musculus
acgaaagccttacaaatttactttaacattaaaacctttttttttttcagtacaaatggtttacatctagcaacaagtccacttgcccgctctgccgtgagacctttttctgaggtttttttcattggaagttgtcgctgccgtaggtcaagccaaagggaatggattggctccaccttgaagtactgatgtgaagccagtgagcatgacaaagtgccatctgtcagtatgatcctcaaatcaggcaacctctgcagattgctttgtaaat

SEQ ID NO: 42 - DNA, 42 bp, Mus musculus
cttcttcaggtcattgaaaaggtattttagccccctctcccc

SEQ ID NO: 43 - DNA, 42 bp, Mus musculus
cttcttcaggtcattgaaaaggaattttagccccctctcccc

SEQ ID NO: 44 - DNA, 286 bp, Homo sapiens
ccggtggcggatggtcagggacacagcgggaggcgggagcggaagtggggcagcaaatggacagggtgggtggcggaaaagggcccgggggaagttattacagggtgtcctcttccgccgccagaagccggaagttgtgtcccggacgtgtcaaccggggtctgagtgctcagagtacagctgcaaccgcgaccatgggcgggaagaacaagcagcgaactaaagggaacctgagggtgagcgggggctggctcggccgacccgggagcctcgcttcgaggcggct

SEQ ID NO: 45 - DNA, 304 bp, Homo sapiens
tttgaacccttcctggactctgacttttttctccgtaattttcttatcagccttcaaacagtggccgagctgcagaactccttgccaaagaacagggaacagtgcctggatttattggttttggaacatctcagagtgacctaggctatgttcctgctattcaaggagctgaagaaattgacagtcttgtagattctgatttccgaatggtgctgcggaaactttcaaagaaagatgtcaccacaaaattaaaagcaagttttcttgttttcataaaaattatcaagaaaatccctttgttaaa

SEQ ID NO: 46 - DNA, 199 bp, Homo sapiens
gccagttattatggttgttaatcctaatgatgttaataacttacttttaggctatgcaggaatttggaaccatgtgtacagagagagacacagaaactgtgaaaggagttcttccatattggccaagaattttttgcaaaatttcacttgtaagtattaaaactttgctagtttatttctgttgtatattttttggttg

SEQ ID NO: 47 - DNA, 331 bp, Homo sapiens
ttgtttttatgggctagagctattttaaatcacatttatatttttaataggatcatgaccgtcgcgtccgagaagccacacaacaagcttttgaaaaacttatccttaaagtaaagaaacagttggctccctacttaaaaagtttaatgggatattggctaatggctcagtgtgatacttacacaccagctgcgtttgcagcaaaagatgcatttgaagcggcttttcctccaagcaagcaacctgaagccatagcattttgtaaggatgaaattacaagtgtaagttctggaatcattctgaatctattttttttttttaagtatttaag

SEQ ID NO: 48 - DNA, 153 bp, Homo sapiens
cattgacagttcagtaaaaccaaaggctttggtatttcccccctttgtaggtgctgcaggatcatcttataaaagaaacacctgatacactcagtgacccgcagtaagttgtattgtttcattgtaactcatgttaaggatttgtttcactca

SEQ ID NO: 49 - DNA, 281 bp, Homo sapiens
gtttggctaattctaaatttttaaatatatttattttgttttatatcaagaactgttccagaggaagaaagagaagctaaattctaccgggttgtaacttgttccttattggcattaaagagattactttgccttttacctgataatgagcttgattctctggaggagaaatttaagtctcttttatcacagaataagttttggaagtatggaaaacacagtgtacctcaggtatattaatcttttttatcttaagacatttctctgattccttaccccca

SEQ ID NO: 50 - DNA, 274 bp, Homo sapiens
ttatcaagttttactaggttttaatttaattcagctttgcattcttttagattcgctcagcttattttgagttagtctctgcattgtgccagcgcattccacagttgatgaaagaggaagcatccaaagtgagcccatcagttctacttagcattgatgacagtgacccaattgtctgcccagctctctgggaagctgtactctatacacttacaactattgaggtatgtaagagaggcacatttagtacactgaggaatgaacctatgagata

SEQ ID NO: 51 - DNA, 291 bp, Homo sapiens
aaagtatgtttatgatctctaattactactataatcttttgtttggttaggactgttggcttcatgtaaatgcaaaaaagagtgtgtttcccaagctatcaactgtgattcgtgaaggtggtcggggtctagctactgtcatatatccttaccttctgccattcatcagcaagctccctcagtccatcacaaatccaaagttggatttcttcaaaaatttcctcacgtctctagttgctgggtaagtaatttaaatttttgatttttaaaacaaaacagattttcttgttt

SEQ ID NO: 52 - DNA, 236 bp, Homo sapiens
tcttgtttcttcttcatgaaagagtaatccagttattaattatttcttaggctgtcaacagagagaactaaaaccagctctttagagtcctcggcagtaatatctgctttttttgaatgcttacgttttataatgcagcaaaacttaggtgaggaagagattgaacagatgctcgtcaatgatcaggtatctataatgtaaaagtcgtcagtctctttgcatactgattatgtagg

SEQ ID NO: 53 - DNA, 910 bp, Homo sapiens
ggggttgaagaccaaatatatattttatgatattctaccttttctttcagttgatcccttttattgatgcagttctcaaagacccaggattgcaacatgggcagctatttaaccatttagcagaaactctaagttcctgggaagccaaagcagacacggaaaaagatgaaaaaacagctcacaacttggagaacgtactgatacatttctgggaaagactgtcagagatctgtgttgcgaaaatcagtgagccagaagctgatgttgagtccgttttgggtgtatctaacctattacaggtgcttcagaagccgaagagctcattgaagtcaagtaaaaaaaaaaatggtaaggttagatttgctgatgagatacttgaaagcaataaagagaatgaaaaatgtgtatcttcagaaggagagaagattgaaggctgggaattaacaactgaaccttctctcactcataattcttcaggccttttgtctcctctaaggaaaaaacctttggaagacttagtctgtaaactcgcagatataagtattaattatgtcaatgaacgaaagtcagagcaacatctaaggtttctttctactctgcttgactccttttcttcaagccgagtatttaaaatgctacttggtgatgaaaaacagagtattgtccaagccaaacctcttgaaatagccaagcttgtacaaaaaaatcctgcggtgcagtttttataccagaaactgataggttggctaaatgaagatcaaaggaaggattttggtttcctggtggacattttgtacagtgctctccggtgctgtgacaatgatatggaaagaaaaaaagtcttggatgatctaaccaaggtattcctgttgtatatcttttcaaactatttgaataatagatgagtaga

SEQ ID NO: 54 - DNA, 142 bp, Homo sapiens
aacaaaaagttgttctagcagattgtttctaactctttttcttttttcaggtggacttgaaatggaattctcttcttaagattattgaaaaggtatcttagggatttttttttctttttttttttgtatttatgggaataga

SEQ ID NO: 55 - DNA, 290 bp, Homo sapiens
gagagtatttgatcttgctgttaatctttgctttaattttatggttttaggcatgtcctagttcagataaacatgctttagtaactccttggctcaaaggcgatatccttggtgagaaattggtcaacttggcagattgtctttgtaatgaggacttggaatccagggtatcttcagaatctcacttctcagaaagatggactcttctaagcttggtattatcccaacatgttaaaaatggtaggacaaatatggcttttgttttctaatggggaatagtccatttctcc

SEQ ID NO: 56 - DNA, 340 bp, Homo sapiens
tacacgttaatatttttattttttgtctgtttgaacctttttttctccagattacttgattggagacgtatatgttgaaagaatcattgttagacttcatgaaactttattcaaaacaaagaaattatcagaagctgaaagcagtgactcatcagtgtcttttatctgtgatgtggcctataactatttcagctcagcgaaaggatgcttgctaatgccatcatctgaagatttattattaactctctttcagttatgtgctcagagcaaagaaaaaacacatttgccaggtaatagcctactgctcaaatgttttgttgggaatctccggctctgacct

SEQ ID NO: 57 - DNA, 254 bp, Homo sapiens
gagagttaattcagtgagattacaaattcctgttgacatcttgattttagattttcttatctgtaaactgaaaaatacttggctctctggtgtaaatttattggttcatcaaactgacagttcatataaagagagtaccttcctacatttgtctgctctgtggctgaagaaccaagttcaggcttcatctttggatatcaacaggtaaccttttttgagcttggtttcagtgtctcttttatacttgaacagtg

SEQ ID NO: 58 - DNA, 245 bp, Homo sapiens
aactagtcatagcgtaactacaacagttaagttctctgcattatttttagtctccaagtcctcttgtctgctgttgatgatttgctaaatacacttctagagagtgaagattcttatcttatgggagtttatattggaagtgtaatgccgaacgacagtgaatgggaaaagatgaggcagtctcttcctatgcaggtattttggaaattgaagagtacatatctcattctgaagtttggatttca

SEQ ID NO: 59 - DNA, 287 bp, Homo sapiens
caattgcttaggtaataaataattttgtatataacgtattcttttcacagtggttacatagacctcttttagagggaagattgagtttgaattatgaatgtttcaaaacagattttaaggaacaggacataaagacacttcccagccatttgtgtacttcagcattattgagcaaaatggtcttaattgcactgagaaaggaaacagtcttagaaaataatgagcttgagaaaataagtaagtatatatgagtatttacatataacataatgcatgaatgaatataa

SEQ ID NO: 60 - DNA, 260 bp, Homo sapiens
ggtttgttcacagagtgcttttcataatttggtgctgtctcttcttttagttgcagaactgctttattcactgcagtggtgtgaagaattagataacccacctatttttctaattggattttgtgaaatacttcaaaaaatgaatattacgtatgataacttacgtgtacttggtaatacgtcgggccttttgcagctgttatttaacaggtaagaatctctttcaatttgtttttaaaatgactatgctacttctttat

SEQ ID NO: 61 - DNA, 205 bp, Homo sapiens
attcagtatgctttcaaaacggaatataatttttttctttcctttttcaggtccagagaacatggcacactgtggtctcttattattgctaagttgatcctttcccgaagcatttcatctgatgaagtaaaaccacattataagagaaaagaaaggtattcttatttaaaatgtttttactttgtagtttactgtaatcggtcac

SEQ ID NO: 62 - DNA, 243 bp, Homo sapiens
actgtatgtttggaagaaatctaacttgtgttttttttttctttaaacagtttttttccactaactgaaggcaatttgcataccattcaaagtctatgtccatttttgtcaaaagaagaaaagaaagaatttagtgctcaatgtatacctgctcttttgggctggactaagaaagatctttgcagcactaatggttagttgcttgataataaatatttcattaatttaattattgcttgagat

SEQ ID NO: 63 - DNA, 236 bp, Homo sapiens
gcgatatagttcttaaatatttttaattttcattttgaattttactttaggaggttttggacatcttgccattttcaattcttgtctgcaaaccaaaagtatagatgatggagagctattacatggaatattaaaaatcataatatcctggaagaaagagcatgaagatatttttcttttcagttggtaggtgatactttatactatgtttttccttcagtatacaattcagacaa

SEQ ID NO: 64 - DNA, 245 bp, Homo sapiens
tgaatgtcaatataatcttttattgtcttttgtttttatctttatcacagtaatctatcagaagcaagtccagaggtactgggtgtaaatatagaaataatccggtttctttccctatttctgaaatactgctcatcccctttggcagagagtgagtgggacttcatcatgtgctccatgttggcttggttggaggtaaattaacccgatatgtcatcacctcttgtgggtttttgtacatactt

SEQ ID NO: 65 - DNA, 314 bp, Homo sapiens
gtgttctggtagaagtctgtttacctatttcatttttattgttttttcagacaacaagtgagaatcaggcattgtattctattccacttgtgcaactgtttgcctgtgtcagctgtgatttggcctgtgacctcagtgctttctttgattccacaactctggataccattggcaatcttcctgtaaatctaatcagtgaatggaaagaatttttttcccaaggcatccacagtttgcttttacctattttggtgactgttacaggcaagtgaaaaagggaataatagtgagattgattcattggaaatgactta

SEQ ID NO: 66 - DNA, 338 bp, Homo sapiens
atactaactttcttttgagacgtgcatgtttctttcctttttgtcattaggagaaaacaaagatgtgtctgaaacatcctttcagaatgcaatgctgaaacccatgtgtgaaacattaacgtatatctcaaaggaacagctattgagtcacaaacttcctgcaagattagttgctgaccaaaaaacaaacttaccagaatatctccagactttgttaaatacattggccccattactcctcttcagagctaggcctgtgcaaattgctgtttatcatatgctatacaagtaagaattcatccaattgaatcaatgttacagtggtctaaaaaaataga

SEQ ID NO: 67 - DNA, 175 bp, Homo sapiens
taataaacagtttaatatcacatatttaaaaataatgttctaatttctagattgatgcctgaattaccacagtatgatcaggataatctaaagtcatacggagatgaagaagaagagccagccttgtaaggtttttttaaataatttgttttattaaattcttataatccatctc

SEQ ID NO: 68 - DNA, 287 bp, Homo sapiens
aaggaatgaattataaagaaaacttgtggtgtttaatcattgattcttaggtcaccaccagcagcactgatgtctcttcttagcattcaagaggacttactagaaaatgttttggggtgtattcctgttggacagatagttactattaaaccactgagtgaagacttctgttatgttctgggataccttctcacttggaaattaatactaactttcttcaaagctgcatcatcacaggtaaataaatatgtgacaactttcgatagttctgtcctaatatgcttctg

SEQ ID NO: 69 - DNA, 272 bp, Homo sapiens
aattgtcttggtgtatttggtttactaacaaattctttgctctccaccagcttcgggctttgtattccatgtatcttcggaaaacaaagagtttgaataaattgctctatcacctgttcaggcttatgccagaaaatccaacctatgcagagacagcagttgaggtcccaaataaggaccctaaaacattctttactgaggagctccagctgagtattagaggtcagtaagatatgtgtttatgttctttcttggacactagattcagacta

SEQ ID NO: 70 - DNA, 321 bp, Homo sapiens
ttctatataagaaatatatgttactaatcaataatttgttttttcctcagaaacaacaatgcttccataccacattccacacttggcttgttcagtctatcatatgacattaaaagacttgcctgccatggttaggttgtggtggaatagcagtgagaagcgtgttttcaatattgtggatagatttacaagcaagtatgtcagcagtgttctttcttttcaagaaatatcttctgtacaaacaagtacacaactatttaatggcatgacggttagtattgtcttgactttctctagaaaagttgttttaatattgggtat

SEQ ID NO: 71 - DNA, 295 bp, Homo sapiens
ggttgaaagacagaaatttggcaaacaaaatatcctttgttgtattgcaggttaaagctcgagctactactcgagaggtaatggctacttatactattgaggacatagttattgaacttataatacaactgccttcaaattatccactgggttcaataatagtagaaagtgggaaaagagtaggagtagctgttcagcagtggcggaactggatgctgcagttaagcacttacctcacccatcaggtaagtttctgtttacacatttggctttacaaacttggaaaagatgatct

SEQ ID NO: 72 - DNA, 268 bp, Homo sapiens
aaattggtggttatcagtgtgtagtgacttatttttcttcttgttaacagaatggaagtattatggaaggcttagctttatggaaaaataacgtagacaaacgttttgagggtgttgaagattgcatgatctgtttctcagtcattcacggtttcaactattcccttcccaaaaaagcctgtagaacatgcaagaaaaaattccattcagcctgcttggtaagtctaaagagaaattaacttacttatattttatgtattttatacac

SEQ ID NO: 73 - DNA, 163 bp, Homo sapiens
gaggagaactttaaaagttcttttagtatttaaaaccttttctttttcagtacaaatggtttacatctagcaacaaatccacttgtccactgtgtcgtgagacgtttttctgagatttttttcactggaagggatccctgaagtacatcaaacaaaggcattg

SEQ ID NO: 74 - DNA, 44 bp, Homo sapiens
cttcttaagattattgaaaaggtatcttagggatttttttttct

SEQ ID NO: 75 - DNA, 44 bp, Homo sapiens
cttcttaagattattgaaaaggaatcttagggatttttttttct

SEQ ID NO: 76 - DNA, 22 bp, Mus musculus
cattgaaaaggtattttagccc

SEQ ID NO: 77 - DNA, 22 bp, Mus musculus
cattgaaaaggtattttagccc

SEQ ID NO: 78 - DNA, 22 bp, Mus musculus
cattgaaaaggaattttagccc

SEQ ID NO: 79 - DNA, 22 bp, Mus musculus
cattgaaaaggaattttagccc

SEQ ID NO: 80 - protein, 4 aa, Artificial Sequence
  Description of Artificial Sequence Synthetic zinc finger motif
CCHH

SEQ ID NO: 81 - protein, 4 aa, Artificial Sequence
  Description of Artificial Sequence Synthetic zinc finger motif
CCCC

SEQ ID NO: 82 - protein, 4 aa, Artificial Sequence
  Description of Artificial Sequence Synthetic zinc finger motif
CCHC

SEQ ID NO: 83 - protein, 8 aa, Artificial Sequence
  Description of Artificial Sequence Synthetic zinc finger motif
CCCHCCCC

SEQ ID NO: 84 - protein, 8 aa, Artificial Sequence
  Description of Artificial Sequence Synthetic zinc finger motif
CCCHHCCC